(19) United States
(12) Patent Application Publication
(10) Pub. No.: US 2023/0120825 A1
(43) Pub. Date: Apr. 20, 2023

(54) Compositions, Methods, and Systems for Paternity Determination

(71) Applicant: Laboratory Corporation of America Holdings, Burlington, NC (US)

(72) Inventors: Jonathan Williams, San Diego, CA (US); John A. Tynan, San Diego, CA (US); Eric O'Neill, San Diego, CA (US); Roy Brian Lefkowitz, San Diego, CA (US)

(21) Appl. No.: 17/802,934

(22) PCT Filed: Feb. 26, 2021
(86) PCT No.: PCT/US2021/020021

§ 371 Date: Aug. 26, 2022

Related U.S. Application Data
(60) Provisional application No. 62/983,491, filed on Feb. 28, 2020.

(51) Int. Cl. C12Q 1/6858; C12Q 1/6888

(52) U.S. Cl. CPC C12Q 1/6858; C12Q 1/6888; C12Q 2600/156; C12Q 2600/172

(57) ABSTRACT

This application provides methods and systems for paternity determination. In some embodiments, the method is a non-invasive prenatal paternity determination method, which comprises obtaining genotypes for one or more polymorphic nucleic acid targets in a genomic DNA sample obtained from an alleged father, isolating cell-free nucleic acids from a biological sample obtained from the pregnant mother comprising fetal nucleic acids. The amount of each allele of one or more polymorphic nucleic acid targets in cell-free nucleic acids are determined and informative polymorphic nucleic acid targets are identified. Next, the allele frequency of each allele of the selected informative polymorphic nucleic acid targets is measured and fetal genotypes for each selected informative polymorphic nucleic acid targets are determined based on the allele frequency. Finally, the paternity status of the fetus are determined based on the genotypes of the mother, alleged father and the fetus for the informative nucleic acid targets.

### FIELD

The technology in part relates to methods and systems used for determining paternity.

### BACKGROUND

Paternity determination is to determine whether an individual is the biological father of another individual. In some cases, it is desirable to determine paternity at the prenatal stage, i.e., before birth. While prenatal paternity tests, involving chorionic villus sampling or amniocentesis, are highly accurate, they require invasive procedures such as retrieving placental tissue or inserting a needle through the mother’s abdominal wall. Non-invasive prenatal paternity rests have recently been developed; however, because the amount of fetal DNA in cell-free samples from the pregnant mother is very low, and the cell-free DNA is highly fragmented samples, the accuracy of the current non-invasive paternity tests remains a concern.

### SUMMARY OF THE INVENTION

The present invention provides non-invasive methods of prenatal paternity determination using a panel of polymorphic nucleic acid targets. The panel can be amplified in a multiplexed fashion and analyzed by sequencing. The method quantifies the presence of fetus-specific alleles in samples having a mixed maternal and fetal DNA and determines the genotype of the fetus. The genotypes of the trio (i.e., the mother, the fetus, and the alleged father) are then analyzed to produce a paternity index, which represents the likelihood that the alleged father is the biological father versus the likelihood that a random man, from the same population as the alleged father, is the biological father. This method is fast, convenient and accurate in determining paternity.

In some embodiments, disclosed herein is a method of determining paternty of a fetus in a pregnant mother. The method comprises (a) obtaining genotypes for one or more polymorphic nucleic acid targets in a genomic DNA sample obtained from an alleged father, (b) isolating cell-free nucleic acids from a biological sample obtained from the pregnant mother comprising fetal nucleic acids; (c) measuring the frequency of each allele of one or more polymorphic nucleic acid targets in cell-free nucleic acids;(d) select informative polymorphic nucleic acid targets from the one or more polymorphic nucleic acid targets, (e) determining the measured allele frequency of each allele of the selected informative polymorphic nucleic acid targets and thereby determining fetal genotypes based on the measured allele frequency for each selected informative polymorphic nucleic acid targets, and (f) determining paternity status of the fetus based on the genotypes of the mother, alleged father and the fetus for the informative nucleic acid targets. In some embodiments, step (a) further comprises obtaining genotypes for the one or more polymorphic nucleic acid targets in a genomic DNA sample obtained from the pregnant mother. step (e) further comprises by comparing the measured allele frequency to a threshold of respective polymorphic nucleic acid targets. In some embodiments, step (f) further comprises determining paternity index for each informative polymorphic nucleic acid targets, determining a combined paternity index for all informative polymorphic nucleic acid targets, which is the product of the paternity indexes for each informative polymorphic nucleic acid targets. In some embodiments, step (c) comprises determining measured allele frequency based on the amount of each allele of one or more polymorphic nucleic acid targets in cell-free nucleic acids.

In some embodiments, the informative polymorphic nucleic acid targets are selected by performing a computer algorithm on a data set consisting of measurements of the one or more polymorphic nucleic acid targets to form a first cluster and a second cluster, wherein the first cluster comprises polymorphic nucleic acid targets that are present in the mother and the fetus in a genotype combination of AAₘₒₜₕₑᵣ/AB_{fetus}, or BBₘₒₜₕₑᵣ/AB_{fetus}, and/or wherein the second cluster comprises SNPs that are present in the mother and the fetus in a genotype combination of ABₘₒₜₕₑᵣ/BB_{fetus} or ABₘₒₜₕₑᵣ/AA_{fetus}.

In some embodiments, the paternity index is determined by inputting the genotypes of the mother and alleged father and fetal genotypes for each of the informative polymorphic nucleic acid targets into a paternity determination software. In some embodiments, the alleged father is determined to be a biological father if the combined paternity index is greater than a predetermined threshold.

Also provided is a system for determining paternity comprising one or more processors; and memory coupled to one or more processors, the memory encoded with a set of instructions configured to perform a process comprising: obtaining genotypes for one or more polymorphic nucleic acid targets in a genomic DNA sample obtained from an alleged father, determining the amount of each allele of one or more polymorphic nucleic acid targets in cell-free nucleic acids from a sample obtained from a pregnant mother, select informative polymorphic nucleic acid targets from the one or more polymorphic nucleic acid targets, determining the measured allele frequency of each allele of the selected informative polymorphic nucleic acid targets and thereby determining fetal genotypes based on the allele frequency for each selected informative polymorphic nucleic acid targets, and determining the paternity status of the fetus based on the genotypes of the mother, alleged father and the fetus for the informative nucleic acid targets.

Also provided is a non-transitory machine readable storage medium comprising program instructions that when executed by one or more processors cause the one or more processors to perform any one of the methods of determining paternity status described above.

### BRIEF DESCRIPTION OF THE DRAWINGS

The drawings illustrate exemplary embodiments of the technology herein and are not limiting. For clarity and ease of illustration, the drawings are not made to scale and, in some instances, various aspects may be shown exaggerated or enlarged to facilitate an understanding of particular embodiments.

FIG. 1 shows an exemplary workflow the paternity determination method described herein.

FIG. 2 shows an illustrative embodiment of a system in which certain embodiments of the technology may be implemented.

FIG. 3 shows expected versus detected fetal fractions in a synthetic mixture modeling maternal DNA and fetal DNA. X-axis represents the SNV determined mixture ratio based on the sequencing measured reference allele frequency. Y-axis represents the expected mixture fraction based on fluorescent quantitation of DNAs used to prepare the mixtures.

FIG. 4 shows the number of identified child heterozygous/materal homozygous loci as compared to the potential number of child heterozygous/materal homozygous loci as determined by child genomic DNA genotyping.

FIG. 5 shows likelihood ratios of paternity (paternity index) based on informative SNVs for which the mother is homozygous and the child is heterozygous in samples containing mixtures of maternal and child DNA. “Included father’ means that the test confirmed that the alleged father is the biological father of the child. “excluded father” means that the test result was 0, which indicates that the alleged father is not the biological father

FIG. 6 shows replicate determination of fetal fraction based on informative SNVs for which the child is heterozygous and the mother is homozygous. The maternal genomic DNA was not available for genotyping. Two replicates (identified by RDSR numbers) from each cf DNA sample (identified by SQcfDNA numbers) were tested.

FIG. 7 shows replicate determination of the number of informative SNVs for which the child is heterozygous for the cfDNA samples analyzed in the same experiment as shown in FIG. 6. The maternal genomic DNA was not available for genotyping. Two replicates (identified by RDSR numbers) from each cfDNA sample (identified by SQcfDNA numbers) were tested.

FIG. 8 shows Median and MAD for homozygous allele frequencies of SNPs having different reference allele and alternate allele combination (“Ref Alt combination”). A higher median and a higher MAD for SNPs having A_G, G_A, C_T, or T_C combinatons were observed.

FIG. 9 shows that distribution of Ref Alt combinations. A G, G_A, C_T, and T_C are the most frequent combinations of reference and alternate allele in a v1.1 panel (i.e. a combination of subsets of Panel A and Panel B as disclosed in Table 1), occurring in 79.5% of the panel’s targets (172 out of the 219 donor fraction assays).

FIGS. 10A and 10B illustrate an embodiment in which an allele-specific probe pair consisting of probes ① and ② are designed to detect an allele A (reference allele) at an SNV locus. probes ① and ② are immediately adjacent to each other when hybridized to the target nucleic acid molecule, i.e., there is no nucleotide between the two probes’ proximal ends. In this embodiment, probe ① is hybridized to a sequence that is 5′ to the sequence to which probe ② hybridizes. Probe ② contains a T at its 5′ end, which hybridizes to the A at the SNV locus (FIG. 10A) and will not hybridize to a G (an alternate allele at the same locus) (FIG. 10B). In this specific embodiment, the nucleotide complementary to the detected allele is at the 3′ end of one probe. In other embodiments, the nucleotide complementary to the detected allele A can also be at the 5′ end of probe ①.

### DEFINITIONS

The terms “nucleic acid” and “nucleic acid molecule” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), transfer RNA (tRNA), microRNA), DNA or RNA analogs (e.g., containing base analogs, sugar analogs and/or a non-native backbone and the like), and/or RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. Nucleic acids can be in any form useful for conducting processes herein (e.g., linear, circular, supercoiled, single-stranded, double-stranded and the like) or may include variations (e.g., insertions, deletions or substitutions) that do not alter their utility as part of the present technology. A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), single nucleotide variants (SNVs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil. A template nucleic acid may be prepared using a nucleic acid obtained from a subject as a template.

The term “polymorphism” or “polymorphic nucleic acid target” as used herein refers to a sequence variation between different alleles of the same genomic sequence. A sequence that contains a polymorphism is considered a “polymorphic sequence”. Detection of one or more polymorphisms allows differentiation of different alleles of a single genomic sequence or between two or more individuals. As used herein, the term “polymorphic marker”, “polymorphic sequence”, “polymorphic nucleic acid target” refers to segments of genomic DNA that exhibit heritable variation in a DNA sequence between individuals. Such markers include, but are not limited to, single nucleotide variants (SNVs), restriction fragment length polymorphisms (RFLPs), short tandem repeats, such as di-, tri- or tetra-nucleotide repeats (STRs), variable number of tandem repeats (VNTRs), copy number variants, insertions, deletions, duplications, and the like. Polymorphic markers according to the present technology can be used to specifically differentiate between a maternal and fetal allele in the enriched fetus-specific nucleic acid sample and may include one or more of the markers described above.

The terms “single nucleotide variant” or “SNV” (used interchangeably with “single nucleotide polymorphism” or “SNP”) as used herein refer to the polynucleotide sequence variation present at a single nucleotide residue between different alleles of the same genomic sequence. This variation may occur within the coding region or non-coding region (i.e., in the promoter or intronic region) of a genomic sequence, if the genomic sequence is transcribed during protein production. Detection of one or more SNVs allows differentiation of different alleles of a single genomic sequence or between two or more individuals.

The term “allele” as used herein is one of several alternate forms of a gene or non-coding regions of DNA that occupy the same position on a chromosome. The term allele can be used to describe DNA from any organism including but not limited to bacteria, viruses, fungi, protozoa, molds, yeasts, plants, humans, non-humans, animals, and archeabacteria. A polymorphic nucleic acid target disclosed herein may have two, three, four, or more alternate forms of a gene or non-coding regions of DNA that occupy the same position on a chromosome. A polymorphic nucleic acid target that has two alternate forms is commonly referred to as a bialleilic polymorphic nucleic acid target. For the purpose of this disclosure, one allele is referred to as the reference allele, and the others are referred to as alternate alleles. In some embodiments, the reference allele is an allele present in one or more of the reference genomes, as released by the Genome Reference Consortium (www.ncbi.nlm.nih.gov/grc). In some embodiments, the reference allele is an allele present in reference genome GRCh38. See, www.ncbi.nlm.nih.gov/grc/human. In some embodiments, the reference allele is not an allele present in the one or more of the reference genomes, for example, the reference allele is an alternate allele of an allele found in the one or more of the reference genomes.

The terms “ratio of the alleles” or “allelic ratio” as used herein refer to the ratio of the amount of one allele versus the amount of the other allele in a sample.

The term “Ref_Alt” combination with regard to an SNV refers to a combination of the reference allele and alternate allele for the SNV in the population. For example, a Ref_Alt of C_G refers to that the reference allele is C and the alternate allele is G for the SNV.

The terms “amount” or “copy number” as used herein refers to the amount or quantity of an analyte (e.g., total nucleic acid or fetus-specific nucleic acid). The present technology provides compositions and processes for determining the absolute amount of fetus-specific nucleic acid in a mixed recipient sample. The amount or copy number represents the number of molecules available for detection, and may be expressed as the genomic equivalents per unit.

The term “fraction” refers to the proportion of a substance in a mixture or solution (e.g., the proportion of fetus-specific nucleic acid in a recipient sample that comprises a mixture of recipient and fetus-specific nucleic acid). The fraction may be expressed as a percentage, which is used to express how large/small one quantity is, relative to another quantity as a fraction of 100.

The term “sample” as used herein refers to a specimen containing nucleic acid. Examples of samples include, but are not limited to, tissue, bodily fluid (for example, blood, serum, plasma, saliva, urine, tears, peritoneal fluid, ascitic fluid, vaginal secretion, breast fluid, breast milk, lymph fluid, sputum, cerebrospinal fluid or mucosa secretion), or other body exudate, fecal matter (e.g., stool), an individual cell or extract of the such sources that contain the nucleic acid of the same, and subcellular structures such as mitochondria, using protocols well established within the art.

The term “blood” as used herein refers to a blood sample or preparation from a subject. The term encompasses whole blood or any fractions of blood, such as serum and plasma as conventionally defined.

The term “target nucleic acid” as used herein refers to a nucleic acid examined using the methods disclosed herein to determine if the nucleic acid is fetal or maternal-derived cell free nucleic acid.

The term “sequence-specific” or “locus-specific method” as used herein refers to a method that interrogates (for example, quantifies) nucleic acid at a specific location (or locus) in the genome based on the sequence composition. Sequence-specific or locus-specific methods allow for the quantification of specific regions or chromosomes.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), where the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Primers” as used herein refer to oligonucleotides that can be used in an amplification method, such as a polymerase chain reaction (PCR), to amplify a nucleotide sequence based on the polynucleotide sequence corresponding to a particular genomic sequence. At least one of the PCR primers for amplification of a polynucleotide sequence is sequence-specific for the sequence.

The term “template” refers to any nucleic acid molecule that can be used for amplification in the technology herein. RNA or DNA that is not naturally double stranded can be made into double stranded DNA so as to be used as template DNA. Any double stranded DNA or preparation containing multiple, different double stranded DNA molecules can be used as template DNA to amplify a locus or loci of interest contained in the template DNA.

The term “amplification reaction” as used herein refers to a process for copying nucleic acid one or more times. In some embodiments, the method of amplification includes but is not limited to polymerase chain reaction, self-sustained sequence reaction, ligase chain reaction, rapid amplification of cDNA ends, polymerase chain reaction and ligase chain reaction, Q-beta phage amplification, strand displacement amplification, or splice overlap extension polymerase chain reaction. In some embodiments, a single molecule of nucleic acid is amplified, for example, by digital PCR.

As used herein, “reads” are short nucleotide sequences produced by any sequencing process described herein or known in the art. Reads can be generated from one end of nucleic acid fragments (“single-end reads”), and sometimes are generated from both ends of nucleic acids (“double-end reads”). In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

The term “cutoff value” or “threshold” as used herein means a numerical value whose value is used to arbitrate between two or more states (e.g. diseased and non-diseased) of classification for a biological sample. For example, if a parameter is greater than the cutoff value, a first classification of the quantitative data is made (e.g. the fetal cell-free nucleic acid is present in the sample derived from the mother); or if the parameter is less than the cutoff value, a different classification of the quantitative data is made (e.g. the fetus-specific cell-free nucleic acid is absent in the sample derived from the mother).

Unless explicitly stated otherwise, the terms “fetus” or “fetal” refers to the unborn offspring of a pregnant “mother” or “maternal” human or animal. For example, the animal can be a mammal, a primate (e.g., a monkey), a livestock animal (e.g., a horse, a cow, a sheep, a pig, or a goat), a companion animal (e.g., a dog, or a cat), a laboratory test animal (e.g., a mouse, a rat, a guinea pig, or a bird), an animal of verterinary significance or economic significance. The term “father” refers to the paternal parent of origin human or animal. As used herein, “alleged father” or “potential father” refers to a male subject who is being tested for paternal relationship to the fetus.

The term “expected allele frequency” refers the allele frequencie observed in a group of individuals having a single diploid genome, e.g., non-pregnant female. In some cases, the expected allele frequency is the median or mean of the allele frequencies in the group of individuals. The expected allele frequency is typically around 0.5 for heterozygous, and around 0 for homozygous for the alternate allele, and around 1 if homozygous for the reference allele. When the fetus and mother are of the same genotype, the allele frequency in the sample from the pregnant mother is equal to the expected allele frequency.

The term “paternity” refers to the identity of the father, or male parent of origin, for a fetus or child. In some embodiments, paternity for a fetus or child is determined among one or more potential fathers.

One or more “prediction algorithms” may be used to determine significance or give meaning to the detection data collected under variable conditions that may be weighed independently of or dependently on each other. The term “variable” as used herein refers to a factor, quantity, or function of an algorithm that has a value or set of values. For example, a variable may be the design of a set of amplified nucleic acid species, the number of sets of amplified nucleic acid species, percent fetal genetic contribution tested, or percent maternal genetic contribution tested. The term “independent” as used herein refers to not being influenced or not being controlled by another. The term “dependent” as used herein refers to being influenced or controlled by another. Such prediction algorithms may be implemented using a computer as disclosed in more detail herein.

One of skill in the art may use any type of method or prediction algorithm to give significance to the data of the present technology within an acceptable sensitivity and/or specificity. For example, prediction algorithms such as Chi-squared test, z-test, t-test, ANOVA (analysis of variance), regression analysis, neural nets, fuzzy logic, Hidden Markov Models, multiple model state estimation, and the like may be used. One or more methods or prediction algorithms may be determined to give significance to the data having different independent and/or dependent variables of the present technology. And one or more methods or prediction algorithms may be determined not to give significance to the data having different independent and/or dependent variables of the present technology. One may design or change parameters of the different variables of methods described herein based on results of one or more prediction algorithms (e.g., number of sets analyzed, types of nucleotide species in each set). For example, applying the Chi-squared test to detection data may suggest that specific ranges of fetus-specific cell free nucleic acids are correlated to a higher likelihood of confirming paternity.

In certain embodiments, several algorithms may be chosen to be tested. These algorithms can be trained with raw data. For each new raw data sample, the trained algorithms will assign a classification to that sample (e.g., predicted paternal identity). Based on the classifications of the new raw data samples, the trained algorithms’ performance may be assessed based on sensitivity and specificity. Finally, an algorithm with the highest sensitivity and/or specificity or combination thereof may be identified.

### DETAILED DESCRIPTION

### Overview

The present technology relates to analyzing fetal DNA found in blood from a pregnant mother as a non-invasive means to determine paternity of the fetus. This disclosure provides methods of detecting the amount of the one or more cell-free nucleic acids deriving from the fetus that are present in maternal samples.

In some embodiments, the fetal genotype is determined based on the amount of fetus-specific nucleic acids in the cell-free nucleic acids isolated from the pregnant mother. The genotypes of the mother, the fetus, and the alleged father are compared and analyzed to determine the likelihood the alleged father is the biological father of the fetus. The fetus specific nucleic acids are quantified based on measurements of fetus-specific allele for one or more informative polymorphic nucleic acid targets. Various approaches can be used to select informative polymorphic nucleic acid targets, as described below. In some embodiments, the polymorphic nucleic acid targets are single nucleotide variants selected from Table 1 or Table 5. The method typically uses a panel of SNVs that are less than 1000 SNVs, which are cost effective and simplify work flow. In addition, the various steps are used to reduce noise. For example the methods only focus on SNVs having low background with high prevalence across populations. In some cases, the methods incorporation of total copy number competitors for inclusion as a QC monitor. In some embodients, the methods use computer algorithm that allows user to infer genotypes of maternal sample when the genomic maternal DNA is not available.

Therefore the methods disclosed herein can be used to conveniently and accurately determine the paternity of a fetus.

### Specific Embodiments

Practicing the technology herein utilizes routine techniques in the field of molecular biology. Basic texts disclosing the general methods of use in the technology herein include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange high performance liquid chromatography (HPLC) as described in Pearson & Reanier, J. Chrom. 255: 137-149 (1983).

### Samples

Provided herein are methods and compositions for analyzing nucleic acid. In some embodiments, nucleic acid fragments in a mixture of nucleic acid fragments are analyzed. A mixture of nucleic acids can comprise two or more nucleic acid fragment species having different nucleotide sequences, different fragment lengths, different origins (e.g., genomic origins, fetal vs. maternal origins, cell or tissue origins, sample origins, subject origins, and the like), or combinations thereof.

Nucleic acid or a nucleic acid mixture utilized in methods and apparatuses described herein often is isolated from a sample obtained from a subject. A subject can be any living or non-living organism, including but not limited to a human, a non-human animal. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female.

Nucleic acid may be isolated from any type of suitable biological specimen or sample. Non-limiting examples of samples include, tissue, bodily fluid (for example, blood, serum, plasma, saliva, urine, tears, peritoneal fluid, ascitic fluid, vaginal secretion, breast fluid, breast milk, lymph fluid, cerebrospinal fluid or mucosa secretion), lymph fluid, cerebrospinal fluid, mucosa secretion, or other body exudate, fecal matter (e.g., stool), an individual cell or extract of the such sources that contain the nucleic acid of the same, and subcellular structures such as mitochondria, using protocols well established within the art. As used herein, the term “blood” encompasses whole blood or any fractions of blood, such as serum and plasma as conventionally defined, for example. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to further preparation. A fluid or tissue sample from which nucleic acid is extracted may be acellular. In some embodiments, a fluid or tissue sample may contain cellular elements or cellular remnants. In some embodiments, fetal cells or cancer cells may be included in the sample.

A sample often is heterogeneous, by which is meant that more than one type of nucleic acid species is present in the sample. For example, a heterogeneous nucleic acid sample can include, but is not limited to, (i) fetus derived and mother derived nucleic acid, (ii) cancer and non-cancer nucleic acid, (iii) pathogen and host nucleic acid, and more generally, (iv) mutated and wild-type nucleic acid. A sample may be heterogeneous because more than one cell type is present, such as a fetal cell and a maternal cell, a cancer and non-cancer cell, or a pathogenic and host cell. In some embodiments, a minority nucleic acid species and a majority nucleic acid species is present.

The methods described herein can be used for paternity determination for postnatal (after birth) or prenatal (before delivery) samples. For prenatal testing, samples can be taken at one or more time points during pregnancy, during the first, second, or third trimester. In some embodiments, the time points are at least one month after conception, e.g., at least two months, at least three months, at least four months, at least five months, at least six months, at least seven months, at least eight months, after conception. In some cases, where the paternity test for one sample taken during the early stage of pregnancy is inconclusive, one or more additional samples can be taken at a later stage of pregnancy.

In some embodiments, the genotype of the mother can be determined from sequencing the polymorphic nucleic acid targets in genomic DNAs from samples, e.g., buccal swab or buffy coats.

### Samples

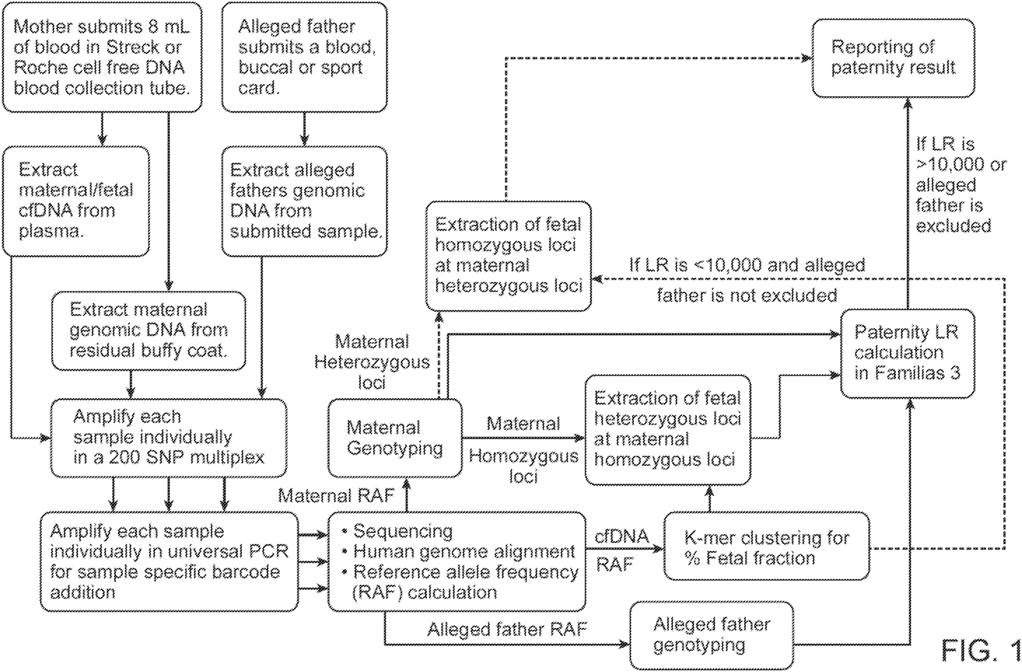

Various samples are used in the paternity determination test disclosed herein. Fetal genotypes are determined using e.g., plasma, blood, serum samples from the pregnant mother. These samples are processed to produce cell-free nucleic acids, as disclosed below, in order to determine fetal genotype. The genotype for the alleged father can be determined from any tissue/cells or body fluids from the alleged father, e.g., buccal swab. The genotype for the mother can also be determined, if needed, using any tissue/cells or body fluids which contains only the maternal DNA (i.e., the sample is free of fetal DNA), for example, the buccal cells or buffy coats. In some cases, the maternal genomic DNA and cell-free DNA are obtained from the same blood sample obtained from the pregnant mother: one fraction of the blood sample is processed to extrace cell-free DNA for fetal genotyping and another fraction is processed for extraction of genomic DNA for maternal genotyping (see FIG. 1).

### Blood Samples

Collection of blood from a subject can be performed in accordance with the standard protocol hospitals or clinics generally follow. An appropriate amount of peripheral blood, e.g., typically between 5-50 ml, is collected and may be stored according to standard procedure prior to further preparation. Blood samples may be collected, stored or transported in a manner known to the person of ordinary skill in the art to minimize degradation or the quality of nucleic acid present in the sample.

### Serum or Plasma Samples

In some embodiments, the sample is a serum sample or a plasma sample. The methods for preparing serum or plasma from recipient blood are well known among those of skill in the art. For example, a pregnant mother’s blood can be placed in a tube containing EDTA or a specialized commercial product such as Vacutainer SST (Becton Dickinson, Franklin Lakes, N.J.) to prevent blood clotting, and plasma can then be obtained from whole blood through centrifugation. On the other hand, serum may be obtained with or without centrifugation-following blood clotting. If centrifugation is used, it is typically, though not exclusively, conducted at an appropriate speed, e.g., 1,500-3,000 times g. Plasma or serum may be subjected to additional centrifugation steps before being transferred to a fresh tube for DNA extraction.

Methods for preparing serum or plasma from blood obtained from a subject (e.g., a pregnant mother or an alleged father) are known. For example, a subject’s blood (e.g., a pregnant mother’s blood) can be placed in a tube containing EDTA or a specialized commercial product such as Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

### Cellular Nucleic Acid Isolation and Processing

Various methods for extracting DNA from a biological sample are known and can be used in the methods of determining paternity. The general methods of DNA preparation (e.g., described by Sambrook and Russell, Molecular Cloning: A Laboratory Manual 3d ed., 2001) can be followed; various commercially available reagents or kits, such as Qiagen’s QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), and GFX™ Genomic Blood DNA Purification Kit (Amersham, Piscataway, N.J.), may also be used to obtain DNA from a blood sample from a subject. Combinations of more than one of these methods may also be used.

In some cases, cellular nucleic acids from samples are isolated. Samples containing cells are typically lysed in order to isolate cellular nucleic acids. Cell lysis procedures and reagents are known in the art and may generally be performed by chemical, physical, or electrolytic lysis methods. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. High salt lysis procedures also are commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, one solution can contain 15 mM Tris, pH 8.0; 10 mM EDTA and 100 ug/ml Rnase A; a second solution can contain 0.2 N NaOH and 1% SDS; and a third solution can contain 3 M KOAc, pH 5.5. These procedures can be found in Current Protocols in Molecular Biology, John Wiley & Sons, N.Y., 6.3.1-6.3.6 (1989), incorporated herein in its entirety.

### Isolating Cell Free DNA From Pregnant Mothers

In some embodiments, the cell-free nucleic acids are isolated from a sample. The term “cell-free DNA”, also referred to as “cell-free circulating nucleic acid” or “extracellular nucleic acid”, refers to nucleic acid isolated from a source having no detectable cells, although the source may contain cellular elements or cellular remnants. As used herein, the term “obtain cell-free circulating sample nucleic acid” includes obtaining a sample directly (e.g., collecting a sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”).

Cell-free nucleic acids isolated from a pregnant mother can include different nucleic acid species, and therefore is referred to herein as “heterogeneous” in certain embodiments. For example, blood serum or plasma from a pregnant mother can include maternal cell-free nucleic acid (also referred to as mother-specific nucleic acid) and fetal cell-free nucleic acid (also referred to as fetus-specific nucleic acid). In some instances, fetal cell-free nucleic acid sometimes is about 1% to about 50% of the overall cell-free nucleic acid (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total cell-free nucleic acid is fetus-specific nucleic acid). In some embodiments, the fraction of fetal cell-free nucleic acid in a test sample is less than about 20%. In some embodiments, the fraction of fetal cell-free nucleic acid in a test sample is less than about 10%. In some embodiments, the fraction of fetal cell-free nucleic acid in a test sample is less than about 5%. In some embodiments, the majority of fetus-specific cell-free nucleic acid in nucleic acid is of a length of about 500 base pairs or less (e.g., about 80, 85, 90, 91, 92, 93, 94, 95, 96, 97, 98, 99 or 100% of fetus-specific nucleic acid is of a length of about 500 base pairs or less). In some embodiments, the majority of fetus-specific nucleic acid in nucleic acid is of a length of about 250 base pairs or less (e.g., about 80, 85, 90, 91, 92, 93, 94, 95, 96, 97, 98, 99 or 100% of fetus-specific nucleic acid is of a length of about 250 base pairs or less). In some embodiments, the majority of fetus-specific cell-free nucleic acid in nucleic acid is of a length of about 200 base pairs or less (e.g., about 80, 85, 90, 91, 92, 93, 94, 95, 96, 97, 98, 99 or 100% of fetus-specific nucleic acid is of a length of about 200 base pairs or less). In some embodiments, the majority of fetus-specific cell-free nucleic acid in nucleic acid is of a length of about 150 base pairs or less (e.g., about 80, 85, 90, 91, 92, 93, 94, 95, 96, 97, 98, 99 or 100% of fetus-specific cell-free nucleic acid is of a length of about 150 base pairs or less). In some embodiments, the majority of fetus-specific cell-free nucleic acid is of a length of about 100 base pairs or less (e.g., about 80, 85, 90, 91, 92, 93, 94, 95, 96, 97, 98, 99 or 100% of fetus-specific nucleic acid is of a length of about 100 base pairs or less).

Methods for isolating cell-free DNA from liquid biological samples, such as blood or serum samples, are well known. In one illustrative example, magnetic beads are used to bind the cfDNA and then bead-bound cfDNA is washed and eluted from the magnetic beads. An exemplary method of isolating cell-free DNA is described in WO2017074926, the entire content of which is hereby incorporated by reference. Commercial kits for isolating cell free DNA are also available, for example, MagNA Pure Compact (MPC) Nucleic Acid Isolation Kit I, Maxwell RSC (MR) ccfDNA Plasma Kit, the QIAamp Circulating Nucleic Acid (QCNA) kit.

In some cases, the cell-free nucleic acids may be isolated from samples obtained at a different time points of pregnancy. The fetal-specific allele frequencies and genotypes are determined for each of the time points as decribed above, and a comparison between the time points can often confirm fetal genotypes. A nucleic acid may be a result of nucleic acid purification or isolation and/or amplification of nucleic acid molecules from the sample. Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples). In some embodiments, the pooled samples may be from the same patient, e.g., pregnant mother, but are taken at different time points, or are of different tissue type. In some embodients, the pooled samples may be from different patients. As described further below, in some embodiments, identifiers are attached to the nucleic acids derived from the each of the one or more samples to distinguish the sources of the sample.

Nucleic acid may be provided for conducting methods described herein without processing of the sample(s) containing the nucleic acid, in certain embodiments. In some embodiments, nucleic acid is provided for conducting methods described herein after processing of the sample(s) containing the nucleic acid. For example, a nucleic acid may be extracted, isolated, purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. An isolated nucleic acid is provided with fewer non-nucleic acid components (e.g., protein, lipid) than the amount of components present in a source sample. A composition comprising isolated nucleic acid can be about 90%, 91%, 92%, 93%, 94%, 95%, 96%, 97%, 98%, 99% or greater than 99% free of non-nucleic acid components. The term “purified” as used herein refers to nucleic acid provided that contains fewer nucleic acid species than in the sample source from which the nucleic acid is derived. A composition comprising nucleic acid may be about 90%, 91%, 92%, 93%, 94%, 95%, 96%, 97%, 98%, 99% or greater than 99% free of other nucleic acid species. The term “amplified” as used herein refers to subjecting nucleic acid of a sample to a process that linearly or exponentially generates amplicon nucleic acids having the same or substantially the same nucleotide sequence as the nucleotide sequence of the nucleic acid in the sample, or portion thereof.

Nucleic acid may be single or double stranded. Single stranded DNA, for example, can be generated by denaturing double stranded DNA by heating or by treatment with alkali, for example. In some cases, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art. In some cases nucleic acids may be fragmented using either physical or enzymatic methods known in the art.

### DNA Target Sequences

In some embodiments of the methods provided herein, one or more nucleic acid species, and sometimes one or more nucleotide sequence species, are targeted for amplification and quantification. In some embodiments, the targeted nucleic acids are genomic DNA sequences. Certain DNA target sequences are used, for example, because they can allow for the determination of a particular feature for a given assay. DNA target sequences can be referred to herein as markers for a given assay. In some cases, target sequences are polymorphic, for example, one or more SNVs as described herein. In some embodiments, more than one DNA target sequence or marker can allow for the determination of a particular feature for a given assay. Such genomic DNA target sequences are considered to be of a particular “region”. As used herein, a “region” is not intended to be limited to a description of a genomic location, such as a particular chromosome, stretch of chromosomal DNA or genetic locus. Rather, the term “region” is used herein to identify a collection of one or more genomic DNA target sequences or markers that can be indicative of a particular assay. Such assays can include, but are not limited to, assays for the detection and quantification of fetus-specific nucleic acid, assays for the detection and quantification of maternal nucleic acid, assays for the detection and quantification of total DNA, assays for the detection and quantification of methylated DNA, assays for the detection and quantification of DNA from one or more potential fathers, and assays for the detection and quantification of digested and/or undigested DNA, as an indicator of digestion efficiency. In some embodiments, the genomic DNA target sequence is described as being within a particular genomic locus. As used herein, a genomic locus can include any or a combination of open reading frame DNA, non-transcribed DNA, intronic sequences, extronic sequences, promoter sequences, enhancer sequences, flanking sequences, or any sequences considered by one of skill in the art to be associated with a given genomic locus.

In some embodiments, the sample may first be enriched or relatively enriched for fetus-specific nucleic acid by one or more methods. For example, the discrimination of fetal and maternal DNA can be performed using the compositions and processes of the present technology alone or in combination with other discriminating factors. Examples of these factors include, but are not limited to, single nucleotide differences between polymorphisms located in the genome.

Other methods for enriching a sample for a particular species of nucleic acid are described in PCT Patent Application Number PCT/US07/69991, filed May 30, 2007, PCT Patent Application Number PCT/US2007/071232, filed Jun. 15, 2007, U.S. Provisional Application Nos. 60/968,876 and 60/968,878 (assigned to the Applicant), (PCT Patent Application Number PCT/EP05/012707, filed Nov. 28, 2005) which are all hereby incorporated by reference. In certain embodiments, recipient nucleic acid is selectively removed (either partially, substantially, almost completely or completely) from the sample.

### Methods for Determining Fetus-Specific Cell-Free Nucleic Acid Content

In some embodiments, the amount of fetus-specific cell free nucleic acids in a sample is determined. In some cases, the amount of fetus-specific nucleic acid is determined based on a quantification of sequence read counts described herein. Quantification may be achieved by direct counting of sequence reads covering particular target sites, or by competitive PCR (i.e., co-amplification of competitor oligonucleotides of known quantity, as described herein). The term “amount” as used herein with respect to nucleic acids refers to any suitable measurement, including, but not limited to, absolute amount (e.g. copy number), relative amount (e.g. fraction or ratio), weight (e.g., grams), and concentration (e.g., grams per unit volume (e.g., milliliter); molar units). As used herein, when an action such as a determination of something is “triggered by”, “according to”, or “based on” something, this means the action is triggered, according to, or based at least in part on at least a part of the something.

In some embodiments, the relative amount or the proportion of fetus-specific cell-free nucleic acid is determined according to allelic ratios of polymorphic sequences, or according to one or more markers specific to fetus-specific nucleic acid and not maternal nucleic acid. In some cases, the amount of fetus-specific cell-free nucleic acid relative to the total cell-free nucleic acid in a sample is referred to as “fetus-specific nucleic acid fraction”.

### Polymorphism-Based Donor Quantifier Assay

Determination of fetus-specific nucleic acid content (e.g., fetus-specific nucleic acid fraction) sometimes is performed using a polymorphism-based fetus quantifier assay, as described herein. This type of assay allows for the detection and quantification of fetus-specific nucleic acid in a sample from a pregnant mother based on allelic ratios of polymorphic nucleic acid target sequences (e.g., single nucleotide variants (SNVs)).

In some cases, fetus-specific alleles are identified, for example, by their relative minor contribution to the mixture of fetal and maternal cell-free nucleic acids in the sample when compared to the major contribution to the mixture by the maternal nucleic acids. In some cases, fetus-specific alleles are identified by a deviation of the measured allele frequency in the total cell-free nucleic acids from an expected allele frequency, as described below. In some cases, the relative amount of fetus-specific cell-free nucleic acid in a maternal sample can be determined as a parameter of the total number of unique sequence reads mapped to a target nucleic acid sequence on a reference genome for each of the two alleles (a reference allele and an alternate allele) of a polymorphic site. In some cases, the relative amount of fetus-specific cell-free nucleic acid in a maternal sample can be determined as a parameter of the relative number of sequence reads for each allele from an enriched sample.

### Selecting Polymorphic Nucleic Ncid Targets

In some embodiments, the polymorphic nucleic acid targets are one or more of a: (i) single nucleotide variant (SNV); (ii) insertion/deletion polymorphism, (iii) restriction fragment length polymorphism (RFLPs), (iv) short tandem repeat (STR), (v) variable number of tandem repeats (VNTR), (vi) a copy number variant, (vii) an insertion/deletion variant, or (viii) a combination of any of (i)-(vii) thereof.

A polymorphic marker or site is the locus at which divergence occurs. Polymorphic forms also are manifested as different alleles for a gene. In some embodiments, there are two alleles for a polymorphic nucleic acid target and these polymorphic nucleic acid targets are called biallelic polymorphic nucleic acid targets. In some embodiments, there are three, four, or more alleles for a polymorphic nucleic acid target.

In some embodiments, one of these alleles is referred to as a reference allele and the others are referred to as alternate alleles. Polymorphisms can be observed by differences in proteins, protein modifications, RNA expression modification, DNA and RNA methylation, regulatory factors that alter gene expression and DNA replication, and any other manifestation of alterations in genomic nucleic acid or organelle nucleic acids.

Numerous genes have polymorphic regions. Since individuals have any one of several allelic variants of a polymorphic region, individuals can be identified based on the type of allelic variants of polymorphic regions of genes. This can be used, for example, for forensic purposes or for identifying familial relationships. For example, the paternity of a fetus (i.e., identity of the paternal parent of origin or father) can be determined by comparing allelic variants of the fetus to those of one or more potential fathers. In other situations, it is crucial to know the identity of allelic variants that an individual has. For example, allelic differences in certain genes, for example, major histocompatibility complex (MHC) genes, are involved in graft rejection or graft versus host disease in bone marrow transplantation. Accordingly, it is highly desirable to develop rapid, sensitive, and accurate methods for determining the identity of allelic variants of polymorphic regions of genes or genetic lesions.

In some embodiments, the polymorphic nucleic acid targets are single nucleotide variants (SNVs). Single nucleotide variants (SNVs) are generally biallelic systems, that is, there are two alleles that an individual can have for any particular marker, one of which is referred to as a reference allele and the other referred to as an alternate allele. This means that the information content per SNV marker is relatively low when compared to microsatellite markers, which can have upwards of 10 alleles. SNVs also tend to be very population-specific; a marker that is polymorphic in one population sometimes is not very polymorphic in another. SNVs, found approximately every kilobase (see Wang et al. (1998) Science 280: 1077-1082), offer the potential for generating very high density genetic maps, which will be extremely useful for developing haplotyping systems for genes or regions of interest, and because of the nature of SNVs, they can in fact be the polymorphisms associated with the disease phenotypes under study. The low mutation rate of SNVs also makes them excellent markers for studying complex genetic traits.

Much of the focus of genomics has been on the identification of SNVs, which are important for a variety of reasons. SNVs allow indirect testing (association of haplotypes) and direct testing (functional variants). SNVs are the most abundant and stable genetic markers. Common diseases are best explained by common genetic alterations, and the natural variation in the human population aids in understanding disease, therapy and environmental interactions.

In some embodiments, the polymorphic nucleic acid marker targets comprises at least one, two, three, four or more SNVs in Table 1 or Table 5. These SNVs have alternative alleles occurring frequently in individuals within a population. As well, these SNVs are diverse and present in multiple populations. Informative analysis indicates that possibility to design specific nucleic acid primers to these SNVs with low potential for off-target non-specific amplification.

TABLE 1

Exemplary SNVs

| | |
|---|---|
| Panel A | rs10737900, rs1152991, rs10914803, rs4262533, rs686106, rs3118058, rs4147830, rs12036496, rs 1281182, rs863368, rs765772, rs6664967, rs 12045804, rs1160530, rs11119883, rs751128, rs7519121, rs9432040, rs7520974, rs1879744, rs6739182, rs4074280, rs7608890, rs6758291, rs13026162, rs2863205, rs11126021, rs9678488, rs10168354, rs13383149, rs955105, rs2377442, rs13019275, rs967252, rs16843261, rs2049711, rs2389557, rs6434981, rs1821662, rs1563127, rs7422573, rs6802060, rs9879945, rs7652856, rs1030842, rs614004, rs1456078, rs6599229, rs1795321, rs4928005, rs9870523, rs7612860, rs11925057, rs792835, rs9867153, rs602763, rs12630707, rs2713575, rs9682157, rs13095064, rs2622744, rs12635131, rs7650361, rs16864316, rs9810320, rs9841174, rs7626686, rs9864296, rs2377769, rs4687051, rs1510900, rs6788448, rs11941814, rs4696758, rs7440228, rs13145150, rs17520130, rs11733857, rs6828639, rs6834618, rs16996144, rs376293, rs11098234, rs975405, rs1346065, rs1992695, rs6849151, rs11099924, rs6857155, rs10033133, rs7673939, rs7700025, rs6850094, rs11132383, rs7716587, rs38062, rs582991, rs2388129, rs9293030, rs11738080, rs13171234, rs309622, rs253229, rs11744596, rs4703730, rs10040600, rs11953653, rs163446, rs4920944, rs11134897, rs226447, rs12194118, rs4959364, rs4712253, rs2457322, rs7767910, rs2814122, rs6930785, rs1145814, rs1341111, rs2615519, rs1894642, rs6570404, rs9479877, rs9397828, rs6927758, rs6461264, rs6947796, rs1347879, rs10246622, rs10232758, rs756668, rs2709480, rs1983496, rs1665105, rs11785007, rs10089460, rs1390028, rs4738223, rs6981577, rs10958016, rs9298424, rs517811, rs1442330, rs1002142, rs2922446, rs1514221, rs387413, rs10758875, rs10759102, rs2183830, rs1566838, rs12553648, rs10781432, rs11141878, rs2756921, rs1885968, rs10980011, rs1002607, rs10987505, rs1334722, rs723211, rs4335444, rs7917095, rs10509211, rs10881838, rs2286732, rs4980204, rs12286769, rs4282978, rs7112050, rs7932189, rs7124405, rs7111400, rs1938985, rs7925970, rs7104748, rs10790402, rs2509616, rs4609618, rs12321766, rs2920833, rs10133739, rs10134053, rs7159423, rs2064929, rs1298730, rs2400749, rs12902281, rs11074843, rs9924912, rs1562109, rs2051985, rs8067791, rs12603144, rs16950913, rs1486748, rs2570054, rs2215006, rs4076588, rs7229946, rs9945902, rs1893691, rs930189, rs3745009, rs1646594, rs7254596, rs511654, rs427982, rs10518271, rs1452321, rs6080070, rs6075517, rs6075728, rs6023939, rs3092601, rs6069767, rs2426800, rs2826676, rs2251381, rs2833579, rs1981392, rs1399591, rs2838046, rs8130292, rs241713 |
| Panel B | rs10413687, rs10949838, rs1115649, rs11207002, rs11632601, rs11971741, rs12660563, rs13155942, rs1444647, rs1572801, rs17773922, rs1797700, rs1921681, rs1958312, rs196008, rs2001778, rs2323659, rs2427099, rs243992, rs251344, rs254264, rs2827530, rs290387 , rs321949, rs348971, rs390316, rs3944117, rs425002, rs432586, rs444016, rs4453265, rs447247, rs4745577, rs484312, rs499946, rs500090, rs500399, rs505349, rs505662 , rs516084, rs517316, rs517914, rs522810, rs531423, rs537330, rs539344, rs551372, rs567681, rs585487, rs600933, rs619208, rs622994, rs639298, rs642449, rs6700732, rs677866, rs683922, rs686851, rs6941942, rs7045684, rs7176924, rs7525374, rs870429, rs949312, rs9563831 , rs970022, rs985462, rs1005241, rs1006101, rs10745725, rs10776856, rs10790342, rs11076499, rs11103233, rs11133637, rs11974817, rs12102203, rs12261, rs12460763, rs12543040, rs12695642, rs13137088, rs13139573, rs1327501, rs13438255, rs1360258, rs1421062, rs1432515, rs1452396, rs1518040, rs16853186, rs1712497, rs1792205, rs1863452, rs1991899, rs2022958, rs2099875, rs2108825, rs2132237, rs2195979, rs2248173, rs2250246, rs2268697, rs2270893, rs244887, rs2736966, rs2851428, rs2906237, rs2929724, rs3742257, rs3764584, rs3814332, rs4131376, rs4363444, rs4461567, rs4467511, rs4559013, rs4714802, rs4775899, rs4817609, rs488446, rs4950877, rs530913, rs6020434, rs6442703, rs6487229, rs6537064, rs654065, rs6576533, rs6661105, rs669161, rs6703320, rs675828, rs6814242, rs6989344, rs7120590, rs7131676, rs7214164, rs747583, rs768255, rs768708, rs7828904, rs7899772, rs7900911, rs7925270, rs7975781, rs8111589, rs849084, rs873870, rs9386151, rs9504197, rs9690525, rs9909561, rs10839598, rs10875295, rs12102760, rs12335000, rs12346725, rs12579042, rs12582518, rs17167582, rs1857671, rs2027963, rs2037921, rs2074292, rs2662800, rs2682920, rs2695572, rs2713594, rs2838361, rs315113, rs3735114, rs3784607, rs3817, rs3850890, rs3934591, rs4027384, rs405667, rs4263667, rs4328036, rs4399565, rs4739272, rs4750494, rs4790519, rs4805406, rs4815533, rs483081, rs4940791, rs4948196, rs582111, rs596868, rs6010063, rs6014601, rs6050798, rs6131030, rs631691, rs6439563, rs6554199, rs6585677, rs6682717, rs6720135, rs6727055, rs6744219, rs6768281, rs681836, rs6940141, rs6974834, rs718464, rs7222829, rs7310931, rs732478, rs7422573, rs7639145, rs7738073, rs7844900, rs7997656, rs8069699, rs8078223, rs8080167, rs8103778, rs8128, rs8191288, rs886984, rs896511, rs931885, rs9426840, rs9920714, rs9976123, rs999557, rs9997674 |

In some embodiments, the polymorphic nucleic acid targets selected for determining paternity are a combination of any of the polymorphic nucleic acid targets in Table 1 (Panel A, and/or panel B) or Table 5.

A plurality of polymorphic nucleic acid targets is sometimes referred to as a collection or a panel (e.g., target panel, SNV panel, SNV collection). In some cases, the panel include 2-1000 polymorphic nucleic acid targets, e.g., 10 to 1000, 50 to 800, or 100 to 500, or 150 to 300. A plurality of polymorphic targets can comprise two or more targets. For example, a plurality of polymorphic targets can comprise 2, 3, 4, 5, 6, 7, 8, 9, 10, 20, 30, 40, 50, 60, 70, 80, 90, 100, 200, 300, 400, 500, 600, 700, 800, 900, 1000, or more targets.

In some cases, 10 or more polymorphic nucleic acid targets are enriched using the methods described herein. In some cases, 50 or more polymorphic nucleic acid targets are enriched. In some cases, 100 or more polymorphic nucleic acid targets are enriched. In some cases, 500 or more polymorphic nucleic acid targets are enriched. In some cases, about 10 to about 500 polymorphic nucleic acid targets are enriched. In some cases, about 20 to about 400 polymorphic nucleic acid targets are enriched. In some cases, about 30 to about 200 polymorphic nucleic acid targets are enriched. In some cases, about 40 to about 100 polymorphic nucleic acid targets are enriched. In some cases, about 60 to about 90 polymorphic nucleic acid targets are enriched. For example, in certain embodiments, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 polymorphic nucleic acid targets are enriched.

### Identifying the Informative Polymorphic Nucleic Acid Targets

In some embodiments, at least one polymorphic nucleic acid target of the plurality of polymorphic nucleic acid targets is informative for determining fetus-specific nucleic acid fraction and/or paternity in a given sample. A polymorphic nucleic acid target that is informative for determining fetus-specific nucleic acid fraction and/or paternity, sometimes referred to as an informative target or an informative polymorphism (e.g., informative SNV), typically differs in some aspect between the fetus and the mother. For example, an informative target may have one allele for the fetus and a different allele for the mother (e.g., the mother has allele A at the polymorphic target and the fetus has allele B at the polymorphic target site).

In some cases, polymorphic nucleic acid targets are informative in the context of certain fetus/mother genotype combinations. For a biallelic polymorphic target (i.e., two possible alleles (e.g., A and B, wherein A is a reference allele and B is an alternate allele, or vice versa)), possible fetus/mother genotype combinations include: 1) mother AA, fetus AA; 2) mother AA, fetus AB; 3) mother AB, fetus AA; 4) mother AB, fetus AB; 5) mother AB; fetus BB; 6) mother BB, fetus AB; and 7) mother BB, fetus BB. In some cases, informative genotype combinations (i.e., genotype combinations for a polymorphic nucleic acid target that may be informative for determining fetus-specific nucleic acid fraction and/or paternity) include combinations where the mother is homozygous and the fetus is heterozygous (e.g., mother AA, fetus AB; or mother BB, fetus AB). Such genotype combinations may be referred to as Type 1 informative genotypes. In some cases, informative genotype combinations (i.e., genotype combinations for a polymorphic nucleic acid target that may be informative for determining fetus-specific nucleic acid fraction and/or paternity) include combinations where the mother is heterozygous and the fetus is homozygous (e.g., mother AB, fetus AA; or mother AB, fetus BB). Such genotype combinations may be referred to as Type 2 informative genotypes. In some cases, non-informative genotype combinations (i.e., genotype combinations for a polymorphic nucleic acid target that may not be informative for determining fetus-specific nucleic acid fraction and/or paternity) include combinations where the mother is heterozygous and the fetus is heterozygous (e.g., mother AB, fetus AB). Such genotype combinations may be referred to as non-informative genotypes or non-informative heterozygotes. In some cases, non-informative genotype combinations (i.e., genotype combinations for a polymorphic nucleic acid target that may not be informative for determining fetus-specific nucleic acid fraction and/or paternity) include combinations where the mother is homozygous and the fetus is homozygous (e.g., mother AA, fetus AA; or mother BB, fetus BB). Such genotype combinations may be referred to as non-informative genotypes or non-informative homozygotes. In some embodiments, the mother’s genotype for the polymorphic nucleic acid targets is determined prior to pregnancy. In some embodiments, the mother’s genotype for the polymorphic nucleic acid targets is determined from samples which do not comprise fetal nucleic acids (e.g., nucleic acids derived from blood buffy coat fraction, or buccal swab samples, as described herein). The presence of fetus-specific cell-free nucleic acids can be readily determined by selecting the informative polymorphic nucleic acid targets as described above, and detecting and/or quantifying the fetus-specific alleles of the polymorphic nucleic acid targets using the assays described herein.

In some embodiments, individual polymorphic nucleic acid targets and/or panels of polymorphic nucleic acid targets are selected based on certain criteria, such as, for example, minor allele frequency, variance, coefficient of variance, MAD value, and the like. In some cases, polymorphic nucleic acid targets are selected so that at least one polymorphic nucleic acid target within a panel of polymorphic targets has a high probability of being informative for a majority of samples tested. Additionally, in some cases, the number of polymorphic nucleic acid targets (i.e., number of targets in a panel) is selected so that least one polymorphic nucleic acid target has a high probability of being informative for a majority of samples tested. For example, selection of a larger number of polymorphic targets generally increases the probability that least one polymorphic nucleic acid target will be informative for a majority of samples tested. In some cases, the polymorphic nucleic acid targets and number thereof (e.g., number of polymorphic targets selected for enrichment) result in at least about 2 to about 50 or more polymorphic nucleic acid targets being informative for determining the fetus-specific nucleic acid fraction and/or paternity for at least about 80% to about 100% of samples. For example, the polymorphic nucleic acid targets and number thereof result in at least about 5, 10, 15, 20, 25, 30, 35, 40, 45, 50 or more polymorphic nucleic acid targets being informative for determining the fetus-specific nucleic acid fraction and/or paternity for at least about 81%, 82%, 83%, 84%, 85%, 86%, 87%, 88%, 89%, 90%, 91%, 92%, 93%, 94%, 95%, 96%, 97%, 98%, or 99% of samples. In some cases, the polymorphic nucleic acid targets and number thereof result in at least five polymorphic nucleic acid targets being informative for determining the fetus-specific nucleic acid fraction and/or paternity for at least 90% of samples. In some cases, the polymorphic nucleic acid targets and number thereof result in at least five polymorphic nucleic acid targets being informative for determining the fetus-specific nucleic acid fraction and/or paternity for at least 95% of samples. In some cases, the polymorphic nucleic acid targets and number thereof result in at least five polymorphic nucleic acid targets being informative for determining the fetus-specific nucleic acid fraction and/or paternity for at least 99% of samples. In some cases, the polymorphic nucleic acid targets and number thereof result in at least ten polymorphic nucleic acid targets being informative for determining the fetus-specific nucleic acid fraction and/or paternity for at least 90% of samples. In some cases, the polymorphic nucleic acid targets and number thereof result in at least ten polymorphic nucleic acid targets being informative for determining the fetus-specific nucleic acid fraction and/or paternity for at least 95% of samples. In some cases, the polymorphic nucleic acid targets and number thereof result in at least ten polymorphic nucleic acid targets being informative for determining the fetus-specific nucleic acid fraction and/or paternity for at least 99% of samples.

In some embodiments, individual polymorphic nucleic acid targets are selected based, in part, on minor allele frequency. In some cases, polymorphic nucleic acid targets having minor allele frequencies of about 10% to about 50% are selected. For example, polymorphic nucleic acid targets having minor allele frequencies that ranges between 15-49%, e.g., 20-49%, 25-45%, 35-49%, or 40-40%. In some embodiments, the polymorphic nucleic acid target has a minor allele allele frequency of about 15%, 20%, 25%, 30%, 35%, 36%, 37%, 38%, 39%, 40%, 41%, 42%, 43%, 44%, 45%, 46%, 47%, 48%, or 49% are selected. In some embodiments, polymorphic nucleic acid targets having a minor allele frequency of about 40% or more are selected. In some cases, the minor allele frequencies of the polymorphic nucleic acid targets can be identified from published databases or based on study results from a reference population.

By analyzing a panel of multiple polymorphic nucleic acid targets (e.g., SNVs) (for instance on the order of 100, 200, 300, etc.) with high minor allele frequencies (for instance from 0.4-0.5), a significant number of ‘informative’ fetal and maternal genotype combinations (with fetal genotypes differing from mother’s genotype) may be seen. In some embodiments, the number of the polymorphic nucleic acid targets that in the panel is in the range of between 20 and 10,000, e.g., between 30 and 5000, between 50 and 950, between 100 and 500, between 150 and 400, or between 200 and 350, from which informative polymorphic nucleic acid targets can be determined using the methods disclosed herein. In some embodiments, polymorphic nucleic acid targets of the type 1 Informative genotypes, where the mother is homozygous for one allele and the fetus is heterozygous, are used to determine a change in allele frequency due to the minimal impact of molecular sampling error on the background mother homozygous allele frequency. In some embodiments, about 25% of the polymorphic nucleic acid targets in a panel are informative where the mother is homozygous for one reference allele or one alternate allele and the fetus is heterozygous.

In some embodiments, the polymorphic nucleic acid targets are selected based on the GC content of the region surrounding the polymorphic nucleic acid targets and the amplification efficiency of the polymorphic nucleic acid targets. In some embodiments, the GC content is in a range of 10% to 80%, e.g., 20% to 70%, or 25% to 70%, 21% to 61% or 30% to 61%.

In some embodiments, individual polymorphic nucleic acid targets and/or panels of polymorphic nucleic acid targets are selected based, in part, on degree of variance for an individual polymorphic target or a panel of polymorphic targets. Variance, in some cases, can be specific for certain polymorphic targets or panels of polymorphic targets and can be from systematic, experimental, procedural, and or inherent errors or biases (e.g., sampling errors, sequencing errors, PCR bias, and the like). Variance of an individual polymorphic target or a panel of polymorphic targets can be determined by any method known in the art for assessing variance and may be expressed, for example, in terms of a calculated variance, an error, standard deviation, p-value, mean absolute deviation, median absolute deviation, median adjusted deviation (MAD score), coefficient of variance (CV), and the like. In some embodiments, measured allele frequency variance (i.e., background allele frequency) for certain SNVs (when homozygous, for example) can be from about 0.001 to about 0.01 (i.e., 0.1% to about 1.0%). For example, measured allele frequency variance can be about 0.002, 0.003, 0.004, 0.005, 0.006, 0.007, 0.008, or 0.009. In some cases, measured allele frequency variance is about 0.007.

In some cases, noisy polymorphic targets are excluded from a panel of polymorphic nucleic acid targets selected for determining fetus-specific nucleic acid fraction and/or paternity. The term “noisy polymorphic targets” or “noisy SNVs” refers to (a) targets or SNVs that have significant variance between data points (e.g., measured fetus-specific nucleic acid fraction, measured allele frequency) when analyzed or plotted, (b) targets or SNVs that have significant standard deviation (e.g., greater than 1, 2, or 3 standard deviations), (c) targets or SNVs that have a significant standard error of the mean, the like, and combinations of the foregoing. Noise for certain polymorphic targets or SNVs sometimes occurs due to the quantity and/or quality of starting material (e.g., nucleic acid sample), sometimes occurs as part of processes for preparing or replicating DNA used to generate sequence reads, and sometimes occurs as part of a sequencing process. In certain embodiments, noise for some polymorphic targets or SNVs results from certain sequences being over represented when prepared using PCR-based methods. In some cases, noise for some polymorphic targets or SNVs results from one or more inherent characteristics of the site such as, for example, certain nucleotide sequences and/or base compositions surrounding, or being adjacent to, a polymorphic target or SNV. A SNV having a measured allele frequency variance (when homozygous, for example) of about 0.005 or more may be considered noisy. For example, a SNV having a measured allele frequency variance of about 0.006, 0.007, 0.008, 0.009, 0.01 or more may be considered noisy.

In some embodiments, the reference allele and alternate allele combination of one or more SNVs selected for determining the paternity is not any one of A_G, G_A, C_T, and T_C (the first letter refers to the reference allele and the second letter refers to the alternate allele). As shown in FIG. 8 and Example 2, SNVs having the above reference allele and alternate allele combination showed higher amount of bias and variability and thus they are not suitable for use in the method disclosed herein for determining the fetal fraction and/or paternity.

In some embodiments, the one or more SNVs selected for determining paternity meet one or more, or all of the following criteria:
1. Biallelic.
2. The SNV is not located within the primer annealing regions.
3. Validated by the 1000 Genomes Project.
4. The ref_{_}alt combination is not any of the A_G, G_A, C_T or T_C.
5. Minor allele frequency is at least 0.3.
6. The sequence for amplified target region is unique and cannot be found elsewhere in the genome.

In some embodiments, variance of an individual polymorphic target or a panel of polymorphic targets can be represented using coefficient of variance (CV). Coefficient of variance (i.e., standard deviation divided by the mean) can be determined, for example, by determining fetus-specific nucleic acid fraction for several aliquots of a single maternal sample comprising mother-specific and fetus-specific nucleic acid, and calculating the mean fetus-specific nucleic acid fraction and standard deviation. In some cases, individual polymorphic nucleic acid targets and/or panels of polymorphic nucleic acid targets are selected so that fetus-specific nucleic acid fraction is determined with a coefficient of variance (CV) of 0.30 or less. For example, fetus-specific nucleic acid fraction may be determined with a coefficient of variance (CV) of 0.25, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01 or less, in some embodiments. In some cases, fetus-specific nucleic acid fraction is determined with a coefficient of variance (CV) of 0.20 or less. In some cases, fetus-specific nucleic acid fraction is determined with a coefficient of variance (CV) of 0.10 or less. In some cases, fetus-specific nucleic acid fraction is determined with a coefficient of variance (CV) of 0.05 or less.

In some embodiments, an allele frequency is determined for one or more alleles of the polymorphic nucleic acid targets in a sample. This sometimes is referred to as measured allele frequency. Allele frequency can be determined, for example, by counting the number of sequence reads for an allele (e.g., allele B) and dividing by the total number of sequence reads for that locus (e.g., allele B + allele A). In some cases, an allele frequency average, mean or median is determined. In some cases, fetus-specific nucleic acid fraction can be determined based on the allele frequency mean (e.g., allele frequency mean multiplied by two).

In some embodiments, quantification data (e.g., sequencing data) covering the polymorphic nucleic acid target are used to count the number of times the genomic positions of the polymorphic nucleic acid target (e.g., an SNV) are sequenced. The number of sequencing reads containing the reference allele and the alternate allele of the polymorphic nucleic acid target, respectively, can be determined. For example, in a sample homozygous for the reference allele of a SNV, there would ideally be a reference SNV allele frequency of about 1.0 (e.g. 0.99-1.00) where all sequencing reads covering the SNV contain the reference SNV allele. When the sample is heterozygous for both the reference and alternate allele, the expected allele frequency for the reference SNV allele is about 0.5 (e.g., 0.46-0.53). When the sample is homozygous for the alternate allele, the expected reference SNV allele frequency would be 0. These values of 1.0, 0.5, and 0 are idealized, however, and while measurements will generally approach these values, real-world SNV allele frequency measurement will be influenced by biochemical, sequencing, and process error. In the case of heterozygous allele frequencies, these will also be influenced by molecular sampling error.

In some embodiments, the mother’s genotype is determined separately from a genomic DNA sample (e.g., from buffy coat fraction as described above) during or before pregnancy, and the presence of fetus-specific alleles can be readily detected and quantified. However, in some cases, genotyping the mother may not be possible due to the lack of a genomic DNA sample. In some cases, the mother’s genotype for one or more polymorphic targets is not determined before paternity determination. In some embodiments, this disclosure provides methods and systems that can be used to detect and/or quantify fetus-specific cell free nucleic acids even in the absence of the mother’s genotype information. This can be advantageous in situations where the patient is not submitted to testing until during pregnancy, at which point no prepregnancy samples from the mother are accessible for genotyping. Dispensing the need for genotyping before pregnancy also saves costs in tracking the patient information. Without being bound to a particular theory, the present invention can determine the mother’s genotype during pregnancy from a mixture that includes both fetal and maternal cell-free DNA from samples taken during pregnancy. This is based on the fact that each of the SNVs allele frequencies before pregnancy will cluster around heterozygous (0.5) or homozygous (0 or 1). When there is a difference in fetal and maternal genotype, there’ll be a deviation (proportional to the fetal fraction) from heterozygous or homozygous. When there is a match in fetal and maternal genotype, the allele frequency in the mixed cell-free DNA will be the same as the allele frequency in the genotype of the mother before pregnancy. These two categories of maternal-fetal genotype combinations are further illustrated below.

Fetal and maternal genotypes are different (results in a fetus-specific deviation of the allele frequency):
AAₘₒₜₕₑᵣ/AB_{fetus}
ABₘₒₜₕₑᵣ/AA_{fetus}
ABₘₒₜₕₑᵣ/BB_{fetus}
BBₘₒₜₕₑᵣ/AB_{fetus}

Fetal and maternal genotypes are the same (so the resulting allele frequency is the “expected” maternal genotype):
AAₘₒₜₕₑᵣ/AA_{fetus}
ABₘₒₜₕₑᵣ/AB_{fetus}
BBₘₒₜₕₑᵣ/BB_{fetus}

(A represents the reference allele and B represents the alternate allele.)

The deviation is the difference between the allele frequency in the cell free DNA sample from the mother where the fetal genotype matches with the maternal genotype (i.e., the expected allele frequency) and the allele frequency in the cell free DNA sample where the fetal genotype does not match the maternal genotype (i.e., the measured allele frequency). In some cases, an allele frequency average, mean or median is determined for the expected allele frequency and measured allele frequency and used for calculation of the deviation.

Thus, for SNVs where the mother is homozygous for the alternate allele (the reference allele frequency is about 0, or is in the range of 0.00-0.03, 0.00-0.02, e.g., 0.00-0.01), the deviation is the difference in mean or median of allele frequencies where the fetus is homozygous for the alternate allele (matching maternal genotype) vs. the mean or median of allele frequencies where the fetus is either heterozygous or homozygous for the reference allele (differing from maternal genotype).

For SNVs where the mother is heterozygous for the alternate allele (the reference allele frequency is about 0.5, or is in the range of 0.40-0.60, 0.42-0.56, or 0.46-0.53), the deviation is the difference in mean or median of allele frequencies where the fetus is heterozygous for the alternate allele (matching maternal genotype) vs. the mean or median of allele frequencies where the fetus is either homozygous for the alternate allele or homozygous for the reference allele (differing form maternal genotype).

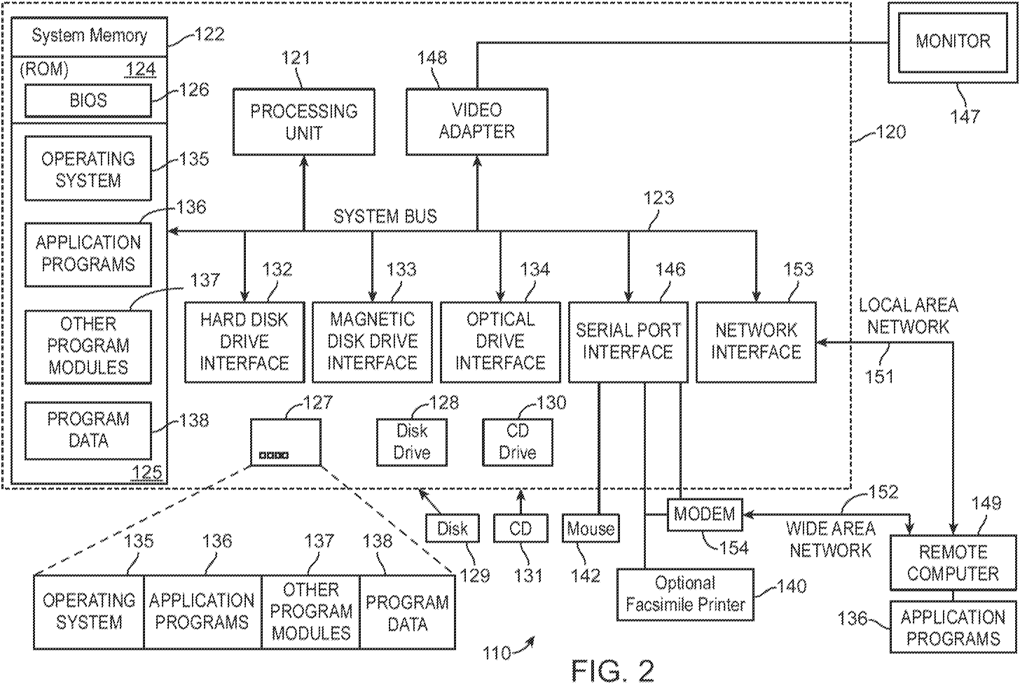

For SNVs where the mother is homozygous for the reference allele (the reference allele frequency is about 1.00, or in the range of 0.97-1.00, or 0.98-1.00, e.g., 0.99-1.00), the deviation is the difference in mean or median of allele frequencies where the fetus is homozygous for the reference allele (matching maternal genotype) vs. the mean or median of allele frequencies where the fetus is either heterozygous or homozygous for the alternate allele (differing form maternal genotype). Whether a particular fetus/mother genotype combination belongs to one or another category can be determined based on a single sample comprising a mixture of maternal and fetal DNA, without genotyping the fetus or genotyping the mother before pregnancy by using the methods as described below. In these cases, these methods assume that normal SNV allele frequencies (allele frequencies associated with homozygous alternate allele genotypes, heterozygous alternate and reference allele genotypes, or homozygous reference allele genotypes) are present from the allele background of the mother. In these cases, the fetus-specific nucleic acids can be identified using, for example, one or more of a fixed cutoff approach, a dynamic clustering approach, and an individual polymorphic nucleic acid target threshold approach, as described below. Table 2 shows the features of the various exemplary approaches that can be used for these purposes. Such approaches may be performed by a processor, a micro-proccesor, a computer system, in conjunction with memory and/or by a microprocessor controlled apparatus. In various embodiments, the approaches are performed as a sequence of events or steps (e.g., a method or process) in the operating environment 110 described with respect to FIG. 2 herein.

TABLE 2

| Methods | Description |
|---|---|
| Quality filtering of sequencing reads | Monitor and filter sequence read quality scores with exclusion of low quality sequence reads, |
| | Decreases background noise in SNV allele frequency measurement |
| | Does not contribute directly to detection of fetal alleles, but will enable a more precise genotype frequency calculation |
| Fixed cutoff for homozygous variance | Establish a fixed cutoff level for homozygous allele frequencies defined as a fixed percentile of homozygous SNV allele frequencies |
| | Easily established by analysis of a moderate sized cohort |
| | Does not allow for differences in variance across SNVs within a panel |
| Dynamic k-means clustering | Use clustering algorithm (k-means) on a per sample basis |
| | Two-tiered approach to dynamically stratify SNVs based on maternal homozygous or heterozygous genotype and then stratify maternal homozygous SNVs into non-informative and informative groups |
| SNV specific variance threshold | ·Establish specific homozygous allele frequencies threshold for each individual SNV in the panel |
| | Established by analysis of a large cohort of genome DNA to collect data on homozygous SNV genotypes |
| | Allows for differences in variance across SNVs within a panel |

### The Fixed Cutoff Method

In some embodiments, determining whether a polymorphic nucleic acid target is informative and/or detecting fetus-specific cell free nucleic acids comprises comparing its measured allele frequency in a mother to a fixed cutoff frequency. In some cases, determining which polymorphic nucleic acid targets are informative comprises identifying informative genotypes by comparing each allele frequency to one or more fixed cutoff frequencies. Fixed cutoff frequencies may be predetermined threshold values based on one or more qualifying data sets from a population of subjects who are not pregnant, for example, and represent the variance of the measured allele frequencies in subjects who are not pregnant.

In some cases, the fixed cutoff for identifying informative genotypes from non-informative genotypes is expressed as a percent (%) shift in allele frequency from an expected allele frequency. Generally, expected allele frequencies for a given allele (e.g., allele A) are 0 (for a BB genotype), 0.5 (for an AB genotype) and 1.0 (for an AA genotype), or equivalent values on any numerical scale. If a polymorphic nucleic acid target allele frequency in the mother deviate from an expected allele frequency and such deviation is beyond one or more fixed cutoff frequencies, the polymorphic nucleic acid target may be considered informative (i.e., the fetus has a different genotype from the mother). The degree of deviation generally is proportional to fetus-specific nucleic acid fraction (i.e., large deviations from expected allele frequency may be observed in samples having high fetus-specific nucleic acid fraction). The deviation between the expected allele frequency and measured allele frequency can be determined as described above.

In some cases, the polymorphic nucleic acid targets in the maternal genome before or during pregnancy are homozygous and the expected allele frequency, either the reference allele or the alternate allele, is, e.g., 0. In these circumstances, the deviation between the measured allele frequency in a sample from the pregnant mother and expected allele frequency is equal to the measured allele frequency. The polymorphic nucleic acid targets are identified as informative if the measured allele frequency is greater than the fixed cutoff.

In some cases, the fixed cutoff is a percentile value of the measure of allele frequencies of all the polymorphic nucleic acid targets used in the assay. In some embodiments, the percentile value is a 90, 95 or 98 percentile value.

In some cases, the fixed cutoff for identifying informative genotypes from non-informative homozygotes is about a 0.5% or greater shift in allele frequency from the median of expected allele frequencies. For example, a fixed cutoff may be about a 0.6%, 0.7%, 0.8%, 0.9%, 1%, 1.5%, 2%, 3%, 4%, 5%, 10% or greater shift in allele frequency. In some cases, the fixed cutoff for identifying informative genotypes from non-informative homozygotes is about a 1% or greater shift in allele frequency. In some cases, the fixed cutoff for identifying informative genotypes from non-informative homozygotes is about a 2% or greater shift in allele frequency. In some embodiments, the fixed cutoff for identifying informative genotypes from non-informative heterozygotes is about a 10% or greater shift in allele frequency. For example, a fixed cutoff may be about a 10%, 15%, 20%, 21%, 22%, 23%, 24%, 25%, 26%, 27%, 28%, 29%, 30%, 35%, 40%, 45%, 50%, 55%, 60%, 70%, 80% or greater shift in allele frequency. In some cases, the fixed cutoff for identifying informative genotypes from non-informative heterozygotes is about a 25% or greater shift in allele frequency. In some cases, the fixed cutoff for identifying informative genotypes from non-informative heterozygotes is about a 50% or greater shift in allele frequency.

### Target-Specific Threshold Method

In some embodiments, determining whether a polymorphic nucleic acid target is informative and/or detecting the fetus-specific allele comprises comparing its measured allele frequency to a target-specific threshold (e.g., a cutoff value). In some embodiments, target-specific threshold frequencies are determined for each polymorphic nucleic acid target. Typically, target-specific threshold frequency is determined based on the allele frequency variance for the corresponding polymorphic nucleic acid target. In some embodiments, variance of individual polymorphic targets can be represented by a median absolute deviation (MAD), for example. In some cases, determining a MAD value for each polymorphic nucleic acid target can generate unique (i.e., target-specific) threshold values. To determine median absolute deviation, measured allele frequency can be determined, for example, for multiple replicates (e.g., 5, 6, 7, 8, 9, 10, 15, 20 or more replicates) of a mother only nucleic acid sample (e.g., buffy coat sample). Each polymorphic target in each replicate will typically have a slightly different measured allele frequency due to PCR and/or sequencing errors, for example. A median allele frequency value can be identified for each polymorphic target. A deviation from the median for the remaining replicates can be calculated (i.e., the difference between the observed allele frequency and the median allele frequency). The absolute value of the deviations (i.e., negative values become positive) is taken and the median value of the absolute deviations is calculated to provide a median absolute deviation (MAD) for each polymorphic nucleic acid target. A target-specific threshold can be assigned, for example, as a multiple of the MAD (e.g., 1×MAD, 2×MAD, 3×MAD, 4×MAD or 5×MAD). Typically, polymorphic targets having less variance have a lower MAD and therefore a lower threshold value than more variable targets.

In some embodiments, the target-specific threshold is a percentile value of the measured allele frequencies of the polymorphic nucleic acid target used in the assay. In some embodiments, the percentile value is a 90, 95 or 98 percentile value.

### Dynamic Clustering Algorithm

In some embodiments, determining whether a polymorphic nucleic acid target is informative and/or detecting the fetus-specific allele comprises a dynamic clustering algorithm. Non-limiting examples of dynamic clustering algorithms include K-means, affinity propagation, mean-shift, spectral clustering, ward hierarchical clustering, agglomerative clustering, DBSCAN, Gaussian mixtures, and Birch. See, http://scikit-learn.org/stable/modules/clustering.html#k-means. Such algorithms may be implemented with a processor, a micro-processor, a computer system, in conjunction with memory and/or by a microprocessor controlled apparatus.

In some embodiments, the dynamic clustering algorithm is a k-means clustering. The k-means algorithm divides a set of samples into disjoint clusters, each described by the mean position of the samples in the cluster. The means are commonly referred to as cluster “centroids”. The k-means algorithm aims to choose centroids that minimize the inertia, or within-cluster sum of squares criterion. k-means is often referred to as Lloyd’s algorithm. In basic terms, the algorithm has three steps. The first step chooses the initial centroids, with the most basic method being to choose k samples from a dataset X. After initialization, k-means consists of looping between the two other steps. The first step assigns each sample to its nearest centroid. The second step creates new centroids by taking the mean value of all of the samples assigned to each previous centroid. The difference between the old and the new centroids are computed and the algorithm repeats these last two steps until this value is less than a threshold. In other words, it repeats until the centroids do not move significantly.

In some embodiments, the dynamic clustering comprises stratifying the one or more polymorphic nucleic acid targets in the cell-free nucleic acids into maternal homozygous group and maternal heterozygous group based on the measured allele frequency for a reference allele or an alternate allele for each of the polymorphic nucleic acid targets. Homozygous groups are clustered having a mean position of close to 0 or 1, and heterozygous group are clustered having a mean position of close to 0.5.

The method may further comprise stratifying maternal homozygous groups into non-informative and informative groups; and measuring the amounts of one or more polymorphic nucleic acid targets in the informative groups. In some embodiments, stratifying the maternal homozygous groups into non-informative and informative groups is based on whether the group contains fetus-specific alleles -informative groups are the groups that comprise distinct fetal alleles not derived from the mother that are not present in the maternal genome and non-informative groups comprise alleles from the fetus, indistinquishable from the maternal genome, where the informative SNVs are those within the cluster with higher mean or median allele frequency. These informative SNVs can be used to determine the fractional concentration of fetus-derived cfDNA.

In some embodiments, the k-means clustering process is repeated as described above to identify a cutoff for the informative SNVS. To find a cutoff, clustering is performed on SNVs with allele frequencies in the range of (0, 0.25). This results in 2 clusters where cluster 1 (the lower cluster) are non-informative SNVs (fetal and maternal alleles match) and cluster 2 (the higher cluster) are informative SNVs (fetus has at least one different allele than the mother). The cutoff is calculated as the average of the maximum of the first/lower cluster and the minimum of the second/upper cluster.

In some embodiments, to determine informative SNVs allele frequencies are first mirrored to generate mirrored allele frequencies. A mirrored allele frequency is the lesser value of the allele frequency of an allele and (1 – the allele frequency). This mirrors allele frequencies larger than 0.5 into a range of [0,0.5] and groups similar fetus-mother genotype combinations together (e.g. AAₘₒₜₕₑᵣ/AB_{fetus} with BBₘₒₜₕₑᵣ/AB_{fetus}). An “informative” SNV is identified as an SNV where the fetal genotype and the maternal genotype for the SNV are different. Defining the reference alleles as A and alternate alleles as B, there are 2 categories of informative SNVs:
1) Informative category 1 refers to the “Homo-Het” category, in which the mother is homozygous and the fetus is heterozygous (e.g. AAₘₒₜₕₑᵣ/AB_{fetus} or BBₘₒₜₕₑᵣ/AB_{fetus}).
2) Informative category 2 refers to the “Het-Homo” category, in which the mother is heterozygous and the fetus is homozygous (e.g. ABₘₒₜₕₑᵣ/AA_{fetus} or ABₘₒₜₕₑᵣ/BB_{fetus}).

In some embodiments, the informative SNVs selected for detecting fetus-specific nucleic acid and/or determining the fetus specific nucleic acid fraction do not include the category 2 SNVs. In some embodiments, the informative SNVs selected for detecting fetus-specific nucleic acid and/or determining the fetus specific nucleic acid fraction include both category 1 and category 2 SNVs. In some embodiments, the category 1 SNVs are used to detect fetus-specific nucleic acid and/or determining the fetus specific nucleic acid fraction first, and if the results is not conclusive, category 2 SNVs are then used to detect fetus-specific nucleic acid and/or determining the fetus specific nucleic acid fraction.

The non-informative SNVs can then be identified and removed by different approaches, e.g., a two-step clustering analysis. In some embodiments, the first step is an iteration of fuzzy K-means in the range of mirrored allele frequencies between 0 and 0.3 in order to determine a lower cutoff separating non-informative SNVs (e.g. AAₘₒₜₕₑᵣ/AA_{fetus}) from informative SNVs (e.g. AAₘₒₜₕₑᵣ/AB_{fetus}). In a second round of clustering, hard K-means clustering is performed between this lower cutoff and an allele frequency of 0.49 to determine the upper bound of the desired informative SNVs (e.g. separating AAₘₒₜₕₑᵣ/AB_{fetus} from ABₘₒₜₕₑᵣ/AA_{fetus} and ABₘₒₜₕₑᵣ/AB_{fetus}).

Two different approaches are detailed as follows, depending on availability of the genotype for the mother:
1) Approach 1 (Fetal Fraction 1 - “FF1”) :
   If mother’s genotype is not known, use K-means clustering to identify and remove non-informative SNVs (AAₘₒₜₕₑᵣ/AA_{fetus}, BBₘₒₜₕₑᵣ/BB_{fetus}, and ABₘₒₜₕₑᵣ/AB_{fetus}, ABₘₒₜₕₑᵣ/AA_{fetus}, and ABₘₒₜₕₑᵣ/BB_{fetus} Combinations). The 2 clusters are expected to contain the following mother/fetus genotype combinations:
      a. Cluster 1 = (AAₘₒₜₕₑᵣ/AB_{fetus}, BBₘₒₜₕₑᵣ/AB_{fetus},).
      b. Cluster 2 = (ABₘₒₜₕₑᵣ/AB_{fetus}, ABₘₒₜₕₑᵣ/AA_{fetus}, ABₘₒₜₕₑᵣ/BB_{fetus}). Retain only the SNVs in the cluster 1 as those are relevant to the fetus fraction calculation.

Accordingly, using the FF1 approach, under the circumstances where the mother’s genotype is not known, the method of determining paternity comprises:
I) Obtaining genotypes for the one or more SNVs in a genomic DNA sample obtained from an alleged father;
II) isolating cell-free nucleic acids from a biological sample obtained from the pregnant mother;
III) measuring the amount of each allele of the one or more SNVs in the biological sample to generate a data set consisting of measurements of the amounts of the one or more SNVs; an “informative” SNV is identified as an SNV where the fetus’s genotype and the mother’s genotype for the SNV are different.
IV) performing a computer algorithm on the data set to form a first cluster and a second cluster, wherein the first cluster comprising informative SNVs and the second cluster comprising non-informative SNVs,
   wherein the informative SNVs are present in the mother and the fetus in a genotype combination of AAₘₒₜₕₑᵣ/AB_{fetus}, BBₘₒₜₕₑᵣ/AB_{fetus}, , and
   wherein the non-informative SNVs are present in the mother and the fetus in a genotype combination of AAₘₒₜₕₑᵣ/AA_{fetus}, BBₘₒₜₕₑᵣ/BB_{fetus}, ABₘₒₜₕₑᵣ/AB_{fetus}, ABₘₒₜₕₑᵣ/AA_{fetus}, or ABₘₒₜₕₑᵣ/BB_{fetus};
V) detecting the fetus specific allele based on the presence of the informative SNVs. In some embodiments, the method further comprises determining the fetus-specific nucleic acid fraction based on the amount of the fetus specific alleles; and
VI) determining the paternity status of the fetus based on the genotypes of the mother, alleged father and the fetus for the informative nucleic acid targets.

2) Approach 2 (“FF2”):
   Approach 2 is used when the mother’s genotype is known.
      Approach 2A (“FF2A”)
   Approach 2A utilizes only SNVs where the mother is homozygous for paternity determination. In Approach 2A, the method comprises filtering out cases where the mother is heterozygous (so ABₘₒₜₕₑᵣ/AB_{fetus}, ABₘₒₜₕₑᵣ/AA_{fetus}, and ABₘₒₜₕₑᵣ/BB_{fetus} are excluded). Then perform clustering on the remaining SNVs to remove uninformative SNVs.The remaining informative SNVs have the following genotype combinations: AAₘₒₜₕₑᵣ/AB_{fetus}, BBₘₒₜₕₑᵣ/AB_{fetus}.
      SNVs in Cluster 1 are relevant to the fetus fraction calculation and should be retained.
   Accordingly, using the FF2A approach, under the circumstances where the mother’s genotype is known, the disclosure provides a method of paternity determination comprises:
      I) Obtaining genotypes for the one or more SNVs in a genomic DNA sample obtained from an alleged father;
      II) isolating cell-free nucleic acids from a biological sample obtained from the pregnant mother;
      III) measuring the amount of each allele of the one or more SNVs in the biological sample to generate a data set consisting of measurements of the amounts of the one or more SNVs;
      IV) filtering out SNVs which are present in the mother and the fetus in a genotype combination of ABₘₒₜₕₑᵣ/AB_{fetus}, ABₘₒₜₕₑᵣ/AA_{fetus}, and ABₘₒₜₕₑᵣ/BB_{fetus}, where
      V) the remaining SNVs are present in the mother and the fetus in a genotype combination of
         AAₘₒₜₕₑᵣ/BB_{fetus} or BBₘₒₜₕₑᵣ/AA_{fetus}, and AAₘₒₜₕₑᵣ/AB_{fetus} or BBₘₒₜₕₑᵣ/AB_{fetus} detecting the fetus specific allele based on the presence of the remaining SNVs in the one or more SNVs in the biological sample. In some embodiments, the method further comprises determining fetus-specific nucleic acid fraction in the biologoical sample based on the amount of the fetus specific alleles; and
      VI) determining the paternity status of the fetus based on the genotypes of the mother, alleged father and the fetus for the informative nucleic acid targets
         Approach 2B (“FF2B”):
         Approach 2B utilizes only SNVs where the mother’s genotype is heterozygous. Approach 2B comprises filtering out cases where the mother is homozygous (so AAₘₒₜₕₑᵣ/AB_{fetus}, BBₘₒₜₕₑᵣ/AB_{fetus}) are excluded. After removing the uninformative SNVs (AAₘₒₜₕₑᵣ/AA_{fetus}, BBₘₒₜₕₑᵣ/BB_{fetus}), the remaining SNVs are informative, which include genotype combinations of ABₘₒₜₕₑᵣ/AA_{fetus}, and ABₘₒₜₕₑᵣ/BB_{fetus}.. The amount of the fetus-specfic alleles can be determined, which can be used to determine the fetus genotype.

In some embodiments, the method of paternity determination may involve Approach 2A but not Approach 2B. In some embodiments, the method of paternity determination involves both Approach 2A and Approach 2B. In some embodiments, the method involves determining paternity using Approach 2A first, and if that determination is inconclusive, Approach 2B is used.

In some embodiments, Maximum Likelihood and Bayesian statistics (involving the application of the Bayes’ Theorem to experimental data) can be used to determine fetal genotype. Maximum likelihood is a statistical method that chooses the model that maximizes the probability of the observed data. Therefore, the probability of the observed data will be evaluated for each possible genotype, and the possible genotype that confers the highest probability on the observed data is chosen. Bayesian statistics are based on the likelihood of the data and prior probabilities of the hypoteses, which in this case would be the observed frequencies of the genotypes in the population (e.g., the expected allele frequency). Bayesian statistics provides the probability that a genotype is correct. For paternity determination, values of allele frequencies of the SNVs are analysed and hypotheses of possible genotypes of fetus and/or the mother are evaluated. The genotypes of the fetus are determined according to the hypothesis that has the highest likelihood based on the data (using the Maximum Likelihood), or that has a probability to be true, which is higher than a predetermined threshold (using bayesian statistics). In some embodiments, the SNVs used in the Maximum Likelihood and/or Bayesian statistics are informative SNVs that have been selected based on the other algorithms disclosed herein, for example, the clustering algorithm.

### Determining Paternity Status

### Calculating Fetus-Specific Cell-Free DNA Fraction (“Fetal Fraction”) and Fetal Genotypes

In some embodiments, the fetal fraction is calculated as the median of the frequencies across all informative SNVs. Informative SNVs are determined using any of the methods described above.

In some embodiments, a fraction or ratio can be determined for the amount of one nucleic acid relative to the amount of another nucleic acid. In some embodiments, the fraction of fetus-specific cell-free nucleic acid in a sample relative to the total amount of cell-free nucleic acid in the sample is determined. In general, to calculate the fraction of fetus-specific cell-free nucleic acid in a sample relative to the total amount of the cell-free nucleic acid in the sample, the following equation can be applied:
The fraction of fetus-specific cell-free nucleic acid = (amount of fetus-specific cell-free nucleic acid) / [(amount of total cell-free nucleic acid)].

In some embodiments, determining the fetus genotype starts with determining the allele frequencies of fetal-specific alleles for one or more informative polynucleic acid targets (e.g., informative SNVs), as described above. Even though it is not required for fetus genotyping or for paternity determination, determining fetal fraction is useful for quality control- if fetal fraction is not high enough, one may incorrectly estimate the paternity index and therefore mis-classify paternity. Lower fetal fractions tend to correspond to earlier gestation and also higher BMI of the mother. For reliable paternity determination, it is desirable that the fetal faction is at least 2%, at least 3%, at least 4%, at least 5%, or at least 10%. In some embodiments the fetal fraction in the cell-free samples ranges from 2% to 50%, from 4% to 40%, or from 6% to 30%.

In some embodiments, for a given SNV, fetal allele frequency is compared to a background frequency of the respective polymorphic nucleic acid target. That is to say, even if an allele is not actually present in the sample comprising fetal nucleic acids, a background proportion would still be detected due to, for example, sequencing errors. In some cases, the background frequency can be from about 0.001 to about 0.01 (i.e., 0.1% to about 1.0%). For example, background frequency can be about 0.002, 0.003, 0.004, 0.005, 0.006, 0.007, 0.008, or 0.009. In some cases, background frequency is about 0.005. Background frequencies for each allele of each SNVs can be determined empirically. For a given SNV, if fetal allele frequency is above background frequency, the genotype of the fetus can be confirmed to be different from that of the pregnant mother.

### Determining Paternity

Paternity can be determined by identifying informative SNVs and comparing fetal genotypes at the informative SNVs to the genotypes of one or more alleged fathers.

A paternity index can be determined for each informative SNV, which represents the likelihood that an alleged father is the biological father versus the likelihood that a random man, from the same population as the alleged father, is the biological father. The likelihood that a random man is the biological father is a function of the allele frequencies in the population, which are published.

In some embodiments, a combined paternity index (aka “likelihood ratio” or “LR”) is determined by multiplying the paternity index values for each informative SNV. The combined paternity index value can be used to determine paternity by comparing it with a threshold index. That is, a combined paternity index value above the threshold indicates that the alleged father is the biological father of the fetus. In some cases, a threshod for the combined paternity index value may range from about 2,000 to about 50,000. For example, the threshold can be at least 3,000, at least 4,000, at least 5,000, at least 10,000, at least 15,000, at least 20,000, at least 25,000, at least 30,000, or at least 40,000. In some cases, the paternity index threshold for determining paternity is about 10,000.

In some embodiments, the probability of paternity is calculated using Bayes’ Theorem. The probability of paternity is the posterior probability that the alleged father is the biological father and is calculated using the likelihoods and prior probabilities of the competing hypotheses. Methods for determining posterior probability are known and described in, e.g., Thore Egeland, Daniel Kling, and Petter Mostad. 2016. Relationship Inference with Familias and R, Statistical Methods in Forensic Genetics. Academic Press, Elsevier, e.g., pages 16-21 and pages 21-22. The entire content of said reference is herein incorporated by reference.

In some embodiments, maternal genotype, fetal genotype, and alleged father genotypes determined above can be analyzed using softwares that are known in the art, for example, Familas3 or extensions thereof (e.g., Famlink, FamlinkX, etc.) to determine the combined paternity index.

In some embodiments, other known software programs are used to perform paternity index calculations and/or paternity determination.

In some embodiments, the informative SNVs described above (i.e., those where the mother is homozygous and the fetus is heterozygous) are insufficient to determine paternity. That is, the calculated paternity index does not exceed the threshold value for determining paternity. In these cases, a second-round analysis can be performed to identify additional informative SNVs. In some embodiments, this second-round analysis involves identifying SNVs where the mother is heterozygous and the fetus is homozygous. For example, maximum likelihood analysis and Bayesian statistics can be applied to SNVs where the mother is heterozygous to determine whether the fetus is homozygous based on measured allele frequency. In some embodiments, SNVs for which the mother is heterozygous and the fetus is homozygous are also used to determining paternity, see the discussion of Approach 2A and Approach 2B, above.

### Quantification Of Polymorphic Nucleic Acid Targets

In some embodiments, the amount of the polymorphic nucleic acid targets are quantified based on sequence reads. In certain embodiments the quantity of sequence reads that are mapped to a polymorphic nucleic acid target on a reference genome for each allele is referred to as a count or read density. In certain embodiments, a count is determined from some or all of the sequence reads mapped to the polymorphic nucleic acid target.

A count can be determined by a suitable method, operation or mathematical process. A count sometimes is the direct sum of all sequence reads mapped to a genomic portion or a group of genomic portions corresponding to a segment, a group of portions corresponding to a sub-region of a genome (e.g., copy number variation region, copy number alteration region, copy number duplication region, copy number deletion region, microduplication region, microdeletion region, chromosome region, autosome region, sex chromosome region or other chromosomal rearrangement) and/or sometimes is a group of portions corresponding to a genome.

In some embodiments, a count is derived from raw sequence reads and/or filtered sequence reads. In certain embodiments a count is determined by a mathematical process. In certain embodiments a count is an average, mean or sum of sequence reads mapped to a target nucleic acid sequence on a reference genome for each of the two alleles (a reference allele and an alternate allele) of a polymorphic site. In some embodiments, a count is associated with an uncertainty value. A count sometimes is adjusted. A count may be adjusted according to sequence reads associated with a target nucleic acid sequence on a reference genome for each of the two alleles (a reference allele and an alternate allele) of a polymorphic site that have been weighted, removed, filtered, normalized, adjusted, averaged, derived as a mean, derived as a median, added, or combination thereof.

A sequence read quantification sometimes is a read density. A read density may be determined and/or generated for one or more segments of a genome. In certain instances, a read density may be determined and/or generated for one or more chromosomes. In some embodiments a read density comprises a quantitative measure of counts of sequence reads mapped to a a target nucleic acid sequence on a reference genome for each of the two alleles (a reference allele and an alternate allele) of a polymorphic site. A read density can be determined by a suitable process. In some embodiments a read density is determined by a suitable distribution and/or a suitable distribution function. Non-limiting examples of a distribution function include a probability function, probability distribution function, probability density function (PDF), a kernel density function (kernel density estimation), a cumulative distribution function, probability mass function, discrete probability distribution, an absolutely continuous univariate distribution, the like, any suitable distribution, or combinations thereof. A read density may be a density estimation derived from a suitable probability density function. A density estimation is the construction of an estimate, based on observed data, of an underlying probability density function. In some embodiments a read density comprises a density estimation (e.g., a probability density estimation, a kernel density estimation). A read density may be generated according to a process comprising generating a density estimation for each of the one or more portions of a genome where each portion comprises counts of sequence reads. A read density may be generated for normalized and/or weighted counts mapped to a portion or segment. In some instances, each read mapped to a portion or segment may contribute to a read density, a value (e.g., a count) equal to its weight obtained from a normalization process described herein. In some embodiments read densities for one or more portions or segments are adjusted. Read densities can be adjusted by a suitable method. For example, read densities for one or more portions can be weighted and/or normalized.

### Enriching Cell-Free Nucleic Acids

In some embodiments, the polymorphic nucleic acid targets are enriched before identifying the fetus-specific cell free nucleic acid using methods described herein. In some embodiments, enriching comprises amplifying the plurality of polymorphic nucleic acid targets. In some cases, the enriching comprises generating amplification products in an amplification reaction. Amplification of polymorphic targets may be achieved by any method described herein or known in the art for amplifying nucleic acid (e.g., PCR). In some cases, the amplification reaction is performed in a single vessel (e.g., tube, container, well on a plate) which sometimes is referred to herein as multiplexed amplification.

The amount of fetus-specific cell free nucleic acid can be quantified and used in conjunction with other methods for assessing paternity. The amount of fetus-specific nucleic acid can be determined in a nucleic acid sample from a subject before or after processing to prepare sample nucleic acid. In certain embodiments, the amount of fetus-specific nucleic acid is determined in a sample after sample nucleic acid is processed and prepared, which amount is utilized for further assessment. In some embodiments, an outcome comprises factoring the fraction of fetus-specific nucleic acid in the sample nucleic acid (e.g., adjusting counts, removing samples, making a call or not making a call).

In some embodiments, the cell-free nucleic acids from the sample derived from the pregnant mother can be enriched before determining the fetus-specific cell-free nucleic acids or quantifying the fetus-specific fraction. In some cases, the enrichment methods can include amplification (e.g., PCR)-based approaches.

### Amplification of Nucleotide Sequences

In many instances, it is desirable to amplify a nucleic acid sequence of the technology herein using any of several nucleic acid amplification procedures which are well known in the art (listed above and described in greater detail below). Specifically, nucleic acid amplification is the enzymatic synthesis of nucleic acid amplicons (copies) which contain a sequence that is complementary to a nucleic acid sequence being amplified. Nucleic acid amplification is especially beneficial when the amount of target sequence present in a sample is very low. By amplifying the target sequences and detecting the amplicon synthesized, the sensitivity of an assay can be vastly improved, since fewer target sequences are needed at the beginning of the assay to better ensure detection of nucleic acid in the sample belonging to the organism or virus of interest.

Any suitable amplification technique can be utilized. Amplification of polynucleotides include, but are not limited to, polymerase chain reaction (PCR); ligation amplification (or ligase chain reaction (LCR)); amplification methods based on the use of Q-beta replicase or template-dependent polymerase (see U.S. Pat. Publication No. US20050287592); helicase-dependant isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, digital PCR, combinations thereof, and the like. For example, amplification can be accomplished using digital PCR, in certain embodiments (see e.g. Kalinina et al., “Nanoliter scale PCR with TaqMan detection.” Nucleic Acids Research. 25; 1999-2004, (1997); Vogelstein and Kinzler (Digital PCR. Proc Natl Acad Sci USA. 96; 9236-41, (1999); PCT Patent Publication No. WO05023091A2; U.S. Pat. Publication No. US 20070202525). Digital PCR takes advantage of nucleic acid (DNA, cDNA or RNA) amplification on a single molecule level, and offers a highly sensitive method for quantifying low copy number nucleic acid. Systems for digital amplification and analysis of nucleic acids are available (e.g., Fluidigm® Corporation). Reagents and hardware for conducting PCR are commercially available.

In some embodiments, an amplification product may include naturally occurring nucleotides, non-naturally occurring nucleotides, nucleotide analogs and the like and combinations of the foregoing. An amplification product often has a nucleotide sequence that is identical to or substantially identical to a nucleic acid sequence herein, or complement thereof. A “substantially identical” nucleotide sequence in an amplification product will generally have a high degree of sequence identity to the nucleotide sequence species being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of infidelity of the polymerase used for extension and/or amplification, or additional nucleotide sequence(s) added to the primers used for amplification.

### Primers

Primers useful for detection, amplification, quantification, sequencing and analysis of nucleic acid are provided. The term “primer” as used herein refers to a nucleic acid that includes a nucleotide sequence capable of hybridizing or annealing to a target nucleic acid, at or near (e.g., adjacent to) a specific region of interest. Primers can allow for specific determination of a target nucleic acid nucleotide sequence or detection of the target nucleic acid (e.g., presence or absence of a sequence or copy number of a sequence), or feature thereof, for example. A primer may be naturally occurring or synthetic. The term “specific” or “specificity”, as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the term “anneal” refers to the formation of a stable complex between two molecules. The terms “primer”, “oligo”, or “oligonucleotide” may be used interchangeably throughout the document, when referring to primers.

A primer nucleic acid can be designed and synthesized using suitable processes, and may be of any length suitable for hybridizing to a nucleotide sequence of interest (e.g., where the nucleic acid is in liquid phase or bound to a solid support) and performing analysis processes described herein. Primers may be designed based upon a target nucleotide sequence. A primer in some embodiments may be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer may be composed of naturally occurring and/or non-naturally occurring nucleotides (e.g., labeled nucleotides), or a mixture thereof. Primers suitable for use with embodiments described herein, may be synthesized and labeled using known techniques. Primers may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of primers can be effected by native acrylamide gel electrophoresis or by anion-exchange high-performance liquid chromatography (HPLC), for example, as described in Pearson and Regnier, J. Chrom., 255: 137-149, 1983.

In some cases, loci-specific amplification methods can be used (e.g., using loci-specific amplification primers). In some cases, a multiplex SNV allele PCR approach can be used. In some cases, a multiplex SNV allele PCR approach can be used in combination with uniplex sequencing. For example, such an approach can involve the use of multiplex PCR (e.g., MASSARRAY system) and incorporation of capture probe sequences into the amplicons followed by sequencing using, for example, the Illumina MPSS system. In some cases, a multiplex SNV allele PCR approach can be used in combination with a three-primer system and indexed sequencing. For example, such an approach can involve the use of multiplex PCR (e.g., MASSARRAY system) with primers having a first capture probe incorporated into certain loci-specific forward PCR primers and adapter sequences incorporated into loci-specific reverse PCR primers, to thereby generate amplicons, followed by a secondary PCR to incorporate reverse capture sequences and molecular index barcodes for sequencing using, for example, the Illumina MPSS system. In some cases, a multiplex SNV allele PCR approach can be used in combination with a four-primer system and indexed sequencing. For example, such an approach can involve the use of multiplex PCR (e.g., MASSARRAY system) with primers having adaptor sequences incorporated into both loci-specific forward and loci-specific reverse PCR primers, followed by a secondary PCR to incorporate both forward and reverse capture sequences and molecular index barcodes for sequencing using, for example, the Illumina MPSS system. In some cases, a microfluidics approach can be used. In some cases, an array-based microfluidics approach can be used. For example, such an approach can involve the use of a microfluidics array (e.g., Fluidigm) for amplification at low plex and incorporation of index and capture probes, followed by sequencing. In some cases, an emulsion microfluidics approach can be used, such as, for example, digital droplet PCR.

In some cases, universal amplification methods can be used (e.g., using universal or non-loci-specific amplification primers). In some cases, universal amplification methods can be used in combination with pull-down approaches. In some cases, the method can include biotinylated ultramer pull-down (e.g., biotinylated pull-down assays from Agilent or IDT) from a universally amplified sequencing library. For example, such an approach can involve preparation of a standard library, enrichment for selected regions by a pull-down assay, and a secondary universal amplification step. In some cases, pull-down approaches can be used in combination with ligation-based methods. In some cases, the method can include biotinylated ultramer pull down with sequence specific adapter ligation (e.g., HALOPLEX PCR, Halo Genomics). For example, such an approach can involve the use of selector probes to capture restriction enzyme-digested fragments, followed by ligation of captured products to an adaptor, and universal amplification followed by sequencing. In some cases, pull-down approaches can be used in combination with extension and ligation-based methods. In some cases, the method can include molecular inversion probe (MIP) extension and ligation. For example, such an approach can involve the use of molecular inversion probes in combination with sequence adapters followed by universal amplification and sequencing. In some cases, complementary DNA can be synthesized and sequenced without amplification.

In some cases, extension and ligation approaches can be performed without a pull-down component. In some cases, the method can include loci-specific forward and reverse primer hybridization, extension and ligation. Such methods can further include universal amplification or complementary DNA synthesis without amplification, followed by sequencing. Such methods can reduce or exclude background sequences during analysis, in some cases.

In some cases, pull-down approaches can be used with an optional amplification component or with no amplification component. In some cases, the method can include a modified pull-down assay and ligation with full incorporation of capture probes without universal amplification. For example, such an approach can involve the use of modified selector probes to capture restriction enzyme-digested fragments, followed by ligation of captured products to an adaptor, optional amplification, and sequencing. In some cases, the method can include a biotinylated pull-down assay with extension and ligation of adaptor sequence in combination with circular single stranded ligation. For example, such an approach can involve the use of selector probes to capture regions of interest (i.e. target sequences), extension of the probes, adaptor ligation, single stranded circular ligation, optional amplification, and sequencing. In some cases, the analysis of the sequencing result can separate target sequences form background.

In some embodiments, nucleic acid is enriched for fragments from a select genomic region (e.g., chromosome) using one or more sequence-based separation methods described herein. Sequence-based separation generally is based on nucleotide sequences present in the fragments of interest (e.g., target and/or reference fragments) and substantially not present in other fragments of the sample or present in an insubstantial amount of the other fragments (e.g., 5% or less). In some embodiments, sequence-based separation can generate separated target fragments and/or separated reference fragments. Separated target fragments and/or separated reference fragments typically are isolated away from the remaining fragments in the nucleic acid sample. In some cases, the separated target fragments and the separated reference fragments also are isolated away from each other (e.g., isolated in separate assay compartments). In some cases, the separated target fragments and the separated reference fragments are isolated together (e.g., isolated in the same assay compartment). In some embodiments, unbound fragments can be differentially removed or degraded or digested.

In some embodiments, a selective nucleic acid capture process is used to separate target and/or reference fragments away from the nucleic acid sample. Commercially available nucleic acid capture systems include, for example, Nimblegen sequence capture system (Roche NimbleGen, Madison, WI); Illumina BEADARRAY platform (Illumina, San Diego, CA); Affymetrix GENECHIP platform (Affymetrix, Santa Clara, CA); Agilent SureSelect Target Enrichment System (Agilent Technologies, Santa Clara, CA); and related platforms. Such methods typically involve hybridization of a capture oligonucleotide to a portion or all of the nucleotide sequence of a target or reference fragment and can include use of a solid phase (e.g., solid phase array) and/or a solution based platform. Capture oligonucleotides (sometimes referred to as “bait”) can be selected or designed such that they preferentially hybridize to nucleic acid fragments from selected genomic regions or loci (e.g., one of chromosomes 21, 18, 13, X or Y, or a reference chromosome).

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is sometimes referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In some cases, length-based separation approaches can include fragment circularization, chemical treatment (e.g., formaldehyde, polyethylene glycol (PEG)), mass spectrometry and/or size-specific nucleic acid amplification, for example.

Certain length-based separation methods that can be used with methods described herein employ a selective sequence tagging approach, for example. In such methods, a fragment size species (e.g., short fragments) nucleic acids are selectively tagged in a sample that includes long and short nucleic acids. Such methods typically involve performing a nucleic acid amplification reaction using a set of nested primers which include inner primers and outer primers. In some cases, one or both of the inner can be tagged to thereby introduce a tag onto the target amplification product. The outer primers generally do not anneal to the short fragments that carry the (inner) target sequence. The inner primers can anneal to the short fragments and generate an amplification product that carries a tag and the target sequence. Typically, tagging of the long fragments is inhibited through a combination of mechanisms which include, for example, blocked extension of the inner primers by the prior annealing and extension of the outer primers. Enrichment for tagged fragments can be accomplished by any of a variety of methods, including for example, exonuclease digestion of single stranded nucleic acid and amplification of the tagged fragments using amplification primers specific for at least one tag.

Another length-based separation method that can be used with methods described herein involves subjecting a nucleic acid sample to polyethylene glycol (PEG) precipitation. Examples of methods include those described in International Patent Application Publication Nos. WO2007/140417 and WO2010/115016. This method in general entails contacting a nucleic acid sample with PEG in the presence of one or more monovalent salts under conditions sufficient to substantially precipitate large nucleic acids without substantially precipitating small (e.g., less than 300 nucleotides) nucleic acids.

Another size-based enrichment method that can be used with methods described herein involves circularization by ligation, for example, using circligase. Short nucleic acid fragments typically can be circularized with higher efficiency than long fragments. Non-circularized sequences can be separated from circularized sequences, and the enriched short fragments can be used for further analysis.

### Assays for Detecting the Polymorphic Nucleic Acid Targets

In some embodiments, the one or more polymorphic nucleic acid targets can be determined using one or more assays that are known in the art. Non-limiting examples of methods of detection, quantification, sequencing and the like include mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), direct DNA sequencing, Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization using at least one probe, hybridization using at least one fluorescently labeled probe, cloning and sequencing, electrophoresis, the use of hybridization probes and quantitative real time polymerase chain reaction (QRT-PCR), digital PCR, nanopore sequencing, chips and combinations thereof. In some embodiments the amount of each amplified nucleic acid species is determined by mass spectrometry, primer extension, sequencing (e.g., any suitable method, for example nanopore or pyrosequencing), Quantitative PCR (Q-PCR or QRT-PCR), digital PCR, combinations thereof, and the like.

In some embodiments, the assay is a sequencing reaction, as described herein. Sequencing, mapping and related analytical methods are known in the art (e.g., U.S. Pat. Application Publication US2009/0029377, incorporated by reference). Certain aspects of such processes are described hereafter.

In some embodiments, a polymorphic nucleic acid target can be detected using primers designed to amplify a region comprising the polymorphic nucleic acid target.

In some embodiments, a polymorphic nucleic acid target can be detected using a ligation-based assay using two probes flanking the polymorphic nucleic acid target, as further described below.

Any of the methods described above can be multiplexed by combining probes or primers that can be used to detect at least 5, at least 10, at least 100, or at least 200 polymorphic nucleic acid targets in one reaction. In some embodiments, the number of polymorphic nucleic acid targets that can be detected in the multiplexed reaction is in the range of between 20 and 10,000, e.g., between 30 and 5000, between 50 and 950, between 100 and 500, between 150 and 400, or between 200 and 350.

### Ligation Based Assays for Detecting SNV for Paternity Testing

### Probes

Probes useful for detection, quantification, sequencing and analysis of target nucleic acids are provided in embodiments described herein. In some embodiments, probes are used in sets, where a set contains a pair of probes. The term “probe”, as used herein refers to a nucleic acid that comprises a nucleotide sequence capable of hybridizing or annealing to a target nucleic acid, at or near (i.e., adjacent to) a specific region of interest.

In some embodiments, the polymorphic nucleic acid targets are the SNVs, for example, the SNVs disclosed in Table 1 or Table 5. Two probes, forming a probe pair, are designed to hybridize to the target region comprising each SNV under suitable conditions. One of the two probes is an allele-specific probe, i.e., it contains a nucleotide complementary to one specific allele of the SNV, and said nucleotide is at the end of the allele-specific probe that is proximal to the other probe in the probe pair (“partner probe”). The two probes are immediately adjacent to each other when hybridized to the target region. If the target region contains the specific allele, the two probes can be ligated by a DNA ligase and form a linked probe. If the target nucleic acid molecule does not contain the specific allele, the two probes will not ligate. The linked probe comprising the allele can be dissociated from the target (e.g., by denaturing) followed by sequencing to detect the specific allele.

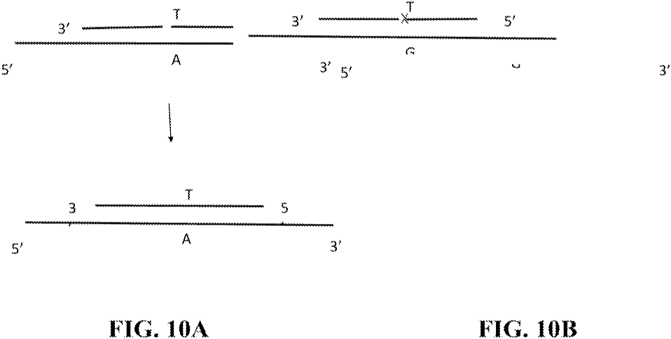

One illustrative example is shown in FIGS. 10A and 10B, where two probes form a probe pair, which are ligated to each other when both hybridized to the target comprising a specific allele at the SNV locus. Both probes include primer hybridization sequences that do not hybridize to the target nucleic acid molecule. The linked probe is then amplified and sequenced.

Probe pairs for detecting other alleles at the same SNV locus can be similarly designed. For example, a plurality of allele-specific probes (e.g., 2, 3, or 4 allele-specific probes), each comprising a nucleotide complementary to a different specific allele of the SNV at one end, can be used to detect all possible alleles at one SNV locus. Each allele-specific probe is paired with a partner probe to hybridize to the target region containing a specific allele of the SNV. The allele-specific probe and its partner probe are immediately adjacent to each other. The linked probes formed from the ligation of these probe pairs are sequenced to detect the various alleles of the SNV.

In one illustrative embodiment, two DNA probes are designed to detect each allele genotype of each SNV in Table 5. For example, if there are two alleles, A and G, at an SNV locus, two probes are designed to detect the A allele, and two probes are designed to detect the G allele.

In some embodiments, one or both probes comprise one or more additional sequences, for example, one or more sequences for identifying sample origin (i.e., a unique sample identifier), one or more primer binding sequences for hybridizing to amplification primers, and/or one or more primber binding sequences for hybridizing sequencing primers. In some embodiments, the amplification primers are universal primers. After the dissociation of the linked probe from the target nucleic acid molecule, amplification primers are annealed to the linked probe to create copies of the linked probe.

In some embodiments, the linked probes are amplified before sequencing. The linked probes (or the amplified linked probes) can be sequenced, and sequence reads for the linked probes comprising various alleles for the SNV can be counted. The allele frequency for each allele at this SNV locus can be determined based on the number of sequence reads for all different alleles for the SNV. Informative SNVs are selected based on the allele frequencies as described above, which, combined with the information of the genotype of the pregnant mother and the alleged father, can be used to determine whether the alleged father is the biological father using methods disclosed herein, for example, the above sections entitled “selecting polymorphic nucleic acid targets,” “identifying the informative polymorphic nucleic acid targets,” and “Determining paternity status.”

In some embodiments, the relative abundance of fetus-specific cell-free nucleic acid in a recipient sample can be determined as a parameter of the total number of unique sequence reads mapped to a target nucleic acid sequence on a reference genome for each of the alleles (a reference allele and one or more alternate alleles) of a polymorphic site. In some embodiments, the assay is a high throughput sequencing. In some embodiments, the assay is a digital polymerase chain reaction (dPCR). In some embodiments, the assay is a microarray analysis.

In some embodiments, the sequencing process is a sequencing by synthesis method, as described herein. Typically, sequencing by synthesis methods comprise a plurality of synthesis cycles, whereby a complementary nucleotide is added to a single stranded template and identified during each cycle. The number of cycles generally corresponds to read length. In some cases, polymorphic targets are selected such that a minimal read length (i.e., minimal number of cycles) is required to include amplification primer sequence and the polymorphic target site (e.g., SNV) in the read. In some cases, amplification primer sequence includes about 10 to about 30 nucleotides. For example, amplification primer sequence may include about 11, 12, 13, 14, 15, 16, 17, 18, 19, 20, 21, 22, 23, 24, 25, 26, 27, 28, or 29 nucleotides, in some embodiments. In some cases, amplification primer sequence includes about 20 nucleotides. In some embodiments, a SNV site is located within 1 nucleotide base position (i.e., adjacent to) to about 30 base positions from the 3′ terminus of an amplification primer. For example, a SNV site may be within 2, 3, 4, 5, 6, 7, 8, 9, 10, 11, 12, 13, 14, 15, 16, 17, 18, 19, 20, 21, 22, 23, 24, 25, 26, 27, 28, or 29 nucleotides of an amplification primer terminus. Read lengths can be any length that is inclusive of an amplification primer sequence and a polymorphic sequence or position. In some embodiments, read lengths can be about 10 nucleotides in length to about 50 nucleotides in length. For example, read lengths can be about 15, 20, 21, 22, 23, 24, 25, 26, 27, 28, 29, 30, 31, 32, 33, 34, 35, 36, 37, 38, 39, 40, or 45 nucleotides in length. In some cases, read length is about 36 nucleotides. In some cases, read length is about 27 nucleotides. Thus, in some cases, the sequencing by synthesis method comprises about 36 cycles and sometimes comprises about 27 cycles.

In some embodiments, a plurality of samples is sequenced in a single compartment (e.g., flow cell), which sometimes is referred to herein as sample multiplexing. Thus, in some embodiments, fetus-specific nucleic acid fraction is determined for a plurality of samples in a multiplexed assay. For example, fetus-specific nucleic acid fraction may be determined for about 10, 20, 30, 40 , 50, 60, 70, 80, 90, 100, 200, 300, 400, 500, 600, 700, 800, 900, 1000, 2000 or more samples. In some cases, fetus-specific nucleic acid fraction is determined for about 10 or more samples. In some cases, fetus-specific nucleic acid fraction is determined for about 100 or more samples. In some cases, fetus-specific nucleic acid fraction is determined for about 1000 or more samples.

Typically, sequence reads are monitored and filtered to exclude low quality sequence reads. The term “filtering” as used herein refers to removing a portion of data or a set of data from consideration and retaining a subset of data. Sequence reads can be selected for removal based on any suitable criteria, including but not limited to redundant data (e.g., redundant or overlapping mapped reads), non-informative data, over represented or underrepresented sequences, noisy data, the like, or combinations of the foregoing. A filtering process often involves removing one or more reads and/or read pairs (e.g., discordant read pairs) from consideration. Reducing the number of reads, pairs of reads and/or reads comprising candidate SNVs from a data set analyzed for the presence or absence of an informative SNV often reduces the complexity and/or dimensionality of a data set, and sometimes increases the speed of searching for and/or identifying informative SNVs by two or more orders of magnitude.

Nucleic acid detection and/or quantification also may include, for example, solid support array based detection of fluorescently labeled nucleic acid with fluorescent labels incorporated during or after PCR, single molecule detection of fluorescently labeled molecules in solution or captured on a solid phase, or other sequencing technologies such as, for example, sequencing using ION TORRENT or MISEQ platforms or single molecule sequencing technologies using instrumentation such as, for example, PACBIO sequencers, HELICOS sequencer, or nanopore sequencing technologies.

In some cases, nucleic acid quantifications generated by a method comprising a sequencing detection process may be compared to nucleic acid quantifications generated by a method comprising a different detection process (e.g., mass spectrometry). Such comparisons may be expressed using an R² value, which is a measure of correlation between two outcomes (e.g., nucleic acid quantifications). In some cases, nucleic acid quantifications (e.g., fetal copy number quantifications) are highly correlated (i.e., have high R² values) for quantifications generated using different detection processes (e.g., sequencing and mass spectrometry). In some cases, R² values for nucleic acid quantifications generated using different detection processes may be between about 0.90 and about 1.0. For example, R² values may be about 0.91, 0.92, 0.93, 0.94, 0.95, 0.96, 0.97, 0.98, or 0.99.

In some embodiments, the polymorphic nucleic acid targets are restriction fragment length polymorphisms (RFLPs). RFLPs detection may be performed by cleaving the nucleic acid with an enzyme and evaluated with a probe that hybridize to the cleaved products and thus defines a uniquely sized restriction fragment corresponding to an allele. RFLPs can be used to detect fetal cell-free nucleic acids. As an illustrative example, where a homozygous mother would have only a single fragment generated by a particular restriction enzyme which hybridizes to a restriction fragment length polymorphism probe, during pregnancy with a heterozygous fetus, the cell-free nucleic acids in the pregnant mother would have two distinctly sized fragments which hybridize to the same probe generated by the enzyme. Therefore detecting the RFLPs can be used to identify the presence of the fetus-specific cell-free nucleic acids.

Techniques for polynucleotide sequence determination are also well established and widely practiced in the relevant research field. For instance, the basic principles and general techniques for polynucleotide sequencing are described in various research reports and treatises on molecular biology and recombinant genetics, such as Wallace et al., supra; Sambrook and Russell, supra, and Ausubel et al., supra. DNA sequencing methods routinely practiced in research laboratories, either manual or automated, can be used for practicing the present technology. Additional means suitable for detecting changes in a polynucleotide sequence for practicing the methods of the present technology include but are not limited to mass spectrometry, primer extension, polynucleotide hybridization, real-time PCR, and electrophoresis.

Use of a primer extension reaction also can be applied in methods of the technology herein. A primer extension reaction operates, for example, by discriminating the SNV alleles by the incorporation of deoxynucleotides and/or dideoxynucleotides to a primer extension primer which hybridizes to a region adjacent to the SNV site. The primer is extended with a polymerase. The primer extended SNV can be detected physically by mass spectrometry or by a tagging moiety such as biotin. As the SNV site is only extended by a complementary deoxynucleotide or dideoxynucleotide that is either tagged by a specific label or generates a primer extension product with a specific mass, the SNV alleles can be discriminated and quantified.

Reverse transcribed and amplified nucleic acids may be modified nucleic acids. Modified nucleic acids can include nucleotide analogs, and in certain embodiments include a detectable label and/or a capture agent. Examples of detectable labels include without limitation fluorophores, radioisotopes, colormetric agents, light emitting agents, chemiluminescent agents, light scattering agents, enzymes and the like. Examples of capture agents include without limitation an agent from a binding pair selected from antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B 12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) pairs, and the like. Modified nucleic acids having a capture agent can be immobilized to a solid support in certain embodiments

Mass spectrometry is a particularly effective method for the detection of a polynucleotide of the technology herein, for example a PCR amplicon, a primer extension product or a detector probe that is cleaved from a target nucleic acid. The presence of the polynucleotide sequence is verified by comparing the mass of the detected signal with the expected mass of the polynucleotide of interest. The relative signal strength, e.g., mass peak on a spectra, for a particular polynucleotide sequence indicates the relative population of a specific allele, thus enabling calculation of the allele ratio directly from the data. For a review of genotyping methods using Sequenom® standard iPLEX™ assay and MassARRAYⓇ technology, see Jurinke, C., Oeth, P., van den Boom, D., “MALDI-TOF mass spectrometry: a versatile tool for high-performance DNA analysis.” Mol. Biotechnol. 26, 147-164 (2004); and Oeth, P. et al., “iPLEX™ Assay: Increased Plexing Efficiency and Flexibility for MassARRAY® System through single base primer extension with mass-modified Terminators.” SEQUENOM Application Note (2005), both of which are hereby incorporated by reference. For a review of detecting and quantifying target nucleic acids using cleavable detector probes that are cleaved during the amplification process and detected by mass spectrometry, see U.S. Pat. Application No. 11/950,395, which was filed Dec. 4, 2007, and is hereby incorporated by reference.

Various sequencing techniques that are suitable for use include, but not limited to sequencing-by-synthesis, reversible terminator-based sequencing, 454 sequencing (Roche) (Margulies, M. et al. 2005 Nature 437, 376-380), Applied Biosystems’ SOLiD™ technology, Helicos True Single Molecule Sequencing (tSMS), single molecule, real-time (SMRT™) sequencing technology of Pacific Biosciences, ION TORRENT (Life Technologies) single molecule sequencing, chemical-sensitive field effect transistor (CHEMFET) array, electron microscopy sequencing technology, digital PCR, sequencing by hybridization, nanopore sequencing, Illumina Genome Analyzer (or Solexa platform) or SOLiD System (Applied Biosystems) or the Helicos True Single Molecule DNA sequencing technology (Harris T D et al. 2008 Science, 320, 106-109), the single molecule, real-time (SMRT.™.) technology of Pacific Biosciences, and nanopore sequencing (Soni GV and Meller A. 2007 Clin Chem 53: 1996-2001). Many of these methods allow the sequencing of many nucleic acid molecules isolated from a specimen at high orders of multiplexing in a parallel fashion (Dear Brief Funct Genomic Proteomic 2003; 1: 397-416).

Many sequencing platforms that allow sequencing of clonally expanded or non-amplified single molecules of nucleic acid fragments can be used for detecting the fetus-specific cell-free nucleic acids. Certain platforms involve, for example, (i) sequencing by ligation of dye-modified probes (including cyclic ligation and cleavage), (ii) pyrosequencing, and (iii) single-molecule sequencing. Nucleotide sequence species, amplification nucleic acid species and detectable products generated there from can be considered a “study nucleic acid” for purposes of analyzing a nucleotide sequence by such sequence analysis platforms.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label (e.g., 1 fluorescent label, 2, 3, or 4 fluorescent labels).

An example of a system that can be used by a person of ordinary skill based on sequencing by ligation generally involves the following steps. Clonal bead populations can be prepared in emulsion microreactors containing study nucleic acid (“template”), amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag. Such a system can be used to exponentially amplify amplification products generated by a process described herein, e.g., by ligating a heterologous nucleic acid to the first amplification product generated by a process described herein and performing emulsion amplification using the same or a different solid support originally used to generate the first amplification product. Such a system also may be used to analyze amplification products directly generated by a process described herein by bypassing an exponential amplification process and directly sorting the solid supports described herein on the glass slide.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination.

An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)). Such a system can be used to exponentially amplify amplification products generated by a process described herein, e.g., by ligating a heterologous nucleic acid to the first amplification product generated by a process described herein.

Certain single-molecule sequencing embodiments are based on the principal of sequencing by synthesis, and utilize single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair”, in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a study nucleic acid to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslavsky et al., PNAS 100(7): 3960-3964 (2003)). Such a system can be used to directly sequence amplification products generated by processes described herein. In some embodiments the released linear amplification product can be hybridized to a primer that contains sequences complementary to immobilized capture sequences present on a solid support, a bead or glass slide for example. Hybridization of the primer--released linear amplification product complexes with the immobilized capture sequences, immobilizes released linear amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer--released linear amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting sample nucleic acid and solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support. Single nucleotide sequencing methods useful in the embodiments described herein are described in U.S. Provisional Pat. Application Serial No. 61/021,871 filed Jan. 17, 2008.

In certain embodiments, nanopore sequencing detection methods include (a) contacting a nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known to the person of ordinary skill (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons sometimes are performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis is facilitated by sequence analysis apparatus and components known to the person of ordinary skill in the art.

Methods provided herein allow for high-throughput detection of nucleic acid species in a plurality of nucleic acids (e.g., nucleotide sequence species, amplified nucleic acid species and detectable products generated from the foregoing). Multiplexing refers to the simultaneous detection of more than one nucleic acid species. General methods for performing multiplexed reactions in conjunction with mass spectrometry, are known (see, e.g., U.S. Pat. Nos. 6,043,031, 5,547,835 and International PCT Application No. WO 97/37041). Multiplexing provides an advantage that a plurality of nucleic acid species (e.g., some having different sequence variations) can be identified in as few as a single mass spectrum, as compared to having to perform a separate mass spectrometry analysis for each individual target nucleic acid species. Methods provided herein lend themselves to high-throughput, highly-automated processes for analyzing sequence variations with high speed and accuracy, in some embodiments. In some embodiments, methods herein may be multiplexed at high levels in a single reaction.

In certain embodiments, the number of nucleic acid species multiplexed include, without limitation, about 1 to about 500 (e.g., about 1-3, 3-5, 5-7, 7-9, 9-11, 11-13, 13-15, 15-17, 17-19, 19-21, 21-23, 23-25, 25-27, 27-29, 29-31, 31-33, 33-35, 35-37, 37-39, 39-41, 41-43, 43-45, 45-47, 47-49, 49-51, 51-53, 53-55, 55-57, 57-59, 59-61, 61-63, 63-65, 65-67, 67-69, 69-71, 71-73, 73-75, 75-77, 77-79, 79-81, 81-83, 83-85, 85-87, 87-89, 89-91, 91-93, 93-95, 95-97, 97-101, 101-103, 103-105, 105-107, 107-109, 109-111, 111-113, 113-115, 115-117, 117-119, 121-123, 123-125, 125-127, 127-129, 129-131, 131-133, 133-135, 135-137, 137-139, 139-141, 141-143, 143-145, 145-147, 147-149, 149-151, 151-153, 153-155, 155-157, 157-159, 159-161, 161-163, 163-165, 165-167, 167-169, 169-171, 171-173, 173-175, 175-177, 177-179, 179-181, 181-183, 183-185, 185-187, 187-189, 189-191, 191-193, 193-195, 195-197, 197-199, 199-201, 201-203, 203-205, 205-207, 207-209, 209-211, 211-213, 213-215, 215-217, 217-219, 219-221, 221-223, 223-225, 225-227, 227-229, 229-231, 231-233, 233-235, 235-237, 237-239, 239-241, 241-243, 243-245, 245-247, 247-249, 249-251, 251-253, 253-255, 255-257, 257-259, 259-261, 261-263, 263-265, 265-267, 267-269, 269-271, 271-273, 273-275, 275-277, 277-279, 279-281, 281-283, 283-285, 285-287, 287-289, 289-291, 291-293, 293-295, 295-297, 297-299, 299-301, 301- 303, 303- 305, 305- 307, 307- 309, 309- 311, 311-313, 313- 315, 315- 317, 317- 319, 319-321, 321-323, 323-325, 325-327, 327-329, 329-331, 331-333, 333- 335, 335-337, 337-339, 339-341, 341-343, 343-345, 345-347, 347-349, 349-351, 351-353, 353-355, 355-357, 357-359, 359-361, 361-363, 363-365, 365-367, 367-369, 369-371, 371-373, 373-375, 375-377, 377-379, 379-381, 381-383, 383-385, 385-387, 387-389, 389-391, 391-393, 393-395, 395-397, 397-401, 401- 403, 403- 405, 405- 407, 407- 409, 409- 411, 411- 413, 413- 415, 415- 417, 417- 419, 419-421, 421-423, 423-425, 425-427, 427-429, 429-431, 431-433, 433- 435, 435-437, 437-439, 439-441, 441-443, 443-445, 445-447, 447-449, 449-451, 451-453, 453-455, 455-457, 457-459, 459-461, 461-463, 463-465, 465-467, 467-469, 469-471, 471-473, 473-475, 475-477, 477-479, 479-481, 481-483, 483-485, 485-487, 487-489, 489-491, 491-493, 493-495, 495-497, 497-501).

Design methods for achieving resolved mass spectra with multiplexed assays can include primer and oligonucleotide design methods and reaction design methods. For primer and oligonucleotide design in multiplexed assays, the same general guidelines for primer design applies for uniplexed reactions, such as avoiding false priming and primer dimers, only more primers are involved for multiplex reactions. For mass spectrometry applications, analyte peaks in the mass spectra for one assay are sufficiently resolved from a product of any assay with which that assay is multiplexed, including pausing peaks and any other by-product peaks. Also, analyte peaks optimally fall within a user-specified mass window, for example, within a range of 5,000-8,500 Da. In some embodiments multiplex analysis may be adapted to mass spectrometric detection of chromosome abnormalities, for example. In certain embodiments multiplex analysis may be adapted to various single nucleotide or nanopore based sequencing methods described herein. Commercially produced micro-reaction chambers or devices or arrays or chips may be used to facilitate multiplex analysis, and are commercially available.

### Adaptors

In some embodiments, nucleic acids (e.g., PCR primers, PCR amplicons, sample nucleic acid) may include an adaptor sequence and/or complement thereof. Adaptor sequences often are useful for certain sequencing methods such as, for example, a sequencing-by-synthesis process described herein. Adaptors sometimes are referred to as sequencing adaptors or adaptor oligonucleotides. Adaptor sequences typically include one or more sites useful for attachment to a solid support (e.g., flow cell). Adaptors also may include sequencing primer hybridization sites (i.e. sequences complementary to primers used in a sequencing reaction) and identifiers (e.g., indices) as described below. Adaptor sequences can be located at the 5′ and/or 3′ end of a nucleic acid and sometimes can be located within a larger nucleic acid sequence. Adaptors can be any length and any sequence, and may be selected based on standard methods in the art for adaptor design.

One or more adaptor oligonucleotides may be incorporated into a nucleic acid (e.g., PCR amplicon) by any method suitable for incorporating adaptor sequences into a nucleic acid. For example, PCR primers used for generating PCR amplicons (i.e., amplification products) may comprise adaptor sequences or complements thereof. Thus, PCR amplicons that comprise one or more adaptor sequences can be generated during an amplification process. In some cases, one or more adaptor sequences can be ligated to a nucleic acid (e.g., PCR amplicon) by any ligation method suitable for attaching adaptor sequences to a nucleic acid. Ligation processes may include, for example, blunt-end ligations, ligations that exploit 3′ adenine (A) overhangs generated by Taq polymerase during an amplification process and ligate adaptors having 3′ thymine (T) overhangs, and other “sticky-end” ligations. Ligation processes can be optimized such that adaptor sequences hybridize to each end of a nucleic acid and not to each other.

In some cases, adaptor ligation is bidirectional, which means that adaptor sequences are attached to a nucleic acid such that both ends of the nucleic acid are sequenced in a subsequent sequencing process. In some cases, adaptor ligation is unidirectional, which means that adaptor sequences are attached to a nucleic acid such that one end of the nucleic acid is sequenced in a subsequent sequencing process. Examples of unidirectional and bidirectional ligation schemes are as described in US20170058350, the entire disclosure is hereby incorporated by reference.

### Identifiers

In some embodiments, nucleic acids (e.g., PCR primers, PCR amplicons, sample nucleic acid, sequencing adaptors) may include an identifier. In some cases, an identifier is located within or adjacent to an adaptor sequence. An identifier can be any feature that can identify a particular origin or aspect of a nucleic acid target sequence. For example, an identifier (e.g., a sample identifier) can identify the sample from which a particular nucleic acid target sequence originated. In another example, an identifier (e.g., a sample aliquot identifier) can identify the sample aliquot from which a particular nucleic acid target sequence originated. In another example, an identifier (e.g., chromosome identifier) can identify the chromosome from which a particular nucleic acid target sequence originated. An identifier may be referred to herein as a tag, index, barcode, identification tag, index primer, and the like. An identifier may be a unique sequence of nucleotides (e.g., sequence-based identifiers), a detectable label such as the labels described below (e.g., identifier labels), and/or a particular length of polynucleotide (e.g., length-based identifiers; size-based identifiers) such as a stuffer sequence. Identifiers for a collection of samples or plurality of chromosomes, for example, may each comprise a unique sequence of nucleotides. Identifiers (e.g., sequence-based identifiers, length-based identifiers) may be of any length suitable to distinguish certain target genomic sequences from other target genomic sequences. In some embodiments, identifiers may be from about one to about 100 nucleotides in length. For example, identifiers independently may be about 1, 2, 3, 4, 5, 6, 7, 8, 9, 10, 20, 30, 40, 50, 60, 70, 80, 90 or 100 nucleotides in length. In some embodiments, an identifier contains a sequence of six nucleotides. In some cases, an identifier is part of an adaptor sequence for a sequencing process, such as, for example, a sequencing-by-synthesis process described in further detail herein. In some cases, an identifier may be a repeated sequence of a single nucleotide (e.g., poly-A, poly-T, poly-G, poly-C). Such identifiers may be detected and distinguished from each other, for example, using nanopore technology, as described herein.

In some embodiments, the analysis includes analyzing (e.g., detecting, counting, processing counts for, and the like) the identifier. In some embodiments, the detection process includes detecting the identifier and sometimes not detecting other features (e.g., sequences) of a nucleic acid. In some embodiments, the counting process includes counting each identifier. In some embodiments, the identifier is the only feature of a nucleic acid that is detected, analyzed and/or counted.

### Sequencing

Any sequencing method suitable for conducting methods described herein can be utilized. In some embodiments, a high-throughput sequencing method is used. High-throughput sequencing methods generally involve clonally amplified DNA templates or single DNA molecules that are sequenced in a massively parallel fashion within a flow cell (e.g. as described in Metzker M Nature Rev 11:31-46 (2010); Volkerding et al. Clin Chem 55:641-658 (2009)). Such sequencing methods also can provide digital quantitative information, where each sequence read is a countable “sequence tag” or “count” representing an individual clonal DNA template or a single DNA molecule. High-throughput sequencing technologies include, for example, sequencing-by-synthesis with reversible dye terminators, sequencing by oligonucleotide probe ligation, pyrosequencing and real time sequencing.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

In some embodiments, first generation technology, such as, for example, Sanger sequencing including the automated Sanger sequencing, can be used in the methods provided herein. Additional sequencing technologies that include the use of developing nucleic acid imaging technologies (e.g. transmission electron microscopy (TEM) and atomic force microscopy (AFM)), also are contemplated herein. Examples of various sequencing technologies are described below.

The length of the sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, the sequence reads are of a mean, median or average length of about 15 bp to 900 bp long (e.g. about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp or more.

In some embodiments, nucleic acids may include a fluorescent signal or sequence tag information. Quantification of the signal or tag may be used in a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

### Data Processing And Normalization

In some embodiments, sequence read data that are used to represent the amount of a polymorphic nucleic acid target can be processed further (e.g., mathematically and/or statistically manipulated) and/or displayed to facilitate providing an outcome. In certain embodiments, data sets, including larger data sets, may benefit from pre-processing to facilitate further analysis. Pre-processing of data sets sometimes involves removal of redundant and/or uninformative portions or portions of a reference genome (e.g., portions of a reference genome with uninformative data, redundant mapped reads, portions with zero median counts, over represented or underrepresented sequences). Without being limited by theory, data processing and/or preprocessing may (i) remove noisy data, (ii) remove uninformative data, (iii) remove redundant data, (iv) reduce the complexity of larger data sets, and/or (v) facilitate transformation of the data from one form into one or more other forms. The terms “pre-processing” and “processing” when utilized with respect to data or data sets are collectively referred to herein as “processing.” Processing can render data more amenable to further analysis, and can generate an outcome in some embodiments. In some embodiments one or more or all processing methods (e.g., normalization methods, portion filtering, mapping, validation, the like or combinations thereof) are performed by a processor, a microprocessor, a computer, in conjunction with memory and/or by a microprocessor controlled apparatus.

The term “noisy data” as used herein refers to (a) data that has a significant variance between data points when analyzed or plotted, (b) data that has a significant standard deviation (e.g., greater than 3 standard deviations), (c) data that has a significant standard error of the mean, the like, and combinations of the foregoing. Noisy data sometimes occurs due to the quantity and/or quality of starting material (e.g., nucleic acid sample), and sometimes occurs as part of processes for preparing or replicating DNA used to generate sequence reads. In certain embodiments, noise results from certain sequences being overrepresented when prepared using PCR-based methods. Methods described herein can reduce or eliminate the contribution of noisy data, and therefore reduce the effect of noisy data on the provided outcome.

The terms “uninformative data,” “uninformative portions of a reference genome,” and “uninformative portions” as used herein refer to portions, or data derived therefrom, having a numerical value that is significantly different from a predetermined threshold value or falls outside a predetermined cutoff range of values. The terms “threshold” and “threshold value” herein refer to any number that is calculated using a qualifying data set and serves as a limit of diagnosis of a genetic variation or genetic alteration (e.g., a copy number alteration, an aneuploidy, a microduplication, a microdeletion, a chromosomal aberration, and the like). In certain embodiments, a threshold is exceeded by results obtained by methods described herein and a subject is diagnosed with a copy number alteration. A threshold value or range of values often is calculated by mathematically and/or statistically manipulating sequence read data (e.g., from a reference and/or subject), in some embodiments, and in certain embodiments, sequence read data manipulated to generate a threshold value or range of values is sequence read data (e.g., from a reference and/or subject). In some embodiments, an uncertainty value is determined. An uncertainty value generally is a measure of variance or error and can be any suitable measure of variance or error. In some embodiments an uncertainty value is a standard deviation, standard error, calculated variance, p-value, or mean absolute deviation (MAD). In some embodiments an uncertainty value can be calculated according to a formula described herein.

Any suitable procedure can be utilized for processing data sets described herein. Non-limiting examples of procedures suitable for use for processing data sets include filtering, normalizing, weighting, monitoring peak heights, monitoring peak areas, monitoring peak edges, peak level analysis, peak width analysis, peak edge location analysis, peak lateral tolerances, determining area ratios, mathematical processing of data, statistical processing of data, application of statistical algorithms, analysis with fixed variables, analysis with optimized variables, plotting data to identify patterns or trends for additional processing, the like and combinations of the foregoing. In some embodiments, data sets are processed based on various features (e.g., GC content, redundant mapped reads, centromere regions, telomere regions, the like and combinations thereof) and/or variables (e.g., subject gender, subject age, subject ploidy, percent contribution of cancer cell nucleic acid, fetal gender, maternal age, maternal ploidy, percent contribution of fetal nucleic acid, the like or combinations thereof). In certain embodiments, processing data sets as described herein can reduce the complexity and/or dimensionality of large and/or complex data sets. A non-limiting example of a complex data set includes sequence read data generated from one or more test subjects (e.g., pregnant mothers) and a plurality of reference subjects of different ages and ethnic backgrounds. In some embodiments, data sets can include from thousands to millions of sequence reads for each test and/or reference subject.

Data processing can be performed in any number of steps, in certain embodiments. For example, data may be processed using only a single processing procedure in some embodiments, and in certain embodiments data may be processed using 1 or more, 5 or more, 10 or more or 20 or more processing steps (e.g., 1 or more processing steps, 2 or more processing steps, 3 or more processing steps, 4 or more processing steps, 5 or more processing steps, 6 or more processing steps, 7 or more processing steps, 8 or more processing steps, 9 or more processing steps, 10 or more processing steps, 11 or more processing steps, 12 or more processing steps, 13 or more processing steps, 14 or more processing steps, 15 or more processing steps, 16 or more processing steps, 17 or more processing steps, 18 or more processing steps, 19 or more processing steps, or 20 or more processing steps). In some embodiments, processing steps may be the same step repeated two or more times (e.g., filtering two or more times, normalizing two or more times), and in certain embodiments, processing steps may be two or more different processing steps (e.g., filtering, normalizing; normalizing, monitoring peak heights and edges; filtering, normalizing, normalizing to a reference, statistical manipulation to determine p-values, and the like), carried out simultaneously or sequentially. In some embodiments, any suitable number and/or combination of the same or different processing steps can be utilized to process sequence read data to facilitate providing an outcome. In certain embodiments, processing data sets by the criteria described herein may reduce the complexity and/or dimensionality of a data set.

In some embodiments one or more processing steps can comprise one or more normalization steps. Normalization can be performed by a suitable method described herein or known in the art. In certain embodiments, normalization comprises adjusting values measured on different scales to a notionally common scale. In certain embodiments, normalization comprises a sophisticated mathematical adjustment to bring probability distributions of adjusted values into alignment. In some embodiments normalization comprises aligning distributions to a normal distribution. In certain embodiments normalization comprises mathematical adjustments that allow comparison of corresponding normalized values for different datasets in a way that eliminates the effects of certain gross influences (e.g., error and anomalies). In certain embodiments normalization comprises scaling. Normalization sometimes comprises division of one or more data sets by a predetermined variable or formula. Normalization sometimes comprises subtraction of one or more data sets by a predetermined variable or formula. Non-limiting examples of normalization methods include portion-wise normalization, normalization by GC content, median count (median bin count, median portion count) normalization, linear and nonlinear least squares regression, LOESS, GC LOESS, LOWESS (locally weighted scatterplot smoothing), principal component normalization, repeat masking (RM), GC-normalization and repeat masking (GCRM), cQn and/or combinations thereof. In some embodiments, the determination of a presence or absence of a copy number alteration (e.g., an aneuploidy, a microduplication, a microdeletion) utilizes a normalization method (e.g., portion-wise normalization, normalization by GC content, median count (median bin count, median portion count) normalization, linear and nonlinear least squares regression, LOESS, GC LOESS, LOWESS (locally weighted scatterplot smoothing), principal component normalization, repeat masking (RM), GC-normalization and repeat masking (GCRM), cQn, a normalization method known in the art and/or a combination thereof). Described in greater detail hereafter are certain examples of normalization processes that can be utilized, such as LOESS normalization, principal component normalization, and hybrid normalization methods, for example. Aspects of certain normalization processes also are described, for example, in International Patent Application Publication No. WO2013/052913 and International Patent Application Publication No. WO2015/051163, each of which is incorporated by reference herein.

Any suitable number of normalizations can be used. In some embodiments, data sets can be normalized 1 or more, 5 or more, 10 or more or even 20 or more times. Data sets can be normalized to values (e.g., normalizing value) representative of any suitable feature or variable (e.g., sample data, reference data, or both). Non-limiting examples of types of data normalizations that can be used include normalizing raw count data for one or more selected test or reference portions to the total number of counts mapped to the chromosome or the entire genome on which the selected portion or sections are mapped; normalizing raw count data for one or more selected portions to a median reference count for one or more portions or the chromosome on which a selected portion is mapped; normalizing raw count data to previously normalized data or derivatives thereof; and normalizing previously normalized data to one or more other predetermined normalization variables. Normalizing a data set sometimes has the effect of isolating statistical error, depending on the feature or property selected as the predetermined normalization variable. Normalizing a data set sometimes also allows comparison of data characteristics of data having different scales, by bringing the data to a common scale (e.g., predetermined normalization variable). In some embodiments, one or more normalizations to a statistically derived value can be utilized to minimize data differences and diminish the importance of outlying data. Normalizing portions, or portions of a reference genome, with respect to a normalizing value sometimes is referred to as “portion-wise normalization.”

In certain embodiments, a processing step can comprise one or more mathematical and/or statistical manipulations. Any suitable mathematical and/or statistical manipulation, alone or in combination, may be used to analyze and/or manipulate a data set described herein. Any suitable number of mathematical and/or statistical manipulations can be used. In some embodiments, a data set can be mathematically and/or statistically manipulated 1 or more, 5 or more, 10 or more or 20 or more times. Non-limiting examples of mathematical and statistical manipulations that can be used include addition, subtraction, multiplication, division, algebraic functions, least squares estimators, curve fitting, differential equations, rational polynomials, double polynomials, orthogonal polynomials, z-scores, p-values, chi values, phi values, analysis of peak levels, determination of peak edge locations, calculation of peak area ratios, analysis of median chromosomal level, calculation of mean absolute deviation, sum of squared residuals, mean, standard deviation, standard error, the like or combinations thereof. A mathematical and/or statistical manipulation can be performed on all or a portion of sequence read data, or processed products thereof. Non-limiting examples of data set variables or features that can be statistically manipulated include raw counts, filtered counts, normalized counts, peak heights, peak widths, peak areas, peak edges, lateral tolerances, P-values, median levels, mean levels, count distribution within a genomic region, relative representation of nucleic acid species, the like or combinations thereof.

In some embodiments, a processing step can comprise the use of one or more statistical algorithms. Any suitable statistical algorithm, alone or in combination, may be used to analyze and/or manipulate a data set described herein. Any suitable number of statistical algorithms can be used. In some embodiments, a data set can be analyzed using 1 or more, 5 or more, 10 or more or 20 or more statistical algorithms. Non-limiting examples of statistical algorithms suitable for use with methods described herein include principal component analysis, decision trees, counternulls, multiple comparisons, omnibus test, Behrens-Fisher problem, bootstrapping, Fisher’s method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing. Non-limiting examples of data set variables or features that can be analyzed using statistical algorithms include raw counts, filtered counts, normalized counts, peak heights, peak widths, peak edges, lateral tolerances, P-values, median levels, mean levels, count distribution within a genomic region, relative representation of nucleic acid species, the like or combinations thereof.

In certain embodiments, a data set can be analyzed by utilizing multiple (e.g., 2 or more) statistical algorithms (e.g., least squares regression, principal component analysis, linear discriminant analysis, quadratic discriminant analysis, bagging, neural networks, support vector machine models, random forests, classification tree models, K-nearest neighbors, logistic regression and/or smoothing) and/or mathematical and/or statistical manipulations (e.g., referred to herein as manipulations). The use of multiple manipulations can generate an N-dimensional space that can be used to provide an outcome, in some embodiments. In certain embodiments, analysis of a data set by utilizing multiple manipulations can reduce the complexity and/or dimensionality of the data set. For example, the use of multiple manipulations on a reference data set can generate an N-dimensional space (e.g., probability plot) that can be used to represent the presence or absence of a genetic variation/genetic alteration and/or copy number alteration, depending on the status of the reference samples (e.g., positive or negative for a selected copy number alteration). Analysis of test samples using a substantially similar set of manipulations can be used to generate an N-dimensional point for each of the test samples. The complexity and/or dimensionality of a test subject data set sometimes is reduced to a single value or N-dimensional point that can be readily compared to the N-dimensional space generated from the reference data. Test sample data that fall within the N-dimensional space populated by the reference subject data are indicative of a genetic status substantially similar to that of the reference subjects. Test sample data that fall outside of the N-dimensional space populated by the reference subject data are indicative of a genetic status substantially dissimilar to that of the reference subjects. In some embodiments, references are euploid or do not otherwise have a genetic variation/genetic alteration and/or copy number alteration and/or medical condition.

After data sets have been counted, optionally filtered, normalized, and optionally weighted the processed data sets can be further manipulated by one or more filtering and/or normalizing and/or weighting procedures, in some embodiments. A data set that has been further manipulated by one or more filtering and/or normalizing and/or weighting procedures can be used to generate a profile, in certain embodiments. The one or more filtering and/or normalizing and/or weighting procedures sometimes can reduce data set complexity and/or dimensionality, in some embodiments. An outcome can be provided based on a data set of reduced complexity and/or dimensionality. In some embodiments, a profile plot of processed data further manipulated by weighting, for example, is generated to facilitate classification and/or providing an outcome. An outcome can be provided based on a profile plot of weighted data, for example.

Filtering or weighting of portions can be performed at one or more suitable points in an analysis. For example, portions may be filtered or weighted before or after sequence reads are mapped to portions of a reference genome. Portions may be filtered or weighted before or after an experimental bias for individual genome portions is determined in some embodiments. In certain embodiments, portions may be filtered or weighted before or after levels are calculated.

After data sets have been counted, optionally filtered, normalized, and optionally weighted, the processed data sets can be manipulated by one or more mathematical and/or statistical (e.g., statistical functions or statistical algorithm) manipulations, in some embodiments. In certain embodiments, processed data sets can be further manipulated by calculating Z-scores for one or more selected portions, chromosomes, or portions of chromosomes. In some embodiments, processed data sets can be further manipulated by calculating P-values. In certain embodiments, mathematical and/or statistical manipulations include one or more assumptions pertaining to ploidy and/or fraction of a minority species (e.g., fraction of cancer cell nucleic acid; fetal fraction). In some embodiments, a profile plot of processed data further manipulated by one or more statistical and/or mathematical manipulations is generated to facilitate classification and/or providing an outcome. An outcome can be provided based on a profile plot of statistically and/or mathematically manipulated data. An outcome provided based on a profile plot of statistically and/or mathematically manipulated data often includes one or more assumptions pertaining to ploidy and/or fraction of a minority species (e.g., fraction of cancer cell nucleic acid; fetal fraction).

In some embodiments, analysis and processing of data can include the use of one or more assumptions. A suitable number or type of assumptions can be utilized to analyze or process a data set. Non-limiting examples of assumptions that can be used for data processing and/or analysis include subject ploidy, cancer cell contribution, maternal ploidy, fetal contribution, prevalence of certain sequences in a reference population, ethnic background, prevalence of a selected medical condition in related family members, parallelism between raw count profiles from different patients and/or runs after GC-normalization and repeat masking (e.g., GCRM), identical matches represent PCR artifacts (e.g., identical base position), assumptions inherent in a nucleic acid quantification assay (e.g., fetal quantifier assay (FQA)), assumptions regarding twins (e.g., if 2 twins and only 1 is affected the effective fetal fraction is only 50% of the total measured fetal fraction (similarly for triplets, quadruplets and the like)), cell free DNA (e.g., cfDNA) uniformly covers the entire genome, the like and combinations thereof.

In those instances where the quality and/or depth of mapped sequence reads does not permit an outcome prediction of the presence or absence of a genetic variation/genetic alteration and/or copy number alteration at a desired confidence level (e.g., 95% or higher confidence level), based on the normalized count profiles, one or more additional mathematical manipulation algorithms and/or statistical prediction algorithms, can be utilized to generate additional numerical values useful for data analysis and/or providing an outcome. The term “normalized count profile” as used herein refers to a profile generated using normalized counts. Examples of methods that can be used to generate normalized counts and normalized count profiles are described herein. As noted, mapped sequence reads that have been counted can be normalized with respect to test sample counts or reference sample counts. In some embodiments, a normalized count profile can be presented as a plot.

Described in greater detail hereafter are non-limiting examples of processing steps and normalization methods that can be utilized, such as normalizing to a window (static or sliding), weighting, determining bias relationship, LOESS normalization, principal component normalization, hybrid normalization, generating a profile and performing a comparison.

### Normalizing to a Window (Static or Sliding)

In certain embodiments, a processing step comprises normalizing to a static window, and in some embodiments, a processing step comprises normalizing to a moving or sliding window. The term “window” as used herein refers to one or more portions chosen for analysis, and sometimes is used as a reference for comparison (e.g., used for normalization and/or other mathematical or statistical manipulation). The term “normalizing to a static window” as used herein refers to a normalization process using one or more portions selected for comparison between a test subject and reference subject data set. In some embodiments the selected portions are utilized to generate a profile. A static window generally includes a predetermined set of portions that do not change during manipulations and/or analysis. The terms “normalizing to a moving window” and “normalizing to a sliding window” as used herein refer to normalizations performed to portions localized to the genomic region (e.g., immediate surrounding portions, adjacent portion or sections, and the like) of a selected test portion, where one or more selected test portions are normalized to portions immediately surrounding the selected test portion. In certain embodiments, the selected portions are utilized to generate a profile. A sliding or moving window normalization often includes repeatedly moving or sliding to an adjacent test portion, and normalizing the newly selected test portion to portions immediately surrounding or adjacent to the newly selected test portion, where adjacent windows have one or more portions in common. In certain embodiments, a plurality of selected test portions and/or chromosomes can be analyzed by a sliding window process.

In some embodiments, normalizing to a sliding or moving window can generate one or more values, where each value represents normalization to a different set of reference portions selected from different regions of a genome (e.g., chromosome). In certain embodiments, the one or more values generated are cumulative sums (e.g., a numerical estimate of the integral of the normalized count profile over the selected portion, domain (e.g., part of chromosome), or chromosome). The values generated by the sliding or moving window process can be used to generate a profile and facilitate arriving at an outcome. In some embodiments, cumulative sums of one or more portions can be displayed as a function of genomic position. Moving or sliding window analysis sometimes is used to analyze a genome for the presence or absence of microdeletions and/or microduplications. In certain embodiments, displaying cumulative sums of one or more portions is used to identify the presence or absence of regions of copy number alteration (e.g., microdeletion, microduplication).

### Weighting

In some embodiments, a processing step comprises a weighting. The terms “weighted,” “weighting” or “weight function” or grammatical derivatives or equivalents thereof, as used herein, refer to a mathematical manipulation of a portion or all of a data set sometimes utilized to alter the influence of certain data set features or variables with respect to other data set features or variables (e.g., increase or decrease the significance and/or contribution of data contained in one or more portions or portions of a reference genome, based on the quality or usefulness of the data in the selected portion or portions of a reference genome). A weighting function can be used to increase the influence of data with a relatively small measurement variance, and/or to decrease the influence of data with a relatively large measurement variance, in some embodiments. For example, portions of a reference genome with underrepresented or low quality sequence data can be “down weighted” to minimize the influence on a data set, whereas selected portions of a reference genome can be “up weighted” to increase the influence on a data set. A non-limiting example of a weighting function is [1 / (standard deviation)²]. Weighting portions sometimes removes portion dependencies. In some embodiments one or more portions are weighted by an eigen function (e.g., an eigenfunction). In some embodiments an eigen function comprises replacing portions with orthogonal eigen-portions. A weighting step sometimes is performed in a manner substantially similar to a normalizing step. In some embodiments, a data set is adjusted (e.g., divided, multiplied, added, subtracted) by a predetermined variable (e.g., weighting variable). In some embodiments, a data set is divided by a predetermined variable (e.g., weighting variable). A predetermined variable (e.g., minimized target function, Phi) often is selected to weigh different parts of a data set differently (e.g., increase the influence of certain data types while decreasing the influence of other data types).

### Bias Relationships

In some embodiments, a processing step comprises determining a bias relationship. For example, one or more relationships may be generated between local genome bias estimates and bias frequencies. The term “relationship” as use herein refers to a mathematical and/or a graphical relationship between two or more variables or values. A relationship can be generated by a suitable mathematical and/or graphical process. Non-limiting examples of a relationship include a mathematical and/or graphical representation of a function, a correlation, a distribution, a linear or non-linear equation, a line, a regression, a fitted regression, the like or a combination thereof. Sometimes a relationship comprises a fitted relationship. In some embodiments a fitted relationship comprises a fitted regression. Sometimes a relationship comprises two or more variables or values that are weighted. In some embodiments a relationship comprise a fitted regression where one or more variables or values of the relationship a weighted. Sometimes a regression is fitted in a weighted fashion. Sometimes a regression is fitted without weighting. In certain embodiments, generating a relationship comprises plotting or graphing.

In certain embodiments, a relationship is generated between GC densities and GC density frequencies. In some embodiments generating a relationship between (i) GC densities and (ii) GC density frequencies for a sample provides a sample GC density relationship. In some embodiments generating a relationship between (i) GC densities and (ii) GC density frequencies for a reference provides a reference GC density relationship. In some embodiments, where local genome bias estimates are GC densities, a sample bias relationship is a sample GC density relationship and a reference bias relationship is a reference GC density relationship. GC densities of a reference GC density relationship and/or a sample GC density relationship are often representations (e.g., mathematical or quantitative representation) of local GC content.

In some embodiments a relationship between local genome bias estimates and bias frequencies comprises a distribution. In some embodiments a relationship between local genome bias estimates and bias frequencies comprises a fitted relationship (e.g., a fitted regression). In some embodiments a relationship between local genome bias estimates and bias frequencies comprises a fitted linear or non-linear regression (e.g., a polynomial regression). In certain embodiments a relationship between local genome bias estimates and bias frequencies comprises a weighted relationship where local genome bias estimates and/or bias frequencies are weighted by a suitable process. In some embodiments a weighted fitted relationship (e.g., a weighted fitting) can be obtained by a process comprising a quantile regression, parameterized distributions or an empirical distribution with interpolation. In certain embodiments a relationship between local genome bias estimates and bias frequencies for a test sample, a reference or part thereof, comprises a polynomial regression where local genome bias estimates are weighted. In some embodiments a weighed fitted model comprises weighting values of a distribution. Values of a distribution can be weighted by a suitable process. In some embodiments, values located near tails of a distribution are provided less weight than values closer to the median of the distribution. For example, for a distribution between local genome bias estimates (e.g., GC densities) and bias frequencies (e.g., GC density frequencies), a weight is determined according to the bias frequency for a given local genome bias estimate, where local genome bias estimates comprising bias frequencies closer to the mean of a distribution are provided greater weight than local genome bias estimates comprising bias frequencies further from the mean.

In some embodiments, a processing step comprises normalizing sequence read counts by comparing local genome bias estimates of sequence reads of a test sample to local genome bias estimates of a reference (e.g., a reference genome, or part thereof). In some embodiments, counts of sequence reads are normalized by comparing bias frequencies of local genome bias estimates of a test sample to bias frequencies of local genome bias estimates of a reference. In some embodiments counts of sequence reads are normalized by comparing a sample bias relationship and a reference bias relationship, thereby generating a comparison.

Counts of sequence reads may be normalized according to a comparison of two or more relationships. In certain embodiments two or more relationships are compared thereby providing a comparison that is used for reducing local bias in sequence reads (e.g., normalizing counts). Two or more relationships can be compared by a suitable method. In some embodiments a comparison comprises adding, subtracting, multiplying and/or dividing a first relationship from a second relationship. In certain embodiments comparing two or more relationships comprises a use of a suitable linear regression and/or a non-linear regression. In certain embodiments comparing two or more relationships comprises a suitable polynomial regression (e.g., a 3^{rd} order polynomial regression). In some embodiments a comparison comprises adding, subtracting, multiplying and/or dividing a first regression from a second regression. In some embodiments two or more relationships are compared by a process comprising an inferential framework of multiple regressions. In some embodiments two or more relationships are compared by a process comprising a suitable multivariate analysis. In some embodiments two or more relationships are compared by a process comprising a basis function (e.g., a blending function, e.g., polynomial bases, Fourier bases, or the like), splines, a radial basis function and/or wavelets.

In certain embodiments a distribution of local genome bias estimates comprising bias frequencies for a test sample and a reference is compared by a process comprising a polynomial regression where local genome bias estimates are weighted. In some embodiments a polynomial regression is generated between (i) ratios, each of which ratios comprises bias frequencies of local genome bias estimates of a reference and bias frequencies of local genome bias estimates of a sample and (ii) local genome bias estimates. In some embodiments a polynomial regression is generated between (i) a ratio of bias frequencies of local genome bias estimates of a reference to bias frequencies of local genome bias estimates of a sample and (ii) local genome bias estimates. In some embodiments a comparison of a distribution of local genome bias estimates for reads of a test sample and a reference comprises determining a log ratio (e.g., a log2 ratio) of bias frequencies of local genome bias estimates for the reference and the sample. In some embodiments a comparison of a distribution of local genome bias estimates comprises dividing a log ratio (e.g., a log2 ratio) of bias frequencies of local genome bias estimates for the reference by a log ratio (e.g., a log2 ratio) of bias frequencies of local genome bias estimates for the sample.

Normalizing counts according to a comparison typically adjusts some counts and not others. Normalizing counts sometimes adjusts all counts and sometimes does not adjust any counts of sequence reads. A count for a sequence read sometimes is normalized by a process that comprises determining a weighting factor and sometimes the process does not include directly generating and utilizing a weighting factor. Normalizing counts according to a comparison sometimes comprises determining a weighting factor for each count of a sequence read. A weighting factor is often specific to a sequence read and is applied to a count of a specific sequence read. A weighting factor is often determined according to a comparison of two or more bias relationships (e.g., a sample bias relationship compared to a reference bias relationship). A normalized count is often determined by adjusting a count value according to a weighting factor.

Adjusting a count according to a weighting factor sometimes includes adding, subtracting, multiplying and/or dividing a count for a sequence read by a weighting factor. A weighting factor and/or a normalized count sometimes are determined from a regression (e.g., a regression line). A normalized count is sometimes obtained directly from a regression line (e.g., a fitted regression line) resulting from a comparison between bias frequencies of local genome bias estimates of a reference (e.g., a reference genome) and a test sample. In some embodiments each count of a read of a sample is provided a normalized count value according to a comparison of (i) bias frequencies of a local genome bias estimates of reads compared to (ii) bias frequencies of a local genome bias estimates of a reference. In certain embodiments, counts of sequence reads obtained for a sample are normalized and bias in the sequence reads is reduced.

### Machines, Sytems, Software and Interfaces

Certain processes and methods described herein (e.g., obtaining and filtering sequencing reads, determining if a polymorphic nucleic acid target is informative, or determining if one or more cell-free nucleic acid is a fetus-specific nucleic acid, using the fixed cutoff, dynamic k-means clustering, or individual polymorphic nucleic acid target threshold) often cannot be performed without a computer, microprocessor, software, module or other machine. Methods described herein typically are computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein is a computer system configured to perform the any of the embodiments of the methods for determining paternity disclosed herein. In some embodiments, this disclosure provides a system for determining paternity comprising one or more processors and non-transitory machine readable storage medium and/or memory coupled to one or more processors, and the memory or the non-transitory machine readable storage medium encoded with a set of instructions configured to perform a process comprising: (a) obtaining measurements of one or more polymorphic nucleic acid targets within the circulating cell-free nucleic acids isolated from a biological sample, wherein the biological sample is obtained from a pregnant mother; (b) detecting, by a computing system, one or more fetus-specific circulating cell-free nucleic acids based on the measurements from (a); and (c) determining paternity based on the presence or amount of said one or more fetus-specific nucleic acids.

In some embodiments, the set of instructions further comprise instructions for determining whether a polymorphic nucleic acid target is informative, and/or detecting fetus-specific cell-free nucleic acids in a sample from a test subject’s sample according to, for example, one of more of the fixed cutoff approach, a dynamic clustering approach, and/or an individual polymorphic nucleic acid target threshold approach as described above. In some cases, the instructions to reduce experimental bias is according to a GC normalized quantification of sequence reads.

Also provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein. In some embodiments, the program module instructs the microprocessor to perform a process comprising:(a) obtaining measurements of one or more polymorphic nucleic acid targets within the circulating cell-free nucleic acids isolated from a biological sample, wherein the biological sample is obtained from a pregnant mother; (b) detecting, by a computing system, one or more fetus-specific circulating cell-free nucleic acids based on the measurements from (a); and (c) determining paternity based on the presence or amount of said one or more fetus-specific nucleic acids The executable program stored on the computer reasable storage media may further instruct the microprocessor to determine whether a polymorphic nucleic acid target is informative, and/or detect fetus-specific cell-free nucleic acids in a sample from a test subject (a pregnant mother)’s sample according to, for example, one of more of the fixed cutoff approach, a dynamic clustering approach, and/or an individual polymorphic nucleic acid target threshold approach as described above.

In some embodiments, the disclosure provides a non-transitory machine readable storage medium comprising program instructions that when executed by one or more processors cause the one or more processors to perform a method, the method comprising: (a) obtaining measurements of one or more polymorphic nucleic acid targets within the circulating cell-free nucleic acids isolated from a biological sample, wherein the biological sample is obtained from a pregnant mother; (b) detecting, by a computing system, one or more fetus-specific circulating cell-free nucleic acids based on the measurements from (a); and (c) determining paternity based on the presence or amount of said one or more fetus-specific nucleic acids The program instructions may further comprise instructions for the one or more processors to determine whether a polymorphic nucleic acid target is informative, and/or detect fetus-specific cell-free nucleic acids in a sample from a pregnant mother, according to, for example, one of more of the fixed cutoff approach, a dynamic clustering approach, and/or an individual polymorphic nucleic acid target threshold approach as described above.

The non-transitory machine readable storage medium may further comprise program instructions that when executed by one or more processors cause the one or more processors to perform a method comprising: adjusting the quantified sequence reads for each of the genomic portions by an adjustment process that reduces experimental bias, wherein the adjustment process generates a normalized quantification of sequence reads for each of the polymorphic nucleic acid targets.

Thus, also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., (e.g., obtaining and filtering sequencing reads, determining if a polymorphic nucleic acid target is an informative, or determining if one or more cell-free nucleic acid is a fetus-specific nucleic acid, using the fixed cutoff, dynamic k-means clustering, or individual polymorphic nucleic acid target threshold) are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Sequence reads, counts, levels and/or measurements sometimes are referred to as “data” or “data sets.” In some embodiments, data or data sets can be characterized by one or more features or variables (e.g., sequence based (e.g., GC content, specific nucleotide sequence, the like), function specific (e.g., expressed genes, cancer genes, the like), location based (genome specific, chromosome specific, portion or portion-specific), the like and combinations thereof). In certain embodiments, data or data sets can be organized into a matrix having two or more dimensions based on one or more features or variables. Data organized into matrices can be organized using any suitable features or variables. In certain embodiments, data sets characterized by one or more features or variables sometimes are processed after counting.

Machines, software and interfaces may be used to conduct methods described herein. Using machines, software and interfaces, a user may enter, request, query or determine options for using particular information, programs or processes (e.g., mapping sequence reads, processing mapped data and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine a classification outcome from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein.

A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to an machine, peripheral, component or another module. A module can perform one or more of the following non-limiting functions: mapping sequence reads, providing counts, assembling portions, providing or determining a level, providing a count profile, normalizing (e.g., normalizing reads, normalizing counts, and the like), providing a normalized count profile or levels of normalized counts, comparing two or more levels, providing uncertainty values, providing or determining expected levels and expected ranges(e.g., expected level ranges, threshold ranges and threshold levels), providing adjustments to levels (e.g., adjusting a first level, adjusting a second level, adjusting a profile of a chromosome or a part thereof, and/or padding), providing identification (e.g., identifying a copy number alteration, genetic variation/genetic alteration or aneuploidy), categorizing, plotting, and/or determining an outcome, for example. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. Sequence read quantifications (e.g., counts) sometimes are accessed by a microprocessor that executes instructions configured to carry out a method described herein. Sequence read quantifications that are accessed by a microprocessor can be within memory of a system, and the counts can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

Software comprising program instructions often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using, for example, algorithms described herein to determine fetus-specific nuclic acids such as a fixed cutoff algorithm, a dynamic clustering algorithm, or an individual polymorphic nucleic acid target threshold algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining the presence or absence of a genetic variation or genetic alteration.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

FIG. 2 illustrates a non-limiting example of a computing environment 110 in which various systems, methods, algorithms, and data structures described herein may be implemented. The computing environment 110 is only one example of a suitable computing environment and is not intended to suggest any limitation as to the scope of use or functionality of the systems, methods, and data structures described herein. Neither should computing environment 110 be interpreted as having any dependency or requirement relating to any one or combination of components illustrated in computing environment 110. A subset of systems, methods, and data structures shown in FIG. 2 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The operating environment 110 of FIG. 2 includes a general purpose computing device in the form of a computer 120, including a processing unit 121, a system memory 122, and a system bus 123 that operatively couples various system components including the system memory 122 to the processing unit 121. There may be only one or there may be more than one processing unit 121, such that the processor of computer 120 includes a single central-processing unit (CPU), or a plurality of processing units, commonly referred to as a parallel processing environment. The computer 120 may be a conventional computer, a distributed computer, or any other type of computer.

The system bus 123 may be any of several types of bus structures including a memory bus or memory controller, a peripheral bus, and a local bus using any of a variety of bus architectures. The system memory may also be referred to as simply the memory, and includes read only memory (ROM) 124 and random access memory (RAM). A basic input/output system (BIOS) 126, containing the basic routines that help to transfer information between elements within the computer 120, such as during start-up, is stored in ROM 124. The computer 120 may further include a hard disk drive interface 127 for reading from and writing to a hard disk, not shown, a magnetic disk drive 128 for reading from or writing to a removable magnetic disk 129, and an optical disk drive 130 for reading from or writing to a removable optical disk 131 such as a CD ROM or other optical media.

The hard disk drive 127, magnetic disk drive 128, and optical disk drive 130 are connected to the system bus 123 by a hard disk drive interface 132, a magnetic disk drive interface 133, and an optical disk drive interface 134, respectively. The drives and their associated computer-readable media provide nonvolatile storage of computer-readable instructions, data structures, program modules and other data for the computer 120. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk 129, optical disk 131, ROM 124, or RAM, including an operating system 135, one or more application programs 136, other program modules 137, and program data 138. A user may enter commands and information into the personal computer 120 through input devices such as a keyboard 140 and pointing device 142. Other input devices (not shown) may include a microphone, joystick, game pad, satellite dish, scanner, or the like. These and other input devices are often connected to the processing unit 121 through a serial port interface 146 that is coupled to the system bus, but may be connected by other interfaces, such as a parallel port, game port, or a universal serial bus (USB). A monitor 147 or other type of display device is also connected to the system bus 123 via an interface, such as a video adapter 148. In addition to the monitor, computers typically include other peripheral output devices (not shown), such as speakers and printers.

The computer 120 may operate in a networked environment using logical connections to one or more remote computers, such as remote computer 149. These logical connections may be achieved by a communication device coupled to or a part of the computer 120, or in other manners. The remote computer 149 may be another computer, a server, a router, a network PC, a client, a peer device or other common network node, and typically includes many or all of the elements described above relative to the computer 120, although only a memory storage device 150 has been illustrated in FIG. 2. The logical connections depicted in FIG. 2 include a local-area network (LAN) 151 and a wide-area network (WAN) 152. Such networking environments are commonplace in office networks, enterprise-wide computer networks, intranets and the Internet, which all are types of networks.

When used in a LAN-networking environment, the computer 120 is connected to the local network 151 through a network interface or adapter 153, which is one type of communications device. When used in a WAN-networking environment, the computer 120 often includes a modem 154, a type of communications device, or any other type of communications device for establishing communications over the wide area network 152. The modem 154, which may be internal or external, is connected to the system bus 123 via the serial port interface 146. In a networked environment, program modules depicted relative to the personal computer 120, or portions thereof, may be stored in the remote memory storage device. It is appreciated that the network connections shown are non-limiting examples and other communications devices for establishing a communications link between computers may be used.

### Transformations

As noted above, data sometimes is transformed from one form into another form. The terms “transformed,” “transformation,” and grammatical derivations or equivalents thereof, as used herein refer to an alteration of data from a physical starting material (e.g., test subject and/or reference subject sample nucleic acid) into a digital representation of the physical starting material (e.g., sequence read data), and in some embodiments includes a further transformation into one or more numerical values or graphical representations of the digital representation that can be utilized to provide an outcome. In certain embodiments, the one or more numerical values and/or graphical representations of digitally represented data can be utilized to represent the appearance of a test subject’s physical genome (e.g., virtually represent or visually represent the presence or absence of a genomic insertion, duplication or deletion; represent the presence or absence of a variation in the physical amount of a sequence associated with medical conditions). A virtual representation sometimes is further transformed into one or more numerical values or graphical representations of the digital representation of the starting material. These methods can transform physical starting material into a numerical value or graphical representation, or a representation of the physical appearance of a test subject’s nucleic acid.

In some embodiments, transformation of a data set facilitates providing an outcome by reducing data complexity and/or data dimensionality. Data set complexity sometimes is reduced during the process of transforming a physical starting material into a virtual representation of the starting material (e.g., sequence reads representative of physical starting material). A suitable feature or variable can be utilized to reduce data set complexity and/or dimensionality. Non-limiting examples of features that can be chosen for use as a target feature for data processing include GC content, fragment size (e.g., length of circulating cell-free fragments, reads or a suitable representation thereof (e.g., FRS)), fragment sequence, identification of particular genes or proteins, identification of cancer, diseases, inherited genes/traits, chromosomal abnormalities, a biological category, a chemical category, a biochemical category, a category of genes or proteins, a gene ontology, a protein ontology, co-regulated genes, cell signaling genes, cell cycle genes, proteins pertaining to the foregoing genes, gene variants, protein variants, co-regulated genes, co-regulated proteins, amino acid sequence, nucleotide sequence, protein structure data and the like, and combinations of the foregoing. Non-limiting examples of data set complexity and/or dimensionality reduction include; reduction of a plurality of sequence reads to profile plots, reduction of a plurality of sequence reads to numerical values (e.g., allele frequencies, normalized values, Z-scores, p-values); reduction of multiple analysis methods to probability plots or single points; principal component analysis of derived quantities; and the like or combinations thereof.

### Embodiments

The application contains the following non-exemplary embodiments:
Embodiment 1. A method of determining paternity of a fetus in a pregnant mother comprising
   (a) obtaining genotypes for one or more polymorphic nucleic acid targets in a genomic DNA sample obtained from an alleged father,
   (b) isolating cell-free nucleic acids from a biological sample obtained from the pregnant mother comprising fetal nucleic acids;
   (c) measuring the frequency of each allele of one or more polymorphic nucleic acid targets in cell-free nucleic acids;
   (d) select informative polymorphic nucleic acid targets from the one or more polymorphic nucleic acid targets,
   (e) determining the measured allele frequency of each allele of the selected informative polymorphic nucleic acid targets and thereby determining fetal genotypes based on the measured allele frequency for each selected informative polymorphic nucleic acid targets, and
   (f) determining paternity status of the fetus based on the genotypes of the mother, alleged father and the fetus for the informative nucleic acid targets.
Embodiment 2. The method of embodiment 1, wherein step (a) further comprises obtaining genotypes for the one or more polymorphic nucleic acid targets in a genomic DNA sample obtained from the pregnant mother.
Embodiment 3. The method of any one of the preceding embodiments, wherein step (e) further comprises by comparing the measured allele frequency to a threshold of respective polymorphic nucleic acid targets.
Embodiment 4. The method of any one of the preceding embodiments, wherein step (f) comprises determining paternity index for each informative polymorphic nucleic acid targets, determining a combined paternity index for all informative polymorphic nucleic acid targets, which is the product of the paternity indexes for each informative polymorphic nucleic acid targets.
Embodiment 5. The method of embodiment 4, wherein the paternity index is determined by inputting the genotypes of the mother and alleged father and fetal genotypes for each of the informative polymorphic nucleic acid targets into a paternity determination software.
Embodiment 6. The method of embodiment 4, wherein the alleged father is determined to be a biological father if the combined paternity index is greater than a predetermined threshold.
Embodiment 7. The method of embodiment 1, wherein step (c) comprises determining measured allele frequency based on the amount of each allele of one or more polymorphic nucleic acid targets in cell-free nucleic acids.
Embodiment 8. The method of any one of the embodiments above, wherein the informative polymorphic nucleic acid targets are selected by performing a computer algorithm on a data set consisting of measurements of the one or more polymorphic nucleic acid targets to form a first cluster and a second cluster,
   wherein the first cluster comprises polymorphic nucleic acid targets that are present in the mother and the fetus in a genotype combination of AAₘₒₜₕₑᵣ/AB_{fetus}, or BBₘₒₜₕₑᵣ/AB_{fetus}, and/or
   wherein the second cluster comprises SNPs that are present in the mother and the fetus in a genotype combination of ABₘₒₜₕₑᵣ/BB_{fetus} or ABₘₒₜₕₑᵣ/ AA_{fetus}.
Embodiment 9. The method of any one of the preceding embodiments, wherein said polymorphic nucleic acid targets comprises (i) one or more SNVs, (ii) one or more restriction fragment length polymorphisms (RFLPs), (iii) one or more short tandem repeats (STRs), (iv) one or more variable number of tandem repeats (VNTRs), (v) one or more copy number variants, (vi) insertion/deletion variants, or (vii) a combination of any of (i)-(vi).
Embodiment 10. The method of any one of the preceding embodiments, wherein said polymorphic nucleic acid targets comprise one or more SNVs.
Embodiment 11. The method of embodiment 10, wherein the one or more SNVs exclude any SNV, the reference allele and alternate allele combination of which is selected from the group consisting of A G, G_A, C_T, and T_C.
Embodiment 12. The method of any one of the preceding embodiments, wherein each polymorphic nucleic acid target has a minor population allele frequency of 15%-49%.
Embodiment 13. The method of any one of the preceding embodiments, wherein the SNVs comprise at least two, three, or four or more SNVs of SEQ ID NOs: in Table 1 or Table 5.
Embodiment 14. The method of any one of the preceding embodiments, wherein the biological sample in step (b) for is one or more of blood, serum, and plasma.
Embodiment 15. The method of any one of the preceding embodiments, wherein identifying one or more cell-free nucleic acids as fetus-specific nucleic acids comprising applying a dynamic clustering algorithm to
   (i) stratify the one or more polymorphic nucleic acid targets in the cell-free nucleic acids into mother homozygous group and fetus heterozygous group based on the measured allele frequency for a reference allele or an alternate allele of each of the polymorphic nucleic acid targets;
   (ii) further stratify recipient homozygous groups into non-informative and informative groups; and
   (iii) measure the amounts of one or more polymorphic nucleic acid targets in the informative groups.
Embodiment 16. The method of any one of the preceding embodiments, wherein fetal-specific nucleic acids are detected if the deviation between the measured frequency of a reference allele of the one or more polymorphic nucleic acid targets and the expected frequency of the reference allele in a reference population is greater than a fixed cutoff,
   wherein the expected frequency for the reference allele is in the range of
   0.00-0.03 if the mother is homozygous for the alternate allele,
   0.40-0.60 if the mother is heterozygous for the alternate allele, or
   0.97-1.00 if the mother is homozygous for the reference allele.
Embodiment 17. The method of embodiment 16, wherein the mother is homozygous for the reference allele, and the fixed cutoff algorithm detects fetus-specific nucleic acids if the measured allele frequency of the reference allele of the one or more polymorphic nucleic acid targets is less than the fixed cutoff.
Embodiment 18. The method of embodiment 16, wherein the mother is homozygous for the alternate allele, and the fixed cutoff algorithm detects fetus-specific nucleic acids if the measured allele frequency of the reference allele of the one or more polymorphic nucleic acid targets is greater than the fixed cutoff.
Embodiment 19. The method of any one of embodiments 16-17, wherein the fixed cutoff is based on the measured homozygous allele frequency of the reference or alternate allele of the one or more polymorphic nucleic acid targets in a reference population.
Embodiment 20. The method of any one of embodiments 16-19, wherein the fixed cutoff is based on a percentile value of the measured distribution of the measured homozygous allele frequency of the reference or alternate allele of the one or more polymorphic nucleic acid targets in a reference sample set.
Embodiment 21. The method of embodiment 14, wherein the individual polymorphic nucleic acid target threshold algorithm identifies the one or more nucleic acids as fetus-specific nucleic acids if the measured allele frequency of each of the one or more of the polymorphic nucleic acid targets is greater than a threshold.
Embodiment 22. The method of embodiment 21, wherein the threshold is based on the measured homozygous allele frequency of each of the one or more polymorphic nucleic acid targets in a reference sample set.
Embodiment 23. The method of embodiment 21, wherein the threshold is a percentile value of a distribution of the measured homozygous allele frequency of each of the one or more polymorphic nucleic acid targets in the reference sample set.
Embodiment 24. The method of any one of embodiments 1-23, wherein the amount of one or more polymorphic nucleic acid targets is determined in at least one assay selected from high-throughput sequencing, capillary electrophoresis, or digital polymerase chain reaction (dPCR).
Embodiment 25. The method of embodiment 24, wherein detecting the frequency of each allele of the one or more polymorphic nucleic acid targets comprises targeted amplification using a forward and a reverse primer designed specifically for the allele or targeted hybridization using a probe sequence that comprises the sequence of the allele and high throughput sequencing.
Embodiment 26. The method of embodiment 24, wherein the one or more polymorphic nucleic acid targets comprise an SNV, and wherein detecting the amount of an allele of the SNV comprises hybridizing at least two probes to the polymorphic nucleic acid target comprising the SNV, wherein the two probes are ligated to form a linked probe when one of which comprise a nucleotide that is complementary to the allele of the SNV.
Embodiment 27. The method of embodiment 26, wherein the detecting the amount of the allele further comprises hybridizing primers annealed to the linked probe to produce amplified linked probe and sequencing the amplified linked probe.
Embodiment 28. A system for determining paternity comprising one or more processors; and memory coupled to one or more processors, the memory encoded with a set of instructions configured to perform a process comprising:
   obtaining genotypes for one or more polymorphic nucleic acid targets in a genomic DNA sample obtained from an alleged father,
   determining the amount of each allele of one or more polymorphic nucleic acid targets in cell-free nucleic acids from a sample obtained from a pregnant mother,
   select informative polymorphic nucleic acid targets from the one or more polymorphic nucleic acid targets,
   determining the measured allele frequency of each allele of the selected informative polymorphic nucleic acid targets and thereby determining fetal genotypes based on the allele frequency for each selected informative polymorphic nucleic acid targets, and
   determining the paternity status of the fetus based on the genotypes of the mother, alleged father and the fetus for the informative nucleic acid targets.
Embodiment 29. A non-transitory machine readable storage medium comprising program instructions that when executed by one or more processors cause the one or more processors to perform a method of determining paternity status of any one of embodiments 1-27.

### EXAMPLES

The following examples of specific aspects for carrying out the present invention are offered for illustrative purposes only, and are not intended to limit the scope of the present invention in any way.

### Example 1 Work Flow

FIG. 1 shows an exemplary workflow of paternity determination method disclosed herein. Blood (8 mL) is drawn from a pregnant mother into a Streck or Roche cell-free DNA (cfDNA) tube. Cells are removed from the plasma by centrifugation for 10 minutes at 1,000-2,000 × g using a refrigerated centrifuge. The resulting supernatant, which is plasma, is immediately transferred into a clean vial with a sterile pipette. Plasma samples are stored at -20° C. and thawed for use. The plasma samples are processed using bead-based or Qiagen column-based extraction methods to produce isolated cfDNA. Genomic DNA for the mother and any alleged fathers are extracted by conventional methods. Maternal genomic DNA can be extracted from residual buffy coat from the blood sample, and alleged father genomic DNA can be extracted from a blood, buccal, or sport card. 1-5 ng of each genomic DNA is added to the reaction described below.

After DNA extraction, a multiplex PCR reaction is set up with primers that are specific to the SNV panel. The sequences of the SNVs and respective primers (the first primer and the second primer) are provided in Tables 3 and 4. Following PCR, reaction products are diluted and amplified again with a universal PCR that adds on sample-specific barcode sequences. Individual samples are then combined. Because genotyping of genomic DNA and cfDNA sequencing require different read depths for accurate analysis, samples for each can be combined at different concentrations for loading onto the same sequencing cell. Genotyping samples can be added at a 1:10 ratio relative to cfDNA samples.

Combined samples are loaded onto a sequencing instrument such as an Illumina HiSeq or MiSeq sequencer to generate raw sequencing data. Raw sequencing reads are aligned to a reference genome and read counting is performed for each possible nucleotide at the SNV location. The number of reads for each nucleotide at a given SNV is then converted into percent reference allele frequency (RAF) using the formula: reference allele frequency = number of reads for reference allele/ (number of reads for reference allele + number of reads for alternative allele).

For genotyping of maternal and potential paternal genomic DNA, the RAF is used to determine if the individual is homozygous for the reference allele, homozygous for the alternate allele, or heterozygous. Determination is based on a conservative RAF cutoff of 0-0.1 RAF indicating homozygous alternate allele, 0.9-1 RAF indicating homozygous reference allele, and 0.4-0.6 RAF indicating heterozygous. Following this determination, genotypes are uploaded into familias3 open source software for relationship analysis.

For prenatal paternity testing, the mother and alleged father are genotyped from isolated single source genomic DNA using the above method. The sequenced cfDNA is then analyzed differently in order to extract the fetal genotype. First, RAF is calculated for each SNV as above, but these values are then converted to a mirrored allele frequency (mAF). mAF is calculated as the lesser value of the RAF and (1 - the RAF). This mirrors RAF values larger than 0.5 into a range of 0 to 0.5 and groups similar fetal-maternal genotype combinations together. That is, maternal homozygous reference allele SNV/fetal heterozygous SNV groups with maternal homozygous alternate allele SNV/fetal heterozygous SNV. It was discovered that even for loci that are homozygous for a reference allele, where expected frequency for the alternate alleles is 0, the measured frequency for the alternative allele can be above 0, e.g., 0.005. In this example, 0.005 is used as a read cutoff. Next, all cfDNA reads below 0.005 mAF are removed (below 0.005 RAF and above 0.995 RAF). This removes SNVs where only one allele is detected (i.e., fetal and maternal DNA are indistinguishable or fetal DNA is undetectable). Loci where the mother was genotyped to be homozygous are analyzed first. All cfDNA reads at these loci where the mAF is above the cutoff are determined to be loci where fetal DNA is heterozygous. The average mAF for all fetal heterozygous loci is calculated to set the fetal fraction. The heterozygous fetal-specific genotype, maternal genotype, and alleged paternal genotype(s) are then analyzed in familias3. The software produces a paternity index, which represents the likelihood that the alleged father is the biological father based on gentopes of the trio for each informative SNV and a combined paternity index is then determined by multiplying the paternity index for each informative SNV. If the combined paternity index is higher than a predetermined threshold, 10,000, the alleged father is confirmed to be the biological father. If the combined paternity index is below the threshold, the test is inconclusive. If the combined paternity index is 0, then the alleged father is not the biological father.

If the alleged father cannot be excluded, informative SNVs for which the fetus is homozygous and the mother is heterozygous are selected. This can be achieved using maximum likelihood and Bayesian analyses as decribed above to infer the most likely genotypes and assign posterior probabilities to these genotypes. Genotypes with posterior probabilities below a specific threshold (e.g., 99.99%) would be excluded. This will result in more available loci for testing, which will increase the power of the analysis.

### Example 2. Design SNV Panels with Improved Sensitivity

A PCR reaction was set up with primers that are specific to the SNV panels (the sequences of the SNVs and respective primers are provided in Table 3 and Table 4) to amplify the SNVs.

TABLE 3

Panel A SNVs and amplification primers

| SNV | SEQ ID NO | First Primer Sequence | SE Q ID NO | Second Primer Sequence |
|---|---|---|---|---|
| rs38062 | 1 | AAAAACTGCTTGCCTTCTTCTT | 2 | TCTATGGGTTCTCACAACTCAAC |
| rs163446 | 3 | TGGACAAAAATACCATCATCA | 4 | AGATCATCCTGAACATAAGGT |
| rs226447 | 5 | CATCTAAATACATGAAAAAGGAG | 6 | TCAAGTATCCAGGACTTGTTCG |
| rs241713 | 7 | GGACCCAAGATCTGATTCTAGC | 8 | AGGGTGAGCTGTTCTCAGGA |
| rs253229 | 9 | TCCCCAGACTAATTATGGAAAAA | 10 | TCACTTTACTGTTCACCAAACG |
| rs309622 | 11 | GGATTTTAGGGCACTAGGAAGG | 12 | GAGAGTTTTTAAAGAGTGTCGTT |
| rs376293 | 13 | TGTATTTGCCTAAAAGTAAGAGG | 14 | GGCAGAGTTCTCTTGACGTG |
| rs387413 | 15 | CAGCTAAAGGAAAACTATTAATGC | 16 | TCTCTTTGTCTGTTAGGGTTTT |
| rs427982 | 17 | TCATCTGTGAAATAGGGACACC | 18 | GCTCTTAAAACTCATCCCAAGC |
| rs511654 | 19 | AGAAATTATTCAGGACACAGAGA | 20 | TCCTGACAAGACAGTTATCATCT |
| rs517811 | 21 | GAGAAGAATGATTAGACCTTGCT | 22 | ACAAGAGTACACGAGAGAAAAA |
| rs582991 | 23 | TGATGTGGAATAGTTTAGGTGA | 24 | TCCAAAAGGTAATTCCAATATGC |
| rs602763 | 25 | GGATATGCCGCTTTTCCTCT | 26 | GCTAAGTAAATAATTTGGCAGTT |
| rs614004 | 27 | TCACAGTGTTTCTCATAGTTTTA | 28 | CAGCAGCTAGTGTTGCACTAAT |
| rs686106 | 29 | GGTTCACAGAGCCCAAGTTAC | 30 | TGAGTCTCTTACTGATCCTGTGAC |
| rs723211 | 31 | GAGTCACTCTTGGGGTATCA | 32 | GATGCCCAGCCTCTTCTCTC |
| rs751128 | 33 | AGAGATCTCCGCATCCTGTG | 34 | GGGGGCCAATAACTATGCTC |
| rs756668 | 35 | AGTGTGATGTTTGAGTGAGG | 36 | GTCCTATCATCTTTTATTTCCAA |
| rs765772 | 37 | TTCCTTGGCATTTTAGTTTCC | 38 | TCCCATGTAACACCTTTCAGA |
| rs792835 | 39 | TCACCCATTCTTCATACTCTTTG | 40 | AACTTTTCAGGTCGGCAGTG |
| rs863368 | 41 | GGAGAGAATCCCTTACCCTTG | 42 | GGAATTTTATTAGATGTTGAGG |
| rs930189 | 43 | CAGCCCAGATTTTCTCTTTCA | 44 | TCGAGGTAAATAGGCCCACA |
| rs955105 | 45 | TTCAGCTCTTCTACTCTGGACTG | 46 | TGAAACAAGAGAAGACTGGATTTG |
| rs967252 | 47 | GTTATATCTCTTTTGTTTCTCTCC | 48 | TTGGATTGTTAGAGAATAACG |
| rs975405 | 49 | TGGACAAGAGAGACTTCAGGAG | 50 | GCTGAGCCTTTTAGATAGTGCTG |
| rs1002142 | 51 | TCCAACTGGAAAACACCTCA | 52 | GAGCCACCTTCAAGACTCTTTC |
| rs1002607 | 53 | TTTAAATCTTTCCAGGGGGTTT | 54 | TGATTCTCAGCCTGGAGTTT |
| rs1030842 | 55 | AGGATTCAGCCATCCATCTG | 56 | TCTGCCATGGGAGGTATAGA |
| rs1145814 | 57 | AAAACATAATTGAACACCTAGCA | 58 | AATAGGAGGCTGCTCTATGC |
| rs1152991 | 59 | TGATTCACTTCCAGTTCTTGACA | 60 | AGTGACCTTGCTGGTTTGTG |
| rs1160530 | 61 | GGGTACCATATGAGGCCAGTT | 62 | TCTTCTTCCCAATGTCATGGA |
| rs1281182 | 63 | CCAGGCTTCCAAGATTATTGT | 64 | AAGGCATCTCAGGTGTTATTTT |
| rs1298730 | 65 | CCTCGCTGTCCCTGCATAC | 66 | AAGTGCTGACTCTGTTCTGG |
| rs1334722 | 67 | GAATATCTGTCTCGGAATACCA | 68 | GGGATGTGTGATTTCTGAAGG |
| rs1341111 | 69 | GAACAACATCTATCATTCATCTCT | 70 | CACCACTCTAAAGTAGACCATTG |
| rs1346065 | 71 | GCTTTGGGGTTATAGCTGGA | 72 | AGATGGCCATTAGCTAGGAA |
| rs1347879 | 73 | GCACATAGAGGTCTCTCTCTTCT | 74 | CTATATTAGAACACTCAGCAGCTA |
| rs1390028 | 75 | AGGGCTGAACAAGGAACTGA | 76 | CTCATCCTGAGCTCTCGTGTA |
| rs1399591 | 77 | TCACTCATGTTTTACCTTTTAGC | 78 | TGAGTCAGATTCTTCATAACTTT |
| rs1442330 | 79 | TACTGCCAACAGACAACTCG | 80 | TTAGACCGCAGACCTTTAGAA |
| rs1452321 | 81 | GGGGCAGATCAGAAATGTTG | 82 | GGCTGTTCTCAATGGTGTCA |
| rs1456078 | 83 | CCCCATATGTAACCCATCACA | 84 | TCTTTGGAAGAGAAATGTGATTCT |
| rs1486748 | 85 | GGAATGTATTTCTGCTGTGCTG | 86 | TCACTATTCCTTACTCCAGGTGA |
| rs1510900 | 87 | CCATTCACGTGGCACTTTTT | 88 | CACCTTACTGCTTCCTGCTACC |
| rs1514221 | 89 | CCAAAGGCTGTATTATTTATGC | 90 | GTGTTGAAGTGATGTAATTCAG |
| rs1562109 | 91 | TGAACATATCAGCTGGCCATT | 92 | AAAGCCCAGAATTGACTTGG |
| rs1563127 | 93 | CAAACCTCCAGGGTAGTAGACA | 94 | GGGGTTCATAAGGGAAACCA |
| rs1566838 | 95 | TCTCAGAGCAACATGTACCAAAA | 96 | GCCCAATCAGACATCAATCC |
| rs1646594 | 97 | GTTTCCCAGCAAATTCCCTA | 98 | TCATCAAAATGGATCATAACAG |
| rs1665105 | 99 | TTTGGAGTGGGTCTCTTCACT | 100 | AAAGAGTACATTCTGCCTTGCT |
| rs1795321 | 101 | GCTCACTGTTACCCTACTACTCTC | 102 | ACCACACAAATGATTATGGTA |
| rs1821662 | 103 | CCACACACTGAAAAGAATTTGTG | 104 | AGTGGGCTGGATATATGAAAA |
| rs1879744 | 105 | AGGCATGTGTTAAACTAGAAAAA | 106 | GGAGGAAGCTGTGTTCTTTTCA |
| rs1885968 | 107 | GGGGATCTTAAAAGCACCAA | 108 | GACACTCCCACTTCTGCCTA |
| rs1893691 | 109 | CAGCCTAAATTTCCAGTCTT | 110 | AGTTATGAGTAATGAAGGAAGG |
| rs1894642 | 111 | ATTTCTTCAAGTGTATACAGAGC | 112 | CAGGCAAACATTCCCTTGTA |
| rs1938985 | 113 | TGTCTTTGCTCAGTTATGAAGAGA | 114 | TTGTAAATTTTTCTCTAGGTGTG |
| rs1981392 | 115 | GGCATGGCAATACTCTTCTGA | 116 | GATTTTCACATCTAATTTTCACC |
| rs1983496 | 117 | ACAATGAGCTATTTTAACTCCA | 118 | ACTAACTTTGCAAGATACAGATT |
| rs1992695 | 119 | TGGCCACTTGCTTATTTGAA | 120 | TGTTCTTAAGTTGCCCATAA |
| rs2049711 | 121 | CCCACTTTCACAATTTGAATCC | 122 | GAAGAAATACAAAGCAGTTGCTAA |
| rs2051985 | 123 | GCTTAGGAAGGTGTGGAGAGC | 124 | CCACTATTTATGTTTATTGAGTGC |
| rs2064929 | 125 | GAGTCATTTTGTCCACCAACC | 126 | GCTCATAGTTAGAAGTGGCAGCA |
| rs2183830 | 127 | GCAATGATAACAAGAACACAGCA | 128 | TGGAGCCAAAGGGAGTAATA |
| rs2215006 | 129 | TTGCTGGCTTACATTCATTCC | 130 | TACAGCTCAGCCAGTTCTGC |
| rs2251381 | 131 | GAAAGGGATGATGGTTCCAA | 132 | CCCATGAACACATTCACAGC |
| rs2286732 | 133 | GTCTGTCCCTGGGCCATTAT | 134 | CACGATTCAGTAAATGGCTTG |
| rs2377442 | 135 | TGGAGACATGACACTATGAATTT | 136 | CCATCCTGGGATTACCAATCT |
| rs2377769 | 137 | TTCTGTGTTCTACAATGTCTAGGG | 138 | TCATCCATTTGAGTTTTCCAA |
| rs2388129 | 139 | TATGAGCTGTGGCCAATGAA | 140 | CCTGAAGTGTCCCCTAGAAGG |
| rs2389557 | 141 | TTTGCAGACAGGTTAAGATGC | 142 | TGCACCAAGATGTGTTCTGTC |
| rs2400749 | 143 | CCTACAGTCCAGGGGGTCTT | 144 | TCTAGATAAGGAGAATCTGGTG |
| rs2426800 | 145 | CGGAATTGAGCTAACCGTCT | 146 | CACTGGCCTGAGGCTACTTC |
| rs2457322 | 147 | AAGTCCTGGATTTCACCAGAG | 148 | TCCCAAGATCTGCACTAAACG |
| rs2509616 | 149 | CCCTCCAGAGCTAACTGCAT | 150 | TGGATTTATTCTTCATGTTGCTT |
| rs2570054 | 151 | TTTCCAGGAGTATAAAGGAGTGAA | 152 | AACCAACACTTAGGAAAACAAATG |
| rs2615519 | 153 | GAAGCTTCTGTCCCTTCTGT | 154 | CCTGCTGATTTCATCCTTCC |
| rs2622744 | 155 | TCACATCAGTAACCTCCTTCTTG | 156 | TCCAGAAGCCTTTCTTCCTG |
| rs2709480 | 157 | GGCATAGGAACCATATTATTGTCA | 158 | CCTTCTCAACATAGTTCTAATTCC |
| rs2713575 | 159 | CCACAAGCTCATCATCTATTCG | 160 | TTTCTGAGGCTGATAACTGAA |
| rs2756921 | 161 | GAAGGAACATCAAACAAGGAAA | 162 | TGCATATCACAGTCTCCAAGG |
| rs2814122 | 163 | GAGCAGGTAGCTACAATGACA | 164 | TGCCACCCAGATCTCTTTTC |
| rs2826676 | 165 | CCTGATCTGGAAACTCATGAAA | 166 | TGGGGATGTGGGTAAGTTAAT |
| rs2833579 | 167 | GCAACTGGTCTTGTTCCACA | 168 | GCTAAGCCAATGTCTACATCTTC |
| rs2838046 | 169 | TGGTGTGTTAGGGATCTGGAG | 170 | TGACATTGGTTATTGGCAGA |
| rs2863205 | 171 | CGTATTCATTATCCACAGGGACT | 172 | TGCAGTGAAGGATTGCAAAG |
| rs2920833 | 173 | CCCTTCCTGGACTTCACATAG | 174 | GCATCTAGATCTTTACCATTGC |
| rs2922446 | 175 | GGAGAACATTTAGTGCCTCTGC | 176 | ACACTCGGAACGATCTCTGC |
| rs3092601 | 177 | AAACCCACGGAGGTCATTTT | 178 | TGGGTCTCCTATTTCTGTGTCC |
| rs3118058 | 179 | TGTTAGGACTACCTTATGCAGTT | 180 | TGGTATGTCTCCTTTGATCTTT |
| rs3745009 | 181 | CTGAGCGGGAGCTTGTAGAT | 182 | GCTCCTGACGACCAATAACC |
| rs4074280 | 183 | GGACCACTGTCTAGACCAAGC | 184 | TGTGTCTGGTGAGGAAGATGA |
| rs4076588 | 185 | GGGATGAAACCAAACCTCCT | 186 | TTTTAGGAAACCTCACCAGGAC |
| rs4147830 | 187 | TCTCTGTTCGTGTCTCTGTCTTG | 188 | TTGAGTTGGCCTAAAACCAGA |
| rs4262533 | 189 | CCCGACCACTAAAAGGCATA | 190 | TTGCCTCTAAAATCTAGAATAGCC |
| rs4282978 | 191 | TCTTAGGAATGACTCACACTGGTC | 192 | CACTGAATATTGAAAACTAATGG |
| rs4335444 | 193 | GCATGTTATAATTTTACAAGCTC | 194 | TCACACAGGTTAGGATGTTTGTG |
| rs4609618 | 195 | GCACCCTAGGAGCAAACTGA | 196 | GCAGTTGCCTTGAAAGGAGT |
| rs4687051 | 197 | GCAAATAAAATGACTCTGGGAAC | 198 | GGGGTTGAGATACAACATCTTCA |
| rs4696758 | 199 | GATTCTTGGGGCATCAAGTG | 200 | GGACGTGGGTGACTATCAGG |
| rs4703730 | 201 | TCTAGCTCCTAAGTTGATTGATTC | 202 | TCCATTATAGTTCAGTCTTCAAT |
| rs4712253 | 203 | CAGGAGAAAAGCAGAGACCAA | 204 | AGCGAGAGCAGGCTCATAAT |
| rs4738223 | 205 | TGACAAGGGATTAGGGCAAA | 206 | GAAACTACCTCTGAGTGTTACAGA |
| rs4920944 | 207 | GAATCCTGGACGGTCAGAAA | 208 | TGAAAATGAGTAGTGGACATCTG |
| rs4928005 | 209 | AAAATGTGAAGATAAGTGAACAGC | 210 | CCCTAACTTATTCAACATCACTGC |
| rs4959364 | 211 | ACATATTCCAGGAGCATGAC | 212 | CATTGAGTTCATTGGCCTGT |
| rs4980204 | 213 | CTCTCGTGGTGGATTGAACA | 214 | CCAACAAGTACTCTGAACCAATTT |
| rs6023939 | 215 | AAGGAGGGCTTAGCTAGTTG | 216 | GCTCTTTCTCATCTTAAGGCTTC |
| rs6069767 | 217 | GTTAAAATTACTGTTCCAGTTGT | 218 | CAGGCAACCAAATAATAACAAAA |
| rs6075517 | 219 | CCCATTTCCATTTACCGTTTT | 220 | TTGTATTTACAATAGCCATCCA |
| rs6075728 | 221 | TGAAAGTATCAGGAAAAATGGATG | 222 | AGCAGTCAAAGTGAGGATATGTT |
| rs6080070 | 223 | GCAGTAACAAATAACCCCAACAG | 224 | ACCAGCCTTTGTTGTTGAGC |
| rs6434981 | 225 | GGGTTCCAGCAATATTCTACCTT | 226 | GGTAATGAAGAAAGACAAAACA |
| rs6461264 | 227 | TCTAATGCCTCACCAAGCAA | 228 | GCACAGCAGAAACCCAGATT |
| rs6570404 | 229 | CACTAGTCCGGCTTGTGTAAAA | 230 | TGGTGATTACAGAATACCACCAG |
| rs6599229 | 231 | ACAGGAGCGGACAATGAGAG | 232 | TGATGTGCATGTGTCTCAGC |
| rs6664967 | 233 | TGGTCCTCTGCTTCCCTAAG | 234 | CATACATGAGGTGACTACCACCA |
| rs6739182 | 235 | CATCAGATTCCCAACATTGCT | 236 | AGCTCATCCCAATCATCACA |
| rs6758291 | 237 | AAGGGCCATGAGGGTACTTT | 238 | AACCCAAACGTCTAACAAGATACA |
| rs6788448 | 239 | CATCGATAGTATTAGGCCCACA | 240 | TGTGATTTCTTTCTATAGGAGGTT |
| rs6802060 | 241 | GGAAGGAAAGCTCTTTTGGAA | 242 | TTCCAGCCCTGAATAACAACTT |
| rs6828639 | 243 | TGATCATTGCTGTGATGTATT | 244 | AGGATACCATGATTTTGTAGTGC |
| rs6834618 | 245 | CTTCCCTGCACATCCTTTTG | 246 | CTGTTTAGGAAGAGTCATGTAACC |
| rs6849151 | 247 | AACTGTTTTGTCAGCTGCTCAT | 248 | AAAAGACCACTTGATTCAGCTT |
| rs6850094 | 249 | TGAGCACACACATATGGAAGC | 250 | TGCAATGTACATGTGGAGAATC |
| rs6857155 | 251 | CCCGTTCTCCATTCTGGTTA | 252 | CCCAGGGAAGAAAATTGGTA |
| rs6927758 | 253 | TGAAATAGTGCTTATTGCATCG | 254 | AGCCACTCCAGCATTCACTT |
| rs6930785 | 255 | CCACATGTTTCTGAGTGAAGGA | 256 | GGAGTTACAGTTATCAAATGCAGA |
| rs6947796 | 257 | GGAAAGAAGGGAGAATGGTCA | 258 | TTGCATATTCTGGACCTCATCT |
| rs6981577 | 259 | GGAGGCAAAGAAGTTAGGGAGT | 260 | TTTTACCTCCCTGCCCTAGT |
| rs7104748 | 261 | AGGAAATGTAGTCAGGTCTAGGA | 262 | GCAGCTTGAAAACAGCCAGT |
| rs7111400 | 263 | CATGGTAAGTATGCTGTTAAATC | 264 | GCTGAGCAGAAAACATAAGCA |
| rs7112050 | 265 | CAAACCCACACTGTGTTAGCTG | 266 | AGCTAATCTTTGGTACTTCAATCT |
| rs7124405 | 267 | CAAGCATCTTGCTGAATTTCC | 268 | AGTGCAAAGTGAAGATAATGACA |
| rs7159423 | 269 | AGTGTCTGTCTTCCAGTTCC | 270 | CATTCATCCCATCTTCTAACTTCA |
| rs7229946 | 271 | GCAAACATGTAAAGTGTGAGAG | 272 | GCAGTCTTCTGTGATTTTATATT |
| rs7254596 | 273 | CAGAAGGAAGGGGTAAGACACA | 274 | TCCCCTCAGGTAACTTCCATC |
| rs7422573 | 275 | GATTTCTGTGTTGTGCCACAGT | 276 | TTGGTGTCTTACATGTATTGTGA |
| rs7440228 | 277 | GCTGTAGCACATCCAAAAACC | 278 | GAACTGAAAAAGGAATAAAGTAGG |
| rs7519121 | 279 | GGCATAAGCAGATACAGACAGC | 280 | TGAAACCTATAAGCCACTGAGC |
| rs7520974 | 281 | TCCAAAAAGACAGCTGAAAGAA | 282 | AAGCCATGCAGTGGGTATCT |
| rs7608890 | 283 | TCCATACAGGAAGATCCATTAAGA | 284 | GTGCAGTTTGGGCTACAAGA |
| rs7612860 | 285 | TCACACATCATTGGTGAAGG | 286 | AAGTGTCAGAGGGTTAGTGATTCC |
| rs7626686 | 287 | CACCTAAAGATTTCCCCACAA | 288 | GACTTACGGCCTAACCCTTT |
| rs7650361 | 289 | GAACAAGTATACTAGCAAAACGAA | 290 | TTTGTCTAAAGAATTTGACAGTGG |
| rs7652856 | 291 | TCTTGAGAAGCCTTTTCTTACCA | 292 | GCATGAGTGTGTGTCTATGCAG |
| rs7673939 | 293 | TTCTGGACTCTCCACTCTATTTCA | 294 | TGGCATAAGATAGACATATTCACC |
| rs7700025 | 295 | GCATCTATGTCACCAAGCATTT | 296 | GCCGTTAAGCACTGAGCTGT |
| rs7716587 | 297 | TCCACTACTTCTTGGAGTTCA | 298 | TCTTGAATAGCACCCACAAGAG |
| rs7767910 | 299 | GACACTACTGTCCTCAAACG | 300 | GCCCAAAGACCAAGTTTTAGA |
| rs7917095 | 301 | CGTGTCTGTGAGCTCCTTTCT | 302 | AGGTTGTGAAAGACACTGATGG |
| rs7925970 | 303 | TCCAAGCTGTTTCTCATGTTTG | 304 | CAGTGGGCTCACAGTAATGG |
| rs7932189 | 305 | GCAATTCCAGATATCTCTTTAT | 306 | TTATCTACCCATGCTTCTCTC |
| rs8067791 | 307 | AACAGATCACTTACCGCTTTG | 308 | CCCTACATGCATTATCTCCTTT |
| rs8130292 | 309 | TGGTGCCATCCTAGAGTTCTG | 310 | AGTGTGCACTTGCTCATGACT |
| rs9293030 | 311 | CCAGGGATTTCATCTTCACC | 312 | ATGTCTATGCCCTGCCTCAT |
| rs9298424 | 313 | TGTAGTCGAAGCAATGAGATGTG | 314 | TTTCACTCCCTTCTGTATTTAGCC |
| rs9397828 | 315 | AAATGCTTTGCTGCATGTCT | 316 | TCAATGGCAATTTGAGGAGA |
| rs9432040 | 317 | TGAGGAAGTGACAAGTTCAGA | 318 | TTTTCTCCCCATCTGTTACTA |
| rs9479877 | 319 | CAATTTTACATCCAACAGAAGA | 320 | TGGGATTATAAGGAGGTCAAGAA |
| rs9678488 | 321 | TGGTGAGTTTCTTCCCTAGGTT | 322 | CTTGACACCATAGTGGTCACCT |
| rs9682157 | 323 | TTTACTTCTGAGCTGAAGGTACTC | 324 | CACGCAGGCAATAGTAGGAA |
| rs9810320 | 325 | AGCACCAAAGGCAAGTTCAA | 326 | GGATGCCAAGATTGCAAATA |
| rs9841174 | 327 | TTCTTTCTACCCAGGTACTTATCA | 328 | TTTCAAGATGCAAAGGCTTG |
| rs9864296 | 329 | CGAAATCCATAGGACCTACA | 330 | AGCTACACTATTTCCATGTGAC |
| rs9867153 | 331 | CGTCGGTTGTTTTATCATTGC | 332 | GGACAGGTTGTGCATAACTAAGA |
| rs9870523 | 333 | CCTCACTTAAGGAGAACAGTTAGA | 334 | TGCTAATCATCCCTTATTATTGC |
| rs9879945 | 335 | TGACCTACTAGACATCAAGCCTTA | 336 | TGCCAGTAACTTAATCCATAGC |
| rs9924912 | 337 | CCAGACAGGCACATACAGTCA | 338 | GGGAACTGAGTATCTCTGTGTGA |
| rs9945902 | 339 | GAGGTCGAAGTTGTAGGCTTG | 340 | TCAACTTAGTTACAGGTCACACA |
| rs10033133 | 341 | TCAATTTTTGTTGTGGTTTACCT | 342 | AGGTTTTCCTAATAAGACTGCT |
| rs10040600 | 343 | TCAGAGTAGGAATGAACAATTT | 344 | CTCAGGGCCTAAACTTGCAC |
| rs10089460 | 345 | GCACTCATGTGAGTTTGCAC | 346 | CACAGTGAAGTATGTATAAATTGC |
| rs10133739 | 347 | GCCTAGCTGTGCGATTCTTC | 348 | TGATACCAGTTGATGCCACA |
| rs10134053 | 349 | TGACTGAACTCAATTCAAACAGC | 350 | TGGCATCTAGGGTATAGGAAGA |
| rs10168354 | 351 | GGCCACCATCTCCTGTTCTA | 352 | CCTTGTTTGTCTGTATCTGAGC |
| rs10232758 | 353 | CCAACTCTGATTGTGCGACT | 354 | GCTCCAAGCCATAGATCCAG |
| rs10246622 | 355 | GGTGTGTGTATGAGGCTTGG | 356 | AACCGCCAGCATAGCTTCT |
| rs10509211 | 357 | GGTAGGAAGGGGTTGTCGTT | 358 | TTTCTTTCTACTTCTCATCACTCT |
| rs10518271 | 359 | GGACATCAGCACTAACTGAAGTG | 360 | TTCTCTTGTGTGAACCATCCTC |
| rs10737900 | 361 | GCCAGCGTGTAAGACACAAG | 362 | TGGCATTTGTTTACAGACTTATC |
| rs10758875 | 363 | TCCTCCACATTGGTAATTAGGG | 364 | GGTGTCCCCCTCAAATTGTA |
| rs10759102 | 365 | CAAGTTTGTACCTCAGCTTTCA | 366 | TGAGATACTGTTGTCCTCTGC |
| rs10781432 | 367 | TTCCCTTCTTATGTAATCTCC | 368 | GAGGGTTACTGAACTAGGATAATG |
| rs 10790402 | 369 | TCCTGAGAGCATGGTAAGATGT | 370 | TGCAGGGCATTCTATGTGAA |
| rs10881838 | 371 | TACAGCTGAGCAATAACGTG | 372 | TGGCTGGCCAAATCTTTCTA |
| rs10914803 | 373 | AAACTATAAAAGGACCTAGGAAA | 374 | AAGTCTAGTGAATTTCTTGTTAGG |
| rs10958016 | 375 | CTTAATGATTTTGTAATGTCAGG | 376 | ATTTGAGAGGTTGCCAGAGC |
| rs10980011 | 377 | GAGGTTCTCATTCCCTCACC | 378 | AGAGGGGCTCACCTGAGAGT |
| rs10987505 | 379 | CACACTAGTGGGTCCTGATTAGA | 380 | TTGCGGTTTCCTCATTCTTC |
| rs11074843 | 381 | CGTGATGGGTAGGTCAGTCC | 382 | CGCCTCTGGGGATAACTAAA |
| rs11098234 | 383 | GGAATTGCCACTCTGGAGAA | 384 | AGTGGTCCCCAACAACTTGA |
| rs1 1099924 | 385 | ATAACAATGTCTAGCAACAGG | 386 | GATCAACACTTCAAAATTATGGT |
| rs11119883 | 387 | TCAGATAAAACAATTCCAGTTAC | 388 | ACCCACAGAGGAAAGCCTTG |
| rs11126021 | 389 | CAGCATATATTACCTTTTCTTTG | 390 | TGTGCCCAGAAAGTTTTAGCA |
| rs11132383 | 391 | TCAACTGACACTGGTGTTTCTC | 392 | GTGAAGGGAGGACAAAATCG |
| rs11134897 | 393 | CAAGTGATCTGATGGGGTGA | 394 | TGCTGAGTTTGAGAAACTTGGT |
| rs11141878 | 395 | GTAGGACTTAGGGCGCTCAT | 396 | GCATTACTGCCGAGGGATCT |
| rs11733857 | 397 | TGACAAAGCCTAGAGTGAACTGA | 398 | TCCTAGAGTACTCCTCTTTGTCCA |
| rs11738080 | 399 | GTACAGAGTCCCTGTCTCACA | 400 | CATGATCTGTCTCTCTCACTGAA |
| rs11744596 | 401 | GCATTTTCTCACAGCCACAG | 402 | TGGCCTAAAAATTCACCACTG |
| rs11785007 | 403 | AACATTTGCACATTATCAGC | 404 | GCAAGGATCAGTCAGACTACGA |
| rs11925057 | 405 | TGTCCATCAATCTCAAAAGTCG | 406 | CTGATTTCTACCAGTTACTTACCA |
| rs11941814 | 407 | GCATGAGCCACCCTAAATCT | 408 | TGCAGACCATGAGGAATGTT |
| rs11953653 | 409 | AGGATTCCTTATACACTGACCTC | 410 | ACCAAATAATGGTCTACTCCT |
| rs12036496 | 411 | AAGACATTCTCTGCCTTTCTCA | 412 | GGCTCTACTATGGGGAAAATTCA |
| rs12045804 | 413 | GCAAATCACTAGGAAAGCTCA | 414 | GAGGTTCACTCTATTTCTGTTCC |
| rs12194118 | 415 | CTAGAAACGGCTGCCAGGTA | 416 | CCCTGCACTTGTACCAGCTT |
| rs12286769 | 417 | AGGACATTCTTTTGTGTATTCAAG | 418 | ATCCCATATAGGCACTTGCT |
| rs12321766 | 419 | CAAATAATCACCCCAATACAATCA | 420 | GCTTTCAGTGCCCTCATCTC |
| rs12553648 | 421 | AAGATGATCAAAGTTTTGAGAGCA | 422 | CACTCCTAAAGAACAAGATGTCAA |
| rs12603144 | 423 | GACAAGAACTGAAGGCAAAGG | 424 | GGGAGGAACAGAACAACCTTC |
| rs12630707 | 425 | CCCTTGCAATACCCAGCATA | 426 | AGTTATCTGAGTTGGCTTACC |
| rs12635131 | 427 | TCGCAGTCTTTTGCATCATT | 428 | TCCAATAGCTACCTTCACCAGAA |
| rs12902281 | 429 | TGGAAAAACACAGGCATATTCTC | 430 | CCAAAAGCATCTAAAAACAGGA |
| rs13019275 | 431 | CAAATATACTGATTCTGTGGCAAA | 432 | TGATGCATTGAGATTTTGATGA |
| rs13026162 | 433 | TAGCCTTTGGATAACAGTCC | 434 | GAGGGAGGAAATGGTCAACTT |
| rs13095064 | 435 | AGGCAAAGAACTAGACAACTCT | 436 | AGACGTGCTGGGTTCCTAGA |
| rs13145150 | 437 | GGCATGAAGATGTTAACCTACCA | 438 | TTGTCTGGTCTTCATCAAGTCTCT |
| rs13171234 | 439 | TTGCCATGCAGCAGTACTTAG | 440 | TGACTTTTCATTGCTAGTATCCA |
| rs13383149 | 441 | GCAACAAGAACAGGAACCAAG | 442 | TGTTTTGACATTGTCCTGTGTG |
| rs16843261 | 443 | CAGTGAGGTGTGATGTATAAAGAG | 444 | GAGAACACATATTCATTCCTCTCC |
| rs16864316 | 445 | GTGGGGTCCAGCAGTAAATC | 446 | GAACTTCTCACATCACCTCAAGC |
| rs16950913 | 447 | TCTATTAACCCTAATCAATCTCCT | 448 | TTGCTAAATTTCAGGCACCTC |
| rs16996144 | 449 | CCTTTGACTCTGGCCTCATC | 450 | AGTGAATAACCAGCCTTAGTTG |
| rs17520130 | 451 | AAATAAGGACATCTGGAAAACAA | 452 | GTGCCAGCTACAAACAATGG |

TABLE 4

Panel B SNVs and amplification primers

| SNV | SEQ ID NO | First Primer Sequence | SEQ ID NO | Second Primer Sequence |
|---|---|---|---|---|
| rs196008 | 453 | GTGCCTCATCAAAATGCAAC | 454 | ACACAGATGACTTCAGCTGG |
| rs243992 | 455 | AACTCAAACCTAAGTGCCCC | 456 | GGAATGGAATAGTGTGTGGG |
| rs251344 | 457 | ACACTGGTCTCAAGCTCCC | 458 | CACACCTGTAATTCTAGCCC |
| rs254264 | 459 | AGAAGGAAGGATCAGAGAAG | 460 | AGCTTTCCTCCCCACACTG |
| rs290387 | 461 | GCTGTGTGGAGCCCTATAAA | 462 | GAATGAAATGGAGTTTGCAG |
| rs321949 | 463 | CCTCAGCCACCACTTGTTAG | 464 | GTGTTGGTCAGACAGAAAGG |
| rs348971 | 465 | GCCAATTACCCCATAATTAG | 466 | ATGCACACTTACACACGCAC |
| rs390316 | 467 | AAGGAAGTAAAGGTATGTGC | 468 | AGGCTAACTCTAACATCCTG |
| rs425002 | 469 | AAGAGTGTCTCCTCCCTCTG | 470 | AACTGGAGGCTGTGTTAGAC |
| rs432586 | 471 | CGCTCTTTTCTGACTAGTCC | 472 | TTGCAGCAGTCACAGGAAAC |
| rs444016 | 473 | CTCTCTGTGCACAAAAAACC | 474 | GGAAGACACTGCCTTCAAAC |
| rs447247 | 475 | AAAAACCCCAGGCTCCATTG | 476 | ATGTCCAGCTGCTTCTTTTC |
| rs484312 | 477 | TCCAAGTCAGAAGCTATGGG | 478 | AGTCTGCAGACCTAACATGG |
| rs499946 | 479 | ATGGCTTGTACTTCCTCCTC | 480 | TTCGGTGGAATAGCAGCAAG |
| rs500090 | 481 | CATAATCTCAGGGCTACAT | 482 | TTCACCTGGCCTTGAGGGTC |
| rs500399 | 483 | GTTTATTGATGAACTGGTGC | 484 | GGGCAGAGTGATATCACAG |
| rs505349 | 485 | ACTGGCAAGTCCAGGTCTTC | 486 | AAGGCTCAGGGCAGAAGCAC |
| rs505662 | 487 | TCCTCATCCGGTGTGGCAA | 488 | CAGCAAAGAGAGAGAGGTT CC |
| rs516084 | 489 | AGTATGCCATCATGAAAGCC | 490 | CTTCTTTGACTAAGGCTGAC |
| rs517316 | 491 | CTCTGCCTATTCTCCTCTTC | 492 | TAGACCTCAAGGCCTAGAGC |
| rs517914 | 493 | AGTAAGAGCTCCCTTGGTTG | 494 | GCTCATAACAATCTCTCCCC |
| rs522810 | 495 | TCCCCTCTACCCCTTGAAGC | 496 | CAGCACTGATGACATCTGGG |
| rs531423 | 497 | AAGAACACAGGCCTGGTTGG | 498 | TATGGCTCTGGGGCTCTATA |
| rs537330 | 499 | AACAGAGAGAATGAGGAGGG | 500 | TCATTCTAAAAGGGCTGCCG |
| rs539344 | 501 | GAAAGGTATTCAGGGTGGTG | 502 | GATGCTCTGAGACAATCCTG |
| rs551372 | 503 | TTAACTGTGAGGCGTTCACC | 504 | GATCATGGGACTATCCACAC |
| rs567681 | 505 | CCAGCCCTGCTCCTTTAATC | 506 | GGAGAAGATCCTACACTCAG |
| rs585487 | 507 | CCAACTTCTTCCCAGTCTGT | 508 | CTGGAGCTGAAGGACCCCA |
| rs600933 | 509 | GGAGAAATCCTTCCCTAGAG | 510 | TTCAAGGTGCTGCAGGTTTG |
| rs619208 | 511 | CCCCCTCTACAGGAAAATTC | 512 | TTCTGAATTCTTCAGCCAGC |
| rs622994 | 513 | CATCCTACCTCTAGGTACAC | 514 | GGTGTCTTAGTTACATGTGC |
| rs639298 | 515 | TGGTGACGCAAGGACTGGAC | 516 | ATACTGTGCTGCTCTTCAGG |
| rs642449 | 517 | CAGCTGCTGTTCCCTCAGA | 518 | CCAAAAAACCATGCCCTCT G |
| rs677866 | 519 | TAATTGGTACAGGAGGTGGG | 520 | AGGCATGGGACTCAGCTTG |
| rs683922 | 521 | GTGCAGGTCATTGTGCTGAG | 522 | AAACACTCCACGTTAAAGGG |
| rs686851 | 523 | CAGCTGAGAAAACTGAGACC | 524 | TTTACAGACTAGCGTGACGG |
| rs870429 | 525 | TGCTGCTCCGCCATGAAAGT | 526 | ATGCAGGGAGAGCAGCAGCC |
| rs949312 | 527 | GCTGAGAGTTAAGTGGCCAA | 528 | CTGTGGCCATATTTCTGCTG |
| rs970022 | 529 | GCAATCAGGCCCAGCTTATG | 530 | TTGTCTGGACTCTCTTCATC |
| rs985462 | 531 | CGCCTAATTTCCAGCAAGAA | 532 | GACTTGCAAAAGCTCTCTGG |
| rs1115649 | 533 | GTCTGGCTGAGGAATGCTAC | 534 | AAGGGCAGCATGAGCTTGGG |
| rs1444647 | 535 | GTCTACTTCAAATCATGCCTC | 536 | CTACATGCATATCTGGAGAC |
| rs1572801 | 537 | CAGAGATGCAAGCAGCCAAG | 538 | AGGAATGGGGCTGCCATCT |
| rs1797700 | 539 | GAGACAGGCAAAGATGCAAC | 540 | ACCACGCCTGGCCAGAACT |
| rs1921681 | 541 | GGGTTTAGTCTCCTTACCCC | 542 | AATGTCCCTGGCACAGCTCA |
| rs1958312 | 543 | GCTTCAGTTGTCACTGTGAG | 544 | CTCAGATGATGTCCCTTCTT |
| rs2001778 | 545 | CGATGCAAGCTTCCATTCTA | 546 | GGACAGAGAATGGCCTGCTA |
| rs2323659 | 547 | TTAAAACAGCCCTGCAACC | 548 | TGATGAGAACAGAGCTGAG |
| rs2427099 | 549 | CTGAAGCTATGTCCTGTTAG | 550 | AGGTGGCACGGCACGTTCAT |
| rs2827530 | 551 | CTGAAGTGCAGGAAGCTTGG | 552 | ACCCTAGAACTTGACACTGC |
| rs3944117 | 553 | AAGGAGCTGGCAAGGCCCTA | 554 | ACATAGGCACAATGAGATGG |
| rs4453265 | 555 | TACCTTTCAAGCTCAAGTGC | 556 | TTTGGATGGAACGTTTGCAG |
| rs4745577 | 557 | GCTACCCTTTAATGTGTCTC | 558 | ATGAAGAGCAGCTGGTCAAC |
| rs6700732 | 559 | CAGCCCTTGTGTGCATAAAG | 560 | TACAGTGGTGGACAAGGTGG |
| rs6941942 | 561 | CTTGTTTTGCAGGCTGATTG | 562 | TCAATCATCCCCATCCCCAC |
| rs7045684 | 563 | GCACATCACAAGTTAAGAGG | 564 | CCCCAGTAGGGAACACACTT |
| rs7176924 | 565 | CAGGATGCACTTTTTGGATG | 566 | GGCTTCTCCCAGAAAATCTC |
| rs7525374 | 567 | ACTGCAGTGCCGGGAAAAGT | 568 | TTTGCTCACCCTACCCCAC |
| rs9563831 | 569 | TGATAACAGCCTCCATTTCC | 570 | TAGGGATGCAAGATGAAAGG |
| rs10413687 | 571 | GATGCAGGAGGGCGTCCCA | 572 | TCCAGCCACTCTGAGCTGC |
| rs10949838 | 573 | TCTGCTGTTTGATGGATGTG | 574 | TGGGAGATCAGCTAGGAATG |
| rs11207002 | 575 | GCTGGGATCCCATCTCAAAG | 576 | TGAATGTCTTGCTTGAGACC |
| rs11632601 | 577 | TTCCCTTGTTTGGAACCCTG | 578 | CAGCTTCCACCCTCTCCAC |
| rs11971741 | 579 | TGGCCTTAAACATGCATGCT | 580 | GGTGACAATCTAGAGAGGTG |
| rs12660563 | 581 | AGGTCAGCTCAGGGTGAAGT | 582 | GCTCCATTGAAGGGTAAAGG |
| rs13155942 | 583 | GAGGGTACCTTTCTTTCTCC | 584 | GCTCAGTGTCTGACAAAAGC |
| rs17773922 | 585 | AGCCATGTTTCAGGGTTCAG | 586 | CAGTGCCTGACAGGGAAAGT |

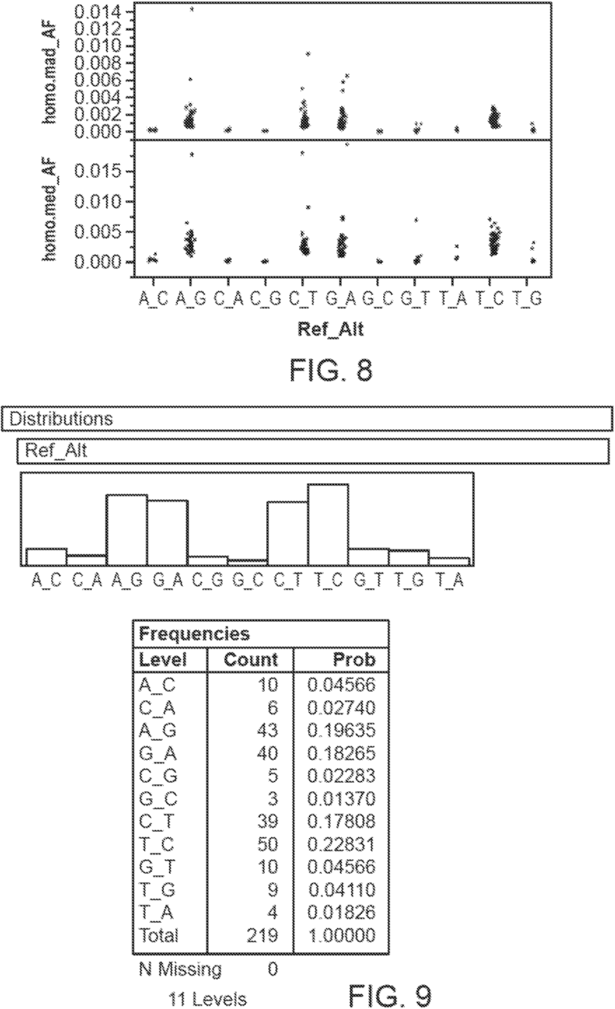

During characterization of the SNV panels above, it was determined that certain categories of SNVs had higher amount of bias and variability in their allele frequencies. For a homozygous SNV, the allele frequency should be equal to 0 or 1. Background is defined as a median bias away from 0 or 1. This is caused in part by sequencing error or PCR error. The variability is the median absolute deviation (MAD) of the homozygous allele frequencies - in an error free measurement, this would be 0. When these biallelic SNVs are categorized by their combinations of reference and alternate alleles (abbreviated as Ref_Alt), it is observed that A_G, G_A, C_T, and T_C have the highest median and MAD for homozygous SNVs (FIG. 8) and represent 78.5% of the panel (FIG. 9). These Ref_Alt combinations serve as a lower limit to the fetal fraction that can be detected.

This motivated the development of a v2 panel that has only lower background Ref_Alt combinations in order to improve sensitivity for low levels of fetal fraction. The v2 panel retains 47 SNVs from the v1 panel and adds in 328 new assays that all have the desired Ref_Alt combinations (not any of A _G, G_A, C_T, or T_C).

The first step in the design process was to identify SNVs that can serve as a universal individual identification panel. The goal was to be able to distinguish fetal DNA from maternal DNA regardless of the population (e.g. Asian, European, African, etc.). The ALlele FREquency Database (ALFRED, site: http://afred.med.yale.edu/afred/sitesWithfst.asp) provides allele frequency data on human populations.

The Fixation Index (FST) is the proportion of total genetic variance contained in a subpopulation relative to the total genetic variance. A low value is desirable for obtaining a SNV that will have similar genetic variance in most populations. The first step in panel development was to filter this database to obtain SNVs with a FST lower than 0.06 based on a minimum of 50 populations. The SNVs were further filtered to ensure a minimum average heterozygosity of 0.4 (the maximum possible is 0.5). This increases the proportion of SNVs in the panel that will be “informative,” increasing the confidence in the measurement of donor fraction. This filtering resulted in 3618 SNVs.

FASTA sequences were obtained for these SNVs from dbSNP (site: Error! Hyperlink reference not valid.ncbi.nlm.nih.gov/projects/SNP/dbSNP.cgi?list=rslist). On average, this provided a 1001 bp flanking sequence that included the SNV plus 500 bp both upstream and downstream of the SNV. These sequences were used in the primer design tool BatchPrimer3 (site: Error! Hyperlink reference not valid.probes.pw.usda.gov/batchprimer3/) along with the following parameters to obtain candidate primers for each SNV.:
Product size Min: 40; Product size Max: 54;
Number of Return: 1; Max 3′ stability: 9.0;
Max Mispriming: 12.00; Pair Max Mispriming: 24.00;
Primer Size Min: 18; Primer Size Opt: 20; Primer Size Max: 24;
Primer Tm Min: 52.0; Primer Tm Opt.: 60.0; Primer Tm Max: 64.0; Max Tm Difference: 10.0;
Primer GC% Min: 30.0; Primer GC% Max: 70.0;
Max Self complementarity: 8.00; Max 3′ Self Complementarity: 3.00;
Max #Ns: 0; Max-Poly-X: 5;
Outside Target Penalty: 0;
CG Clamp: 0;
Salt Concentraion: 50.0;
Annealing Oligo Concentration: 50.0.

Processing through BatchPrimer3 resulted in 2645 assays that met the design criteria. These SNVs were further filtered based on additional characteristics obtained from the dbSNP database. SNVs were selected if they met all of the following criteria:
1. Biallelic.
2. The SNV is not located within the primer annealing regions.
3. Validated by the 1000 Genomes Project.
4. The ref_alt combination is not any of A_G, G_A, C_T or T_C.
5. minor allele frequency is at least 0.3.
6. The sequence for amplified target region is unique and cannot be found elsewhere in the genome.

The result is a 377 plex panel that includes 2 assays for total copy calculation and 375 assays for fetal fraction measurement. The fetal fraction assays consist of 47 primers from the v1 panel and 328 newly designed primers. This panel was further filtered to obtain a 198 plex (2 for total copies, 196 for fetal fraction) (Table 5) after removing assays with low depth, high allele frequency bias (deviation from 0, 0.5, or 1 in a test with pure samples), or having a significant role in lowering the alignment or on-target rate (determined from re-aligning unaligned or off-target reads to first 18 bp of each of the primers). Table 6 lists the excluded SNVs and provides reasons for their exclusion. The first primer and the second primer were used as a primer pair to amplify the region containing the SNV in the same row in Tables 5 and 6.

TABLE 5

SNV panel and amplification primers

| SNV | SEQ ID NO | First Primer Sequence | SE Q ID NO | Second Primer Sequence |
|---|---|---|---|---|
| rs150917 | 587 | CTGTTTTCTCAGAAGGGACTTT | 588 | TCGAAAGAAAACACTGAGAATCAA |
| rs163446 | 589 | TGGACAAAAATACCATCATCA | 590 | AGATCATCCTGAACATAAGGT |
| rs191454 | 591 | TTCCCTCTTCAGTTTACCTGTTT | 592 | CACCAAGAAGGGAATGAAAAT |
| rs224870 | 593 | TGAAGAAAGCAAGGGACAGAA | 594 | AAGCCGCGTGTTATTGAAAC |
| rs232504 | 595 | TTCAGTGCTTTCCGTTGGA | 596 | CACACACACGCACTAAGCAA |
| rs258679 | 597 | TCACCTCATACATGTTTTCTTTT | 598 | AATACCTCAAAGGACTGTAATG |
| rs260097 | 599 | TGCTGCATTCATTTGTCAAC | 600 | GAACTCTGGTGTTCCTAGTG |
| rs376293 | 601 | TGTATTTGCCTAAAAGTAAGAGG | 602 | GGCAGAGTTCTCTTGACGTG |
| rs390316 | 603 | AAGGAAGTAAAGGTATGTGC | 604 | AGGCTAACTCTAACATCCTG |
| rs468141 | 605 | ACTTAAAACCAAACCCTCA | 606 | TTATTGGGTGTTGCAAGTGT |
| rs500399 | 607 | GTTTATTGATGAACTGGTGC | 608 | GGGCAGAGTGATATCACAG |
| rs522810 | 609 | TCCCCTCTACCCCTTGAAGC | 610 | CAGCACTGATGACATCTGGG |
| rs534665 | 611 | ACGGGGTCTTATGGTTCCTC | 612 | GCCTGAGAAGCAATTAACCTG |
| rs535468 | 613 | TGCTAACCTGTGAAGTCCATTC | 614 | TTTATTTGCATTGGTCTTTGC |
| rs535689 | 615 | GCATAATTTGAAAGCTCTGTTTG | 616 | CGATTATGCCCATTGATATTTTT |
| rs535923 | 617 | TCAAGGGATTGCTCCAATGT | 618 | CTCCAAACCAATACCTAAAAA |
| rs567681 | 619 | CCAGCCCTGCTCCTTTAATC | 620 | GGAGAAGATCCTACACTCAG |
| rs570626 | 621 | GCTTCTCATCTGTGTGCATTT | 622 | CCTAGAATATGATGCCCAAACA |
| rs580581 | 623 | CCTCCTCTACTAGACCTCTGACG | 624 | TGTAGAATAAGAAGGCAGTCCAA |
| rs600810 | 625 | ACCTAGGGAAGGGGTCAC | 626 | AAGCCAGGGTTCATCTGC |
| rs622994 | 627 | CATCCTACCTCTAGGTACAC | 628 | GGTGTCTTAGTTACATGTGC |
| rs698459 | 629 | TCCAAAATTCCTTGATGTGTCA | 630 | TCAACCTCCTACAGCAACAAAA |
| rs707210 | 631 | GGTTCACTACAGAGCGTCTCAA | 632 | ATGTACCTTTTGGGCCTTGC |
| rs729334 | 633 | CCACCAACCTGCCTCTGG | 634 | TGATTTGTGATCAGTCTTCCTCTT |
| rs747190 | 635 | ATTCTTCCTCCTGCAATCCA | 636 | TTTGGAAGTCGGTGCTAACC |
| rs751137 | 637 | GGCTTGCTTAACATGTGCTG | 638 | CAAAGATTGCAGATAAAGTGCT |
| rs765772 | 639 | TTCCTTGGCATTTTAGTTTCC | 640 | TCCCATGTAACACCTTTCAGA |
| rs810834 | 641 | TTTGCATTCTCCTGTCTCTTTTT | 642 | GGAACCACTACAGGAAACGAA |
| rs827707 | 643 | TTTTGCCAAGCTATTCACAG | 644 | CTCCATCGAGGGATTATCAGA |
| rs876901 | 645 | GCACCTATTCACAGACAGTTTGA | 646 | AGAATCTTCCGATTCTGCAT |
| rs895506 | 647 | GCCCCTATAATCCTTGGAGTC | 648 | GAGGAGCCAAAGAGCTGAAA |
| rs930698 | 649 | GGTTTCATTACTCTATGCTTCTTC | 650 | AGGAGATGTGCATTTCAGCA |
| rs937799 | 651 | CAGGACAGGAATTAGTGTTGC | 652 | TTTTAAATACTACGGAGTCAAAC |
| rs955456 | 653 | GCCCTTGAAAAGAGGGCTTA | 654 | GCAGGATATTCTCTGACTGCAA |
| rs974807 | 655 | AAAGAGTATAGGGATGGACACTGA | 656 | CGTGTAGTAGTCACCCGGTTT |
| rs994770 | 657 | GAAAGCCTACACGCCCAAG | 658 | TTTTCAGTGTCCTCACCTCTGA |
| rs1002142 | 659 | TCCAACTGGAAAACACCTCA | 660 | GAGCCACCTTCAAGACTCTTTC |
| rs1017972 | 661 | CAAAATTTCCAGCGCATTCT | 662 | ACTGATTCCTCGCAGCCTTG |
| rs1057501 | 663 | ACTGCATTGTGGCGGTATCT | 664 | AAAAGTACATGATGCATTTAAGC |
| rs1145814 | 665 | AAAACATAATTGAACACCTAGCA | 666 | AATAGGAGGCTGCTCTATGC |
| rs1278329 | 667 | CGCTGGTAAATACTTAGAGATAAA | 668 | ACATGTTCCCCATTGCTCA |
| rs1336661 | 669 | CAGTCTTGTTGTATTCCCTAAAGA | 670 | GCAACTGAGAGGATGAGGTTG |
| rs1340562 | 671 | GACCTAAGACTAGTGCCGTGAA | 672 | GTGCAAAGGAAACCAGGAGA |
| rs1356258 | 673 | GGAATAATATATGTGGACTGCTT | 674 | TTACCCTTAAAAATTCCTTGG |
| rs1396798 | 675 | AAAGCAAATGGTTAAATAGCAGA | 676 | TTGGTTCTTTCTCTTTAATTGTG |
| rs1406275 | 677 | CAGAGAGAAAGCAGTTTGAATTTG | 678 | CCAAGATACCTTGCCTTCTGA |
| rs1437753 | 679 | CATCATATTCCTAACTGTGCTCAT | 680 | TCCTTGGTAAAGAGGGTAAAGAAA |
| rs1442330 | 681 | TACTGCCAACAGACAACTCG | 682 | TTAGACCGCAGACCTTTAGAA |
| rs1444647 | 683 | GTCTACTTCAAATCATGCCTC | 684 | CTACATGCATATCTGGAGAC |
| rs1482873 | 685 | ACTGAGGAGTAATTCATGAGG | 686 | TGGTTTTACCTTTCTGAAAAACA |
| rs1512820 | 687 | CACCTCCTAAGACAAAATGGCTA | 688 | CCTAATCCAGCAGACCATGT |
| rs1517350 | 689 | GGAGGCAGAAATTGCATCAG | 690 | GCATAGCCAGCCATTAGCAT |
| rs1566838 | 691 | TCTCAGAGCAACATGTACCAAAA | 692 | GCCCAATCAGACATCAATCC |
| rs1584254 | 693 | CCTCAAGGCCTCTCCATTG | 694 | GAAGAGTTTTGACTTTTTCTGAGG |
| rs1610367 | 695 | ATCCCCAAGCCCAAGAAG | 696 | ACAGCCATGAACGAAGCATT |
| rs1714521 | 697 | GGCTCATGAACTAAGATAGTTTGG | 698 | AAGAAAGATTGTGGGATTAGACA |
| rs1769678 | 699 | CCATCAGAGCTTAGGGTTGAA | 700 | TTGGAGGAGAAAGGCATCAG |
| rs1979581 | 701 | CCATCTTAGTTGGAAATAGCAACC | 702 | CCATCTTCTTTTCCCAAGCA |
| rs1990103 | 703 | ACATGCTCCTAGGGTGCTTC | 704 | TTCTTGACGGTGTTCTGTTTTT |
| rs2004187 | 705 | CCCTTGTTGGGGAAATAACA | 706 | CCCTATTTCCTACTGAACGCTTA |
| rs2010151 | 707 | TTGGAATGTCCATCCTTTGAG | 708 | CAAACCCATGGCCTTGAA |
| rs2022962 | 709 | GGTATGTATGTGGGAAGGGAAT | 710 | AAGGTTATGTAAGAAAGATGTCA |
| rs2038784 | 711 | AAGGAAGAATTCTCAATGACCT | 712 | TGGGGCTAAAAGTCAGACCA |
| rs2040242 | 713 | TTTAAGATATGCTCTCTCCTGACT | 714 | CTATTAGTTAGGTTTCCAGTTGA |
| rs2055451 | 715 | AGGAAATCTGTGAGTAACTATCAT | 716 | CCTAATAGACCTAACAAGGATGC |
| rs2183830 | 717 | GCAATGATAACAAGAACACAGCA | 718 | TGGAGCCAAAGGGAGTAATA |
| rs2204903 | 719 | TCTCTCCACCTTTCCACACTG | 720 | TGTGTGAAACCTGTGACTTGC |
| rs2244160 | 721 | CATATTCATACCTTCAAGCCAAC | 722 | TGTGGAAACACAGCCCATT |
| rs2251381 | 723 | GAAAGGGATGATGGTTCCAA | 724 | CCCATGAACACATTCACAGC |
| rs2252730 | 725 | CAGGAACTCGCTGAATACCC | 726 | CAGAGGAGCACCAGCCTATG |
| rs2270541 | 727 | GCCATGAATTAGGAGCCTTG | 728 | CAATCCAACGAAGATGACCA |
| rs2291711 | 729 | ACCATGACCTGGCTTGAAGT | 730 | GGACGATCAGGTTACACCTAA AA |
| rs2300857 | 731 | TCCACCTCCTAACCAAGGAC | 732 | CAGCTGAACACTGAGATTTTT |
| rs2328334 | 733 | AAGCCCTGTTTCCCTGTTTT | 734 | CATCTGCAGAAGACAGACTC |
| rs2373068 | 735 | ATCATTCCCGGAGCTCACA | 736 | GACACAATGTGCCTTGAAA |
| rs2407163 | 737 | GTACAGCTGGAATGGCCAAG | 738 | CCCAGTTTCCATCCTCAGTC |
| rs2418157 | 739 | AACAATTTGCTCTGAGAACCTC | 740 | TCTTGGCCTTCAGGGTTTC |
| rs2469183 | 741 | CCTTTGTTACTAAGAATTGAAGTG | 742 | TCGTTTCTTATTGTCTTCTGTT |
| rs2530730 | 743 | CTCCCAATATCCGACAGCTC | 744 | CCACCTCAGGACAGGAGAGT |
| rs2622244 | 745 | TGGATTGATGGCAGAACATT | 746 | CTGAGGGCTTTTTGGCTAAC |
| rs2794251 | 747 | TTTTATTTTTCTCACAAGCCTGA | 748 | TCAGAGAGATAAAGAAGGAAAGGA |
| rs2828829 | 749 | TCTAATTAAGCCATGACTCC | 750 | GGCTGTGGTATGGCTAGCAG |
| rs2959272 | 751 | CACAGAGAAAGAACAGAATCTGAA | 752 | AGGCAGACAGATGGACACAT |
| rs3102087 | 753 | GAGCTTTGCATGCAGTAGGG | 754 | CCCAGCCTCTCTGTCTATGG |
| rs3103810 | 755 | TGACTTCTATCACCCCTACC | 756 | GTGCAGGAGAGGAAAGCAGA |
| rs3107034 | 757 | GTTGATGACACCCACATTCA | 758 | GCACGACGTACGAATGAGTC |
| rs3128687 | 759 | AGCACCAGGCTTTGGCTAT | 760 | GAAGGATGTGAGAAAAGACCTG |
| rs3756508 | 761 | GCATGGTCACTGAGTTTTGC | 762 | CAAGCCACAAGAGGTGATGA |
| rs3786167 | 763 | CACAGAACAGCTTGTGAAAATCA | 764 | TGGTACTAAGACCCACCAAAA |
| rs3902843 | 765 | AAAACCCTCTAACTAGGCATT GAA | 766 | GCTTGCTCTTATTATTTTGACGTT |
| rs4290724 | 767 | AGAATTTGGAACTCACTTTGG | 768 | AAACAGATCCTATTGTGTCTGGAA |
| rs4305427 | 769 | ACCTCATGCACCAGCCCTTA | 770 | AAGTGTTGCTCCCTGCTGTC |
| rs4497515 | 771 | AAAGGTCTTTCAGGAGAATTTG | 772 | AGGTGGCCATACACATGCTT |
| rs4510132 | 773 | GGTTGTCCATGTCCCCAAG | 774 | TTTGCAGTGTTTATGCCACA |
| rs4568650 | 775 | TCATGGCAATTTAAATGATGAG | 776 | TTTAAATGGTGCCTTGTTTCTT |
| rs4644241 | 777 | CAGGGCACTAACTGAAAAAT | 778 | GGGATATGGATTATCTTTCTCAT |
| rs4684044 | 779 | AGCCCCAAACTAAGTGCTGA | 780 | CCCAGAGCCAGTGCATTTA |
| rs4705133 | 781 | TGATGAGAAAACACAGAAATGC | 782 | CCTGGCTGAATCAAGGAAGA |
| rs4712565 | 783 | CAGTGACAGTTTTCTCATTAAGC | 784 | TAGGAACAATCCCCAATCCA |
| rs4816274 | 785 | TGAGAAACTCACTTGGGGTCA | 786 | TGACAGCAATTCTGGTCTGC |
| rs4846886 | 787 | AGGCTTGAAGAAAAGCTTCAT | 788 | CTTTTTCATATCCAGTATTTCAG |
| rs4910512 | 789 | CAGCTAGAATCTATACAAGGAAGG | 790 | GGATACAACAGGAACTAGGATCAA |
| rs4937609 | 791 | CCCATTATTATGCTGTTATGCTG | 792 | TCTGAGAGTTAAATCCTTGGTGA |
| rs6022676 | 793 | CACCTCTTAACAGTTTCATTTT | 794 | GGCCGACAGCTTCTACTTTA |
| rs6023939 | 795 | AAGGAGGGCTTAGCTAGTTG | 796 | GCTCTTTCTCATCTTAAGGCTTC |
| rs6069767 | 797 | GTTAAAATTACTGTTCCAGTTGT | 798 | CAGGCAACCAAATAATAACAAAA |
| rs6102760 | 799 | GGATTCTGCAGACCCTCAGT | 800 | CACCTTGCCACTCACTGTTG |
| rs6434981 | 801 | GGGTTCCAGCAATATTCTACCTT | 802 | GGTAATGAAGAAAGACAAAACA |
| rs6489348 | 803 | CTGTGTGGCTGGGGAAGC | 804 | GCACATAACCTCAGAACCAG |
| rs6496517 | 805 | GGAGCCCCAACCCTAATTT | 806 | ATCCTCATCCTCCGCACA |
| rs6550235 | 807 | CGGTAGCTAAGTATCTGCTTTTT | 808 | GGGCAGGAATTATTATGTTCCA |
| rs6720308 | 809 | GGATGTTTTTGCAGTTTATT | 810 | ACTTGCTCTGATACCTAAATGA |
| rs6723834 | 811 | CGGCTCTCTCCTCATTCTGT | 812 | GCATTGCCACTGAGACATGA |
| rs6755814 | 813 | AAGAGGAGGGCTTTGAGTCC | 814 | TTTAGTAGAGCTACTGATCATTCC |
| rs6768883 | 815 | CAATTAAGTCAGGTAATAATGCTG | 816 | AAGCCATTCATTTGGGTTTG |
| rs6778616 | 817 | TTGATTCCTATTGAGCTTTCA | 818 | GGCCTCTGACATCACTCTCA |
| rs6795216 | 819 | GGCAAGGGTTTAGGACTTGG | 820 | GGATTGCGCCTCAAAATAAA |
| rs6834618 | 821 | CTTCCCTGCACATCCTTTTG | 822 | CTGTTTAGGAAGAGTCATGTAACC |
| rs6840915 | 823 | TGGCCTATTTCTCAAATGCAG | 824 | CTGCAAGGCACGATCTATGA |
| rs6848817 | 825 | GTGATTCTAACAGGTATGTAATGA | 826 | TGCATGTTAACACCACATTGAG |
| rs6872422 | 827 | GGAGACCATACTGAAGTTATTTT | 828 | TTTCGAGTTGGTGGTAATTT |
| rs6902640 | 829 | TCGAAGGTAGAATTAAATGTTTC | 830 | GATAGTGACTTATAACAACTCCAA |
| rs6979000 | 831 | TGAATTGAAGGGTTTTGGAC | 832 | GCACACGTTAAGATGGTTTGAA |
| rs7006018 | 833 | GGGGAGGGAGACGTAAAAAC | 834 | TCCAGATTTTCCTGTTCATGATT |
| rs7045684 | 835 | GCACATCACAAGTTAAGAGG | 836 | CCCCAGTAGGGAACACACTT |
| rs7176924 | 837 | CAGGATGCACTTTTTGGATG | 838 | GGCTTCTCCCAGAAAATCTC |
| rs7215016 | 839 | GGGGAGGCCCTACAAGTTAT | 840 | GAAGGGAGGGGCATCTTTA |
| rs7321353 | 841 | AAAATCACATCTGCTAAATATCC | 842 | TGGACGATAGAACTTGTTAGTGC |
| rs7325480 | 843 | CCATTAAGCAGACACACCTACG | 844 | CTCCTTTGAAAGTGGATCAAA |
| rs7539855 | 845 | TCTGAAAATGGGGCTAAAACTT | 846 | TCCTTAAAGCAGCCCTAAAA |
| rs7568190 | 847 | AGTTTAGATTTCAGTCTATGCAA | 848 | TGGAGAATAGCTCCTGCAGTT |
| rs7580218 | 849 | TCTTTCTGGAGACACTCAGG | 850 | CTGGAATCTAGAAAGAAAAAGAA |
| rs7609643 | 851 | CAAAGATAGATGAGATGCTTTT | 852 | CTGACATTGAAAACTTGAAAGAA |
| rs7632519 | 853 | AGCCCTCCTCCACCGTTAG | 854 | GCCCAGCTACGATTTCTCCT |
| rs7660174 | 855 | TTTTATGCAGCCTGTGATGG | 856 | CCCTTAGTTCAATCAAGCCAAC |
| rs7711188 | 857 | CACTCTTGCAATCTCCCTCAG | 858 | CTGACCCTTGTGGGATTCAT |
| rs7765004 | 859 | CTTTTATGATATCCACCAAGACT | 860 | TGGATCATCTGTCCAAAGTCA |
| rs7816339 | 861 | CCAAAACCTGCTCTCCAAGA | 862 | AAGACTACTGAGGTTGTGCAAAGA |
| rs7829841 | 863 | TTCAACTTGGTACCCTGAAAAA | 864 | AGTCAGTTAGTATGCAGTACTTGG |
| rs7916063 | 865 | TCTTAAAAGTGTCTTGACTGAAA | 866 | GGTCAATGGCTAAATCATTCG |
| rs7932189 | 867 | GCAATTCCAGATATCTCTTTAT | 868 | TTATCTACCCATGCTTCTCTC |
| rs7968311 | 869 | GCATAAACAAATGTGTAACGTGGT | 870 | TGTTTTCGTAGTCTTTATTGCT |
| rs8006558 | 871 | TGCTAGCTATATGTAGGTCAGTT | 872 | CGTTAGTTCCCTGGAAAGATCA |
| rs8054353 | 873 | TTGCATAGATGTAGCAGTATTTC | 874 | GACTTTCTTAAAGCTGCACAATCA |
| rs8084326 | 875 | GTTTGCTTGCTTTTACTTTG | 876 | TGTGAAGCACCATTTCTGTTT |
| rs8097843 | 877 | AACAGTGAGGCTCTCCTGTAGC | 878 | CCCATTGTCACCGAGGATA |
| rs9289086 | 879 | CAGAGAGCTCACTTCTAGTTCTGC | 880 | GCTATCTTGGGTCATGAATTTG |
| rs9310863 | 881 | CCTCATGCAATTCAAAGGAA | 882 | CATTTCCCCTAGGTTTGTGC |
| rs9311051 | 883 | GTGGGGCACACAGTGTCTT | 884 | CTTAGATTTGTTCATCTGATGGT |
| rs9356755 | 885 | TTGGGTAGATGCAATGCAAG | 886 | AACCCATATGACTAAGGTGAA |
| rs9544749 | 887 | GCTGAAAATTCACACTGTGGTC | 888 | TGTCATAATGAAGAGCTAGTTGC |
| rs9547452 | 889 | GAGAGGTAAGAGAGAGTATCTTTG | 890 | GAGTTATTTCCCTTAAAAACCAG |
| rs9814549 | 891 | GCTACGCTTGACACCCTTACA | 892 | GGATGCTGTGAGTGCTAAATGA |
| rs9861140 | 893 | GGCACTGCGTCAGCATACTA | 894 | CTGGCTCCTTGCCATCAT |
| rs9919234 | 895 | TAGGCCTCAGAAAGAACGAG | 896 | TGCTAGGCTTACTTCGTTTTC |
| rs9955796 | 897 | AAAATAATTCCCTTTGGTATGC | 898 | CATCATGAATTCTCCCAATGC |
| rs10073918 | 899 | TTGGGTAAATGTGTGACTACGC | 900 | TACCTGGGGCCCTGATTTAT |
| rs10096021 | 901 | GCACTGAAAATGTTAGTGATT | 902 | CCTTAGTGAGGTATTTAGGTTACA |
| rs10197959 | 903 | AGGGAGTTATGATGCCAAGG | 904 | TGATCAGGGGTAGAAGAGATTT |
| rs10233000 | 905 | CGGCTTCCAATCGTATCTTG | 906 | GACAAGTCAGAGAACAAGCTG |
| rs10444584 | 907 | TCATCTGTAACTAATGAACCTTG | 908 | TCAGGAAAGAATGCTACTCA |
| rs10473372 | 909 | AATTGGATGCTGTTTTAACC | 910 | TGCCACATGACAAATTATCACA |
| rs10777309 | 911 | CCAAGGTTTAGCTACATGTATAA | 912 | CTGATAGAAAAATTTCTGTTGTG |
| rs10783507 | 913 | ATTCCTTCCCGCCTTGCT | 914 | ATTCCTGCACAGGCTCAGAC |
| rs10802949 | 915 | AAATGTTCAGTGTAAAAGGCTACA | 916 | AAAGGACTAGCAGCATGTAACTC |
| rs10816273 | 917 | CACTACTTCCCCTTCCCAAA | 918 | AAGATCTGGTAGAAATAAATGGA |
| rs10817141 | 919 | GCTTCCAGGCTAAAAGAAGG | 920 | AAAAAGAAAAGCTGGTTAGG |
| rs10892855 | 921 | CACCTCTATGGTTTAGTCCACTCC | 922 | CCTGGGATTGAAAGCACCTA |
| rs11098234 | 923 | GGAATTGCCACTCTGGAGAA | 924 | AGTGGTCCCCAACAACTTGA |
| rs11119883 | 925 | TCAGATAAAACAATTCCAGTTAC | 926 | ACCCACAGAGGAAAGCCTTG |
| rs11157734 | 927 | CCTGCTGGCACACGTAAGTT | 928 | CCATGGGAATTTGAACCACT |
| rs11166916 | 929 | AACCACAATCCACCTCTTGC | 930 | GCCAAGTCATTAACACAAAGTGA |
| rs11223738 | 931 | CCCACTCTTCTGCTTTACTCCA | 932 | GAGAAGGGGAAAGAGAACAAA |
| rs11247709 | 933 | GGCTTTTTCCACCCAGCTTA | 934 | AGTGGGCAATAATAAACCTT |
| rs11611055 | 935 | GGTGGCTGGAGAAATTGAGA | 936 | AAAGACAATTTGGCTGGTGTTT |
| rs11627579 | 937 | GCTAAGTTGCCTCCAAGCTG | 938 | TTCCCTATTTCTGCCAAAGC |
| rs11636944 | 939 | TTCATGGAGATTTGACCAGTG | 940 | CAGATACTCCTTTTTGGAGAGTCA |
| rs11643312 | 941 | CAGCTAATGCATAAGGGAGATG | 942 | CCAGAACATTTCATCACTCCAA |
| rs11738080 | 943 | GTACAGAGTCCCTGTCTCACA | 944 | CATGATCTGTCTCTCTCACTGAA |
| rs11750742 | 945 | GTGGCAGAACTGACATGCAA | 946 | TGTGGGGGCAGACAGACT |
| rs11774235 | 947 | TCCACCAGAAACCCTTTGG | 948 | CCTCTGTGGAAAGGAAGGAA |
| rs11785511 | 949 | CCCGCTCCAGGTTATTCTC | 950 | AAGAAATCTGAAAAGCAGAGG |
| rs11924422 | 951 | AACTGATTCACATGAGGTTGC | 952 | TTTGAGAGGCAACATTAACAA |
| rs11928037 | 953 | AGTCTGTACAAGGGGCCACA | 954 | TAAGGCTCCTGTGGTAGACG |
| rs11943670 | 955 | CATCATGGAAGGTCCCTCAC | 956 | CAAGATCAAGGCATTGGTAG |
| rs12332664 | 957 | AGGTTCAGATTCTATTTCTGTCA | 958 | CCTTGCCTAAGATAACACAACCA |
| rs12470927 | 959 | TGTTTTGTAATTCCTTTCAGTCA | 960 | CCTCAAATACTGAAGATAGCAAGC |
| rs12603144 | 961 | GACAAGAACTGAAGGCAAAGG | 962 | GGGAGGAACAGAACAACCTTC |
| rs12635131 | 963 | TCGCAGTCTTTTGCATCATT | 964 | TCCAATAGCTACCTTCACCAGAA |
| rs12669654 | 965 | GGTTAAATTCTACTTCGCAACCA | 966 | GCAGTGTAGTCTAACTAGCTGTGT |
| rs12825324 | 967 | CAGCTTCCCAGTTTCTCACA | 968 | AATTGCTACATTCCTGTCTATTG |
| rs12999390 | 969 | GCGGAAAGACATTCCATGTT | 970 | TGCATCTCAATGATATTGCTTTT |
| rs13125675 | 971 | TCTCTGAGAGCAAAGACACT | 972 | TGTGCAATAGTAATAATGGGTCT |
| rs13155942 | 973 | GAGGGTACCTTTCTTTCTCC | 974 | GCTCAGTGTCTGACAAAAGC |
| rs17361576 | 975 | TGGCTGCCTAAAATTATTTACGA | 976 | AAGCAAATAAGGCCATCTAAGAA |
| rs17648494 | 977 | TCAAACAAAAACAGTGTAGGCATT | 978 | GAAAAGTTAAGTCAGAGGCTATCG |

TABLE 6

Excluded SNVs primer pairs

| SNV | SE Q ID NO | First Primer Sequence | SEQ ID NO | Second Primer Sequence | Reasons for exclusion |
|---|---|---|---|---|---|
| rs31036 | 979 | AAGTCACCTAA ATGGCATGA | 980 | AGACACAGCAAGA TGCAAAA | High Unmapped Reads |
| rs42101 | 981 | CAGCAACCCTTT GAAGCAAT | 982 | TGTTTTCTCTTCAA ATGCAA | High Unmapped Reads |
| rs164301 | 983 | TGACTCAGTGGTGAACTGTCT | 984 | GCAGCCCATTAATACTAGCACA | High Unmapped Reads |
| rs232474 | 985 | TGCATTCAAGAGGAAGAAAGG | 986 | TCAGGACGAATTCACAGGAT | Low Depth |
| rs235854 | 987 | ATGAAGGCCAGGCTGTAGG | 988 | GAACATTCACTGCCTTACTCTCA | High Off-Target Reads, Low Depth, High Unmapped Reads |
| rs238925 | 989 | TTCAGTGAAGGGATGGACCT | 990 | GGCCACAGGATCTCCTATCT | High Unmapped Reads |
| rs242656 | 991 | CCAAGTAATCACTTCAACCCTCT | 992 | GCTAGCTACGCCCACGAGAT | High Unmapped Reads |
| rs243992 | 993 | AACTCAAACCTAAGTGCCCC | 994 | GGAATGGAATAGTGTGTGGG | Low Depth, High Unmapped Reads |
| rs251344 | 995 | ACACTGGTCTCAAGCTCCC | 996 | CACACCTGTAATTCTAGCCC | High Off-Target Reads |
| rs254264 | 997 | AGAAGGAAGGATCAGAGAAG | 998 | AGCTTTCCTCCCCACACTG | High Off-Target Reads |
| rs265518 | 999 | TAACAAATTTGCATGTCATC | 1000 | AGAAGCCAGGTGCTGAAGTG | High Off-Target Reads |
| rs290387 | 1001 | GCTGTGTGGAGCCCTATAAA | 1002 | GAATGAAATGGAGTTTGCAG | High Unmapped Reads |
| rs357678 | 1003 | GGCAGTGTTTAAGGTGTTGG | 1004 | AGGTAGTGATTTCTAGGCTTATCA | High Unmapped Reads |
| rs378331 | 1005 | CCTGGAAGTATTCATTCATGTGG | 1006 | GGGACATCTGGGTAGCACTG | High Off-Target Reads |
| rs425002 | 1007 | AAGAGTGTCTCCTCCCTCTG | 1008 | AACTGGAGGCTGTGTTAGAC | High Off-Target Reads |
| rs447247 | 1009 | AAAAACCCCAGGCTCCATTG | 1010 | ATGTCCAGCTGCTTCTTTTC | High Off-Target Reads |
| rs499946 | 1011 | ATGGCTTGTACTTCCTCCTC | 1012 | TTCGGTGGAATAGCAGCAAG | High Unmapped Reads |
| rs516084 | 1013 | AGTATGCCATCATGAAAGCC | 1014 | CTTCTTTGACTAAGGCTGAC | High Unmapped Reads |
| rs602182 | 1015 | GATCTTCCAGGGGGCACT | 1016 | TCATTTTGGTTTCGTTCATT | Low Depth |
| rs621425 | 1017 | CCTTTTGTGGCTTTTCCTCA | 1018 | GGCATTCCAACATGAAAAGG | High Off-Target Reads |
| rs642449 | 1019 | CAGCTGCTGTTCCCTCAGA | 1020 | CCAAAAAACCATGCCCTCTG | High Unmapped Reads |
| rs686106 | 1021 | GGTTCACAGAGCCCAAGTTAC | 1022 | TGAGTCTCTTACTGATCCTGTGAC | High Unmapped Reads |
| rs751834 | 1023 | CTTCCCTCTGCCTCTTTTAGA | 1024 | CCAAAGAGCTCAGGTCTCCA | High Unmapped Reads |
| rs755467 | 1025 | AGGTGAGCATGGGGTTGATA | 1026 | ACCTCTTCCTTCCTCACCAA | High Unmapped Reads |
| rs84227 4 | 1027 | GGCAGCTCCACACACCTTAG | 1028 | TCATCTTTTGGTTTTAGATTGTG | High Off-Target Reads, High Unmapped Reads |
| rs893226 | 1029 | CAACTGCCCGCTTATCCTT | 1030 | AAGACAGCTTGAAGATTCTGG | High Bias |
| rs898212 | 1031 | AAGGTCTAAGGGGGCACAAG | 1032 | ATGGCCACGCTCTTTGTC | High Unmapped Reads |
| rs94977 1 | 103 3 | CCAGATTATCTT CTTCGCCCTA | 1034 | TGATTAGGGTTGGGAAGTGG | High Off-Target Reads |
| rs955105 | 1035 | TTCAGCTCTTCTACTCTGGACTG | 1036 | TGAAACAAGAGAAGACTGGATTTG | High Unmapped Reads |
| rs959964 | 1037 | CAAGTTAGTGAGAAACAGAGTC G | 1038 | GGCCTCTACTCCAAGAAAGC | High Bias |
| rs967252 | 1039 | GTTATATCTCTTTTGTTTCTCTCC | 1040 | TTGGATTGTTAGAGAATAACG | High Bias |
| rs1007433 | 1041 | GTCCAGCTGTGTGATTATCT | 1042 | AGAGGGAGATGGAATAAAAA | Low Depth |
| rs1062004 | 1043 | AAAAATAAACATCCCTGTGG | 1044 | ACATAGCCACCAGCCACACT | High Off-Target Reads, High Unmapped Reads |
| rs1080107 | 1045 | TGCTCTTTTTCTCACAAATGA | 1046 | ATATTGGTCAGTGGGGCAAA | High Off-Target Reads |
| rs1242074 | 1047 | GCACATGAGCTGAGACTGGA | 1048 | TGGCAGTATTACCTGAGCAA | High Off-Target Reads, High Unmapped Reads |
| rs1263548 | 1049 | GCAGCGTCTTGCCTCCTT | 1050 | GCCCAGCTCTTAACACAACA | Low Depth |
| rs1286923 | 1051 | AAAAGGCTGGAGGATGAAGG | 1052 | TCAGAAGGCACCTCTGTCAC | High Off-Target Reads, High Unmapped Reads |
| rs1353618 | 1053 | TGCAACCAAAACTCAGTTATCTA | 1054 | TCCCTTGCCTATCATTGCTT | High Unmapped Reads |
| rs1355414 | 1055 | TTCCCAGCCTTCCAGGAG | 1056 | TACAATGGCTGACTGAGCAC | Low Depth |
| rs1418232 | 1057 | TGATTTAAACCTGATCTTGGTGA | 1058 | ATTCCTGTCCACCCTGGTC | High Off-Target Reads, High Unmapped Reads |
| rs1474408 | 1059 | CCTTTGATCACAAGCAACCA | 1060 | TTACTCTTGGGTCAGGTGCAT | High Unmapped Reads |
| rs1496133 | 1061 | ATGGCAGAAGAGCCCAGAG | 1062 | CGATGCTGACCTTCTGGAGT | High Unmapped Reads |
| rs1500666 | 1063 | GCTGAAAAACCCAGGAATCA | 1064 | GGAGTTGAGGGAGAGGGTCT | High Bias |
| rs1514644 | 1065 | GACAGAATGAAATGCTGTGT | 1066 | CTTTCTAATCCAGCAGCCTCT | High Off-Target Reads, High Unmapped Reads |
| rs1565441 | 1067 | CTGATCCCCGTAAGATCAGC | 1068 | CAGGATGAAACGGTGCAG | High Bias |
| rs1674729 | 1069 | TCTCTGACCTGCTTCCTCGT | 1070 | TAAGGCAATAGGCACCAAGC | High Off-Target Reads |
| rs1858587 | 1071 | AGCAATGGGGTCAGAGTCC | 1072 | AGCTGATTCCTTCCCTGGAT | High Off-Target Reads |
| rs1884508 | 1073 | CCTGATGGAGGATCCACTTG | 1074 | CTGCAAAGCTTCCCATCCT | High Off-Target Reads |
| rs1885968 | 1075 | GGGGATCTTAAAAGCACCAA | 1076 | GACACTCCCACTTCTGCCTA | High Off-Target Reads |
| rs1894642 | 1077 | ATTTCTTCAAGTGTATACAGAGC | 1078 | CAGGCAAACATTCCCTTGTA | High Bias |
| rs1915616 | 1079 | CACTGTTGACTCCAAAACAAAAA | 1080 | CTTCCCACAACAATGAGCTG | High Off-Target Reads, High Unmapped Reads |
| rs1998008 | 1081 | GCAGCTAAGAAAGACTCTCCAA | 1082 | TCTTTGCTCCCCACCTATT | High Unmapped Reads |
| rs2056123 | 1083 | TGAATTCAACTGATGGCACA | 1084 | AAGATTTAATCCTTTGAGATGC | High Unmapped Reads |
| rs2126800 | 1085 | TGAAAGGACCCACCAAATGT | 1086 | TTTTGTTGTGTGTTTGCTTT | Common Deletion in Primer Binding Region |
| rs2215006 | 1087 | TTGCTGGCTTACATTCATTCC | 1088 | TACAGCTCAGCCAGTTCTGC | High Off-Target Reads |
| rs2226114 | 1089 | TGGTTGGTATGGTTATTATTGG | 1090 | GCCTTAGTTTCTCTTTCTGTAAAA | Low Depth |
| rs2241954 | 1091 | GGCCAGCACAAACACACC | 1092 | TCCTAGGACTCTCCCTTTAGA | High Unmapped Reads |
| rs2278441 | 1093 | AATGGGCAGATGAGAGCAAG | 1094 | CCAGTACCTACCCCATGTCC | High Unmapped Reads |
| rs2285545 | 1095 | TCCTTTTGACAGGTCCACATC | 1096 | TGGCCCAATTTTCAGTAACTTC | High Unmapped Reads |
| rs2288344 | 1097 | CACCAGGGGTAGAAGTAAGACG | 1098 | GAGTATCCATGCCCAGAACC | High Bias |
| rs2292467 | 1099 | TGCATGTCTGTATGTGTGTTGG | 1100 | ATGCTCCCACTGCATCCTTA | Low Depth, High Unmapped Reads |
| rs2300669 | 1101 | AAATGAAGAGCCAGCAGCAT | 1102 | CCCACCAACACTAACCTAGCA | High Off-Target Reads |
| rs2300855 | 1103 | ACATCTAGCTGAGGTCAGAA | 1104 | TGTGCAGATTTATGCAAATCAA | High Unmapped Reads |
| rs2362540 | 1105 | GGGAATTTCTCTGGTTGGAG | 1106 | AAACACAGCTTCATGACAAG | High Off-Target Reads, High Unmapped Reads |
| rs2376382 | 1107 | GGACTGAGCATATGTGGAAA | 1108 | CCTGAATTTTTACTTCTTTGCTT | High Unmapped Reads |
| rs2430989 | 1109 | TTGCTGAGTAACAGGAAAACAA | 1110 | TGCTAAACCATTAAATAATCTGG | High Bias |
| rs2442572 | 1111 | GATGCTAAGCCCATCTCCTG | 1112 | AGGGTAGGAAGGATGCAATG | High Unmapped Reads |
| rs2509973 | 1113 | GGAGCGACCACTCTTCATTT | 1114 | CTGAAGGGCTCCCAGGCTA | High Off-Target Reads |
| rs2518112 | 1115 | GAAGATTTTGTAGCTGGTCTTGG | 1116 | CCACAATGGTTTGTAAGATTT | Low Depth |
| rs2545450 | 1117 | TGCGTTCTTTGGAGATAAGACC | 1118 | CACATTTCTCACCCATGTCAA | Common SNPs in Primer Binding Region |
| rs2569456 | 1119 | GTTCCCTCATCTGCCCTTC | 1120 | TGTGAGATGAGTGGAGAGCAA | Low Depth |
| rs2632051 | 1121 | TAAATGTGCCTGGCTTGATG | 1122 | CCCTTTCCTTCCTTGGATGT | High Off-Target Reads, High Unmapped Reads |
| rs2732954 | 1123 | TGCAAGGACACCAGAACAGA | 1124 | CATTTGCACAGCATCTGACC | High Bias |
| rs2786951 | 1125 | GGGTGAGATCAAATTCTTAGGC | 1126 | TTCTAATATGTATTTGGGAGAGAG | High Unmapped Reads |
| rs2822493 | 1127 | GCCATGTTTTCATCTTGTGG | 1128 | TCTGTAAAGGACTTCATGTTTCAT | High Unmapped Reads |
| rs2881380 | 1129 | TCCTGCCATCTTAATAGTCTCACA | 1130 | CTTGTGGCCTCTCATTCTCC | High Unmapped Reads |
| rs2906967 | 1131 | TGTTAATGTAAAATTGCCTCGAT | 1132 | GAGCTCTGGCATTTCTCTGC | High Off-Target Reads |
| rs2920653 | 1133 | TGCTGGAAAGTCATTTTGA | 1134 | TTGGCATTATTTGTGATCC | High Bias |
| rs2993998 | 1135 | CCACACTCCCCAGACCAG | 1136 | GGGAAGACCAGAACTTCAGAAA | Low Depth |
| rs3736590 | 1137 | CTCTTGCCTTCTCATTCACAA | 1138 | CTTTCCTCCCTTTGGGACTC | High Unmapped Reads |
| rs3750880 | 1139 | CCCACGCACTGTACCACA | 1140 | TCAGGGCGAGATACACCTTT | High Unmapped Reads |
| rs3778354 | 1141 | GCCAGCTCAGCTCCTCTCT | 1142 | GAGGGAAATTCGAGCATCAG | High Off-Target Reads, High Unmapped Reads |
| rs3907130 | 1143 | GGCACTCAATAAACATTGACACA | 1144 | GGGAGAGAGGTGTTCTCAGC | High Unmapped Reads |
| rs4075073 | 1145 | CGCAATACCTTCAACAGCAG | 1146 | GGTGGGCTGCATTCATAAAG | High Off-Target Reads |
| rs4313714 | 1147 | TGCCAAGAATCCACTCCAAG | 1148 | GGGGAGGGAGAATTGGACTA | High Unmapped Reads |
| rs4502972 | 1149 | CAAAGAAACAGAATGAAAAAGTGG | 1150 | CACCAACCTGGAATGCTTACT | High Unmapped Reads |
| rs4642852 | 1151 | TGACTGCTCTAAAATCTTTGTCA | 1152 | ATACGCCAAACAGTGAGATG | High Unmapped Reads |
| rs4708055 | 1153 | TGACCTATCTATAACCTGTCCAC | 1154 | TGGGAATTTTAGTTTCTCTGTCT | High Unmapped Reads |
| rs4717565 | 1155 | ATTGATCTATGTGTCTGTAGCTT | 1156 | AATTAAGACAGTGTGGTATTGG | High Off-Target Reads |
| rs4768760 | 1157 | TTCAGAGAGGGACACCCTTG | 1158 | TTCTTCGCAACCACACTTTG | High Bias |
| rs4793426 | 1159 | GAGGCTCTCTGGGGCTTG | 1160 | AGCCTTCCACCTGATTGAAA | High Unmapped Reads |
| rs4845835 | 1161 | AGAGTCATGCATCCTTCATT | 1162 | TGGTGGAGACACAGATCCAA | High Off-Target Reads |
| rs4880544 | 1163 | GCAGCAGGAACCATTCACA | 1164 | CACTTGTGTCCTCCAACATT | High Unmapped Reads |
| rs4903401 | 1165 | CCCCTCAGAGTGATGACTGG | 1166 | CTCCTGACCCAGCCACTTT | High Unmapped Reads |
| rs4909472 | 1167 | GAAAATCTTGTGGAGCCTGAA | 1168 | AGAGAGGAGATGGGGGAAAG | High Unmapped Reads |
| rs4909666 | 1169 | TGAGCCTACACTAACACATCA | 1170 | GCCCTAATGTAAACTAAAGACGTT | Low Depth |
| rs4927069 | 1171 | GGAAATGTGACCCTCACAGG | 1172 | TTTTCCATACCTAAAGAACG | Low Depth, High Unmapped Reads |
| rs4945026 | 1173 | CATCATCTCTTCCTTATGTTCTCC | 1174 | GGCCTGGGGGTGCTAATG | High Off-Target Reads |
| rs5009912 | 1175 | GGGTGGTCTGGTGATGTGTT | 1176 | GCTATGCCAAGGGAACCTAGA | Low Depth |
| rs6082979 | 1177 | GGGAGTACTCTCCAAAGC | 1178 | CCTCCTGTCACTTTCCCTCA | High Off-Target Reads, High Unmapped Reads |
| rs6088301 | 1179 | TGCTCCACAGATGACACAGT | 1180 | TGGAATGTGATGGATGAGA | High Bias |
| rs6124059 | 1181 | AGCCCTGCTTCAGCTTCTG | 1182 | TTGACTACTGGAACTTGGAGAGG | High Unmapped Reads |
| rs6134639 | 1183 | TGGAAACTTCTTGTGGACCT | 1184 | GTGGGTGGAAGACTTGCTCT | High Unmapped Reads |
| rs6499618 | 1185 | TTTCTGGGCCACCTACAAGT | 1186 | CCCAAGGTTCTGGGCTAAG | High Off-Target Reads |
| rs6538276 | 1187 | CCTCCTCCTCACACTGCTTC | 1188 | CCCTTTCTTAGCTCCTGACCA | High Off-Target Reads, High Unmapped Reads |
| rs6560430 | 1189 | GGTCTAAAGGGAGAGTAGGAGGTC | 1190 | GAATGGTCTTTTCGTCATTCC | High Unmapped Reads |
| rs6602240 | 1191 | CTTTCCCAAAACCCCACACT | 1192 | CACACACAAGGAAAAACAGGA | High Unmapped Reads |
| rs6681073 | 1193 | GCTGGATGGAGGGTGAGG | 1194 | TGCCTGCCTGTTAGAACATC | Low Depth |
| rs6682943 | 1195 | GGCAATCCGAAGTCTAAGAGA | 1196 | TGGAACCAACAACCTATCATCA | High Bias |
| rs6700298 | 1197 | GACTGGTACTTCCCCAAGGA | 1198 | TGAAAATCCATTTGGTAGTTGCT | High Unmapped Reads |
| rs6714809 | 1199 | AAAATGACTGTCCCCTATCT | 1200 | TGGTAAGTGGGATGATACTGAGC | High Unmapped Reads |
| rs6728087 | 1201 | AAGCATAGAAGGAAAAACAGATTG | 1202 | CCCCTGAATGAAACTATTGAGC | High Bias |
| rs6765108 | 1203 | AGCAAGGGAGGGAAGACACC | 1204 | TTGTCAATCCTTGCTCTACCC | High Off-Target Reads, High Unmapped Reads |
| rs6788750 | 1205 | TGAAGGGTAGATATGAAGTTTTTC | 1206 | TAATCTTTGGACTCCTTGAA | High Bias |
| rs6863383 | 1207 | TGATCCCATGTATTTAAACCT | 1208 | CCCCTGAAATGAGAGTCACC | High Bias |
| rs6893628 | 1209 | CAAAATAAACCCAGGCAAAAA | 1210 | CTTTAACAAATATAGGGCGATTT | High Bias |
| rs6986644 | 1211 | AAGTACCAAAAAGGCACATCG | 1212 | TCCCCCTAAGATCAGGAACA | High Unmapped Reads |
| rs6994806 | 1213 | TGGAACAGCAACTTGCAAAC | 1214 | AAGAGTGTAAATGGGTCCTGA | High Unmapped Reads |
| rs7098657 | 1215 | CTCCCCTGAACCTGAGTGAC | 1216 | TGCTCACATTTCATTGACCAG | High Off-Target Reads |
| rs7133402 | 1217 | TGAGGTGGGAAGAAACACAA | 1218 | TGCGACTGGATACTATTTTTGG | High Unmapped Reads |
| rs7157032 | 1219 | AGTTGCATGGAGTGGCTGA | 1220 | TGTTGGTGCATTCAGAGAGC | High Unmapped Reads |
| rs7195624 | 1221 | CAAGTAATTCTTACCAGCCTTT | 1222 | AGGCTACAAAAAGGCAGCAG | High Unmapped Reads |
| rs7251148 | 1223 | AAGGAAACGGCCCCAGAG | 1224 | GACCCTGTGGACTGAGAACC | High Unmapped Reads |
| rs7479857 | 1225 | TCAGAGCACTCTGCATTCCA | 1226 | CTTTTTAAAGCCAGAAAAATGG | High Unmapped Reads |
| rs7521976 | 1227 | AGAATCATATGACACATGGAA | 1228 | CAGCTTATCTTTATCTGTTTGCTT | High Unmapped Reads |
| rs7564063 | 1229 | CACTTTGCAGCCAATCCATA | 1230 | CAGATCTGATTTCCTGGAG | High Bias |
| rs7608890 | 1231 | TCCATACAGGAAGATCCATTAAGA | 1232 | GTGCAGTTTGGGCTACAAGA | Low Depth |
| rs7684457 | 1233 | TGCTGCCAGAAGCAACCTAC | 1234 | AGAAAGTTGTGCCAAGTGCT | High Off-Target Reads, High Unmapped Reads |
| rs7745188 | 1235 | TGTCTGGAAATCATTGCTTCA | 1236 | CATAAAGCTAAAAGATTGGACA | High Off-Target Reads |
| rs7763061 | 1237 | CAAATCAGTGTGCCCCAAC | 1238 | GTTTTGCCCAGAGGTCATGT | High Unmapped Reads |
| rs7820286 | 1239 | GCTCTTCCCTCAGTGGCTTA | 1240 | CTATCATTTCTCCCCAACACA | High Unmapped Reads |
| rs7830700 | 1241 | CTGGATTTCAAATTGTTTCA | 1242 | TCAAGTATCTAGTTGTGATAGCC | High Bias |
| rs7833328 | 1243 | TAGAGCAGCTAGGGGACTGC | 1244 | CGAGACTGTTCACCCTTTGG | High Off-Target Reads, High Unmapped Reads |
| rs7982170 | 1245 | ATGCCAGACTTCACCACTGC | 1246 | TTTCAGTTTTGTTATGTGGCTA | High Off-Target Reads |
| rs8053194 | 1247 | TTGAAGTTAGTTCTTTGTGGATGG | 1248 | ATCAACTCCCCACCTGGAAG | High Unmapped Reads |
| rs9300647 | 1249 | TTTTCCCTCATTAGCTGCATT | 1250 | TGATTCCAGTTCACAGTAGTCCA | High Unmapped Reads |
| rs9371705 | 1251 | CATTTCCAGCTGACTGGTTA | 1252 | ACCCTGAGGAGGGGCTAGT | High Bias |
| rs9377381 | 1253 | GCCCAGTAGCACTGCTCTTC | 1254 | AGATCACCAAGGCAGAAACC | High Off-Target Reads |
| rs9405991 | 1255 | CCGAGAACGCTCTGAGTTG | 1256 | GGCAGCAACAGGAAATAGCA | High Bias |
| rs9522306 | 1257 | ACAGGAGTGGCTCGGTCA | 1258 | CACTGCAGGAAATGCAGCTT | High Unmapped Reads |
| rs9864296 | 1259 | CGAAATCCATAGGACCTACA | 1260 | AGCTACACTATTTCCATGTGAC | High Unmapped Reads |
| rs9881075 | 1261 | AACAAGAAAGGCAGGGAAGG | 1262 | CTGGGTCACGCCTCTTGA | High Unmapped Reads |
| rs10041720 | 1263 | TACAAACAGTGGGGCAACAA | 1264 | GCCAGGCATGGGCTTAAT | High Bias |
| rs10106215 | 1265 | TTCGTCTTTCAGCAATTTGA | 1266 | AACAGAAAGAGAGTTACATCTACA | High Bias |
| rs10142058 | 1267 | CCTCATGACCTAACCACCTC | 1268 | CCCCCAATGCAAGAGTGTT | High Off-Target Reads |
| rs10444986 | 1269 | TTTCACAGTGGAATGAATCG | 1270 | GCCCAGGACACACAAAAA | High Unmapped Reads |
| rs10765992 | 1271 | CTGGTCCTCTGTGAATTGAA | 1272 | CACCGAATCTATATCTGTGAGG | Low Depth, High Unmapped Reads |
| rs10787889 | 1273 | TCTTTATGTGGCCTTCACTTG | 1274 | TATGCTGAAGCTGCCATCCT | High Off-Target Reads |
| rs10790395 | 1275 | GGGCAGGAAACAGGGACTA | 1276 | GCTGTCCTATTTCAGGTTGCAT | High Unmapped Reads |
| rs10800542 | 1277 | TCCACTGGAATTGGTAGACAGA | 1278 | AGCAATCATCCTAGGAGGTCA | High Unmapped Reads |
| rs10815682 | 1279 | TTCTGACTTCACAGAGGGTA | 1280 | GGGCAAGTCACTTAGCATTT | High Unmapped Reads |
| rs10874506 | 1281 | TTCTCAGACTTCAAAGCAAAGG | 1282 | TGAAAAGATACCTAAAATCAAGG | High Unmapped Reads |
| rs10906984 | 1283 | GAGAAGAACCAGACAGAACACG | 1284 | ATTTCTGCAGCCCTGTGACT | High Unmapped Reads |
| rs10952780 | 1285 | CATGAAAAATAAGGAAATGCTGA | 1286 | TCCTAAGTTTTTCTGATCTGTGG | High Unmapped Reads |
| rs11058137 | 1287 | GCCTCAGTTTCCTCCTCAGA | 1288 | CCTCTCAACAACCCAGGTACT | High Bias |
| rs11153132 | 1289 | ACTGTGGCTCCAGCATGAA | 1290 | AGTCCAGGCACCACTGCTAC | High Off-Target Reads |
| rs11216096 | 1291 | GCTGGAAGGAGAGAAACACG | 1292 | ATGGCCACTAGAGGGGAGTC | High Off-Target Reads, High Unmapped Reads |
| rs11705789 | 1293 | GCATCCTGTGGTGGGAAG | 1294 | TGGTCAATAAGCCTGTTCCA | High Bias |
| rs11714718 | 1295 | GGTCAGGACCTGTTTTCTCAA | 1296 | TCAATAACTGCTGGAGATGTGG | High Off-Target Reads, High Unmapped Reads |
| rs11745637 | 1297 | GCCCAATCTAATCATGTGAGG | 1298 | GCAGCCAAGAAAGGCTGT | Low Depth, High Unmapped Reads |
| rs11786747 | 1299 | GGAAAGCAGTGAAGACAGCA | 1300 | TCCTCTTCCCCAGAACTTGA | High Unmapped Reads |
| rs12210929 | 1301 | GTTGGGGCAGTACTCAGCAG | 1302 | TCCTTTACTACATCATGGGTCA | Low Depth |
| rs12287505 | 1303 | GGCCTCCCCTTCATTCAA | 1304 | TTGAACTAGTTTATACACCCAGAA | High Off-Target Reads |
| rs12321981 | 1305 | CACACATACACAAAATAAAGGT | 1306 | CAAAGAAGAAGGAGCAAGG | High Unmapped Reads |
| rs12349140 | 1307 | TTATCCAGGACAGGAAGCTG | 1308 | CCCGGTGATAACAGAACGAT | High Off-Target Reads, High Unmapped Reads |
| rs12448708 | 1309 | CATGGGACTCTAGAGGTAGAA | 1310 | TTTTAATCTCTCTTGCTCTCC | Low Depth, High Unmapped Reads |
| rs12500918 | 1311 | TCATAGAGTAAGCCAGATATAAGC | 1312 | TTTACCAGCCAGCTCAGTCC | High Off-Target Reads, High Unmapped Reads |
| rs12554667 | 1313 | TCCTGAAGGGTAAGCAGGAA | 1314 | ACCAAGGTCTTCCCTCTGC | High Off-Target Reads, Low Depth |
| rs12660563 | 1315 | AGGTCAGCTCAGGGTGAAGT | 1316 | GCTCCATTGAAGGGTAAAGG | High Off-Target Reads |
| rs12711664 | 1317 | TGGAATAGAATGCAATCCTGA | 1318 | AGCCCACACAGGTTGGTAAG | High Unmapped Reads |
| rs12881798 | 1319 | CAGATGCTGCAGGAAACAGA | 1320 | GTGGATCACAGGGTCACCTC | High Off-Target Reads, High Unmapped Reads |
| rs12917529 | 1321 | CCTCAAGCTGGCCTGCAA | 1322 | AAGGCAGGCAAGACGTAGC | Low Depth, High Unmapped Reads |
| rs13019275 | 1323 | CAAATATACTGATTCTGTGGCAAA | 1324 | TGATGCATTGAGATTTTGATGA | High Unmapped Reads |
| rs13042906 | 1325 | CGTCTCCCACATTCTTTTGG | 1326 | GGTAGGCTTTGTAACTTGCACTG | High Bias |
| rs13267077 | 1327 | TGAATCCTGGCTGGGAAA | 1328 | GCCTCACCTACAAAGCTTATTCA | High Unmapped Reads |
| rs13362486 | 1329 | TGCAGTTTGCTATGCAGTCTTT | 1330 | TGAAGCTACACAGATAAGAAGC | High Unmapped Reads |
| rs17077156 | 1331 | TCATTCTGGGTTACCCTTTTG | 1332 | GCCAGGAAAAGACAGTGCAT | High Unmapped Reads |
| rs17382358 | 1333 | TCTCAGCACAGAGAAGGTGCT | 1334 | GCACATTTATTCACTCAGCAAA | Low Depth |
| rs17699274 | 1335 | TGTCCTCTGTAAACCAGACAA | 1336 | CATTTTCCAAGGTTGTTTCTGT | High Unmapped Reads |

EXAMPLE 3 Validation of SNV panel multiplex PCR on control paternity testing samples Genomic DNA previously used in College of American Pathologists (CAP) proficiency testing at the DNA Identification Division was used to simulate cfDNA neonatal and prenatal paternity testing. CAP proficiency cases encompass genomic DNA from a mother, child, confirmed father, and excluded father. Three proficiency testing cases were analyzed at varying simulated fetal fractions.

Genomic DNA concentration of all individuals was measured using a double stranded DNA specific fluorescence assay on a Promega Quantus device. To simulate a mixed profile of fetal/maternal cfDNA, genomic DNA from the child was mixed with the maternal genomic DNA at various proportions so that the fetal fractions in the mixtures were at 2%, 10% and 20%, respectively. These mixtures simulate the expected range of fetal fractions. Mixtures were then diluted to a concentration equal to 800 genome equivalents (gEqs) followed by SNV amplification using primers listed in Table 5. Isolated genomic DNA from individuals in family studies (mothers, children, and potential fathers) were genotyped in individual reactions using the same SNV panel amplification. In prenatal cfDNA paternity testing, a single-source fetal genomic DNA will not be available, but it was analyzed separately here for verification of fetal associated mixture SNVs. Duplicates of de-identified clinical maternal cfDNA were also assayed in parallel to synthetic mixtures. Although no maternal or paternal genomic material was available for analysis, the number of extracted fetal SNVs could be compared to synthetic mixtures and the feasibility of paternity testing could be evaluated.

After SNV amplification and Illumina sequencing on a HiSeq2500, reads were aligned to the human genome and counted for each possible nucleotide at the SNV location. The number of reads for each nucleotide at a given SNV was then converted into the reference allele frequency (RAF) by the formula: reference allele frequency = number of reads for reference allele/ (number of reads for reference allele + number of reads for alternative allele). For the pure maternal, child, and potential paternal genomic DNAs, the RAF was used to determine if the individual was homozygous reference allele, homozygous alternate allele, or heterozygous. Determination is based on a conservative RAF cutoff of 0-0.1 RAF, indicating homozygous alternate allele, 0.9-1 RAF indicating homozygous reference allele, and 0.4-0.6 RAF indicating heterozygous. After determining genotypes, they were uploaded into Familias3 open-source software for relationship confirmation. The standard for paternity testing of trios, i.e., mother, child, and alleged father, requires a likelihood ratio (LR) over 10,000. When analyzed as unmixed DNAs, the correct father was identified in all three proficiency testing cases with an LR over 1,000,000,000, and the incorrect father was excluded in all three cases with an LR of 0 and multiple exclusion SNVs (data not shown).

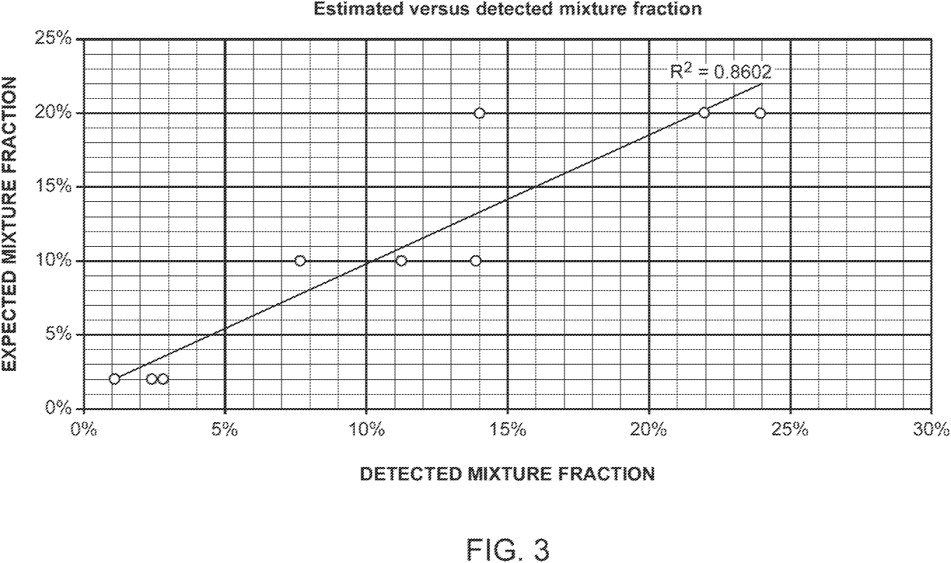

Similar to the above, the reference SNV allele frequencies were determined for the synthetic mixture model samples and the clinical cfDNA samples. After allele frequency calculation, k-means clustering analysis was performed on synthetic mixtures and cfDNA samples to extract the population of SNVs (informative SNVs) where the child genotype could be determined. Percent of modeled fetal DNA and fetal cfDNA fractions can be calculated using the average allele frequencies of informative SNVs. To analyze whether the targeted fetal fraction of the synthetic mixtures was successful, the estimated versus detected fetal fraction for the proficiency testing synthetic mixtures was plotted (FIG. 3). There was a positive correlation between the estimated and the detected fetal fraction (p=0.003, R2=0.86), indicating that the method simulating cfDNA mixtures was successful, and of the use of these SNVs can accurately determine fetal fraction. Accurate detection of fetal fraction confirms that the selected informative SNVs are associated with the fetal-specific DNA. Fetal fraction can also serve as a quality control metric - if the fetal fraction is sufficiently high, the paternity index may be inaccurate and cause mis-classify paternity.

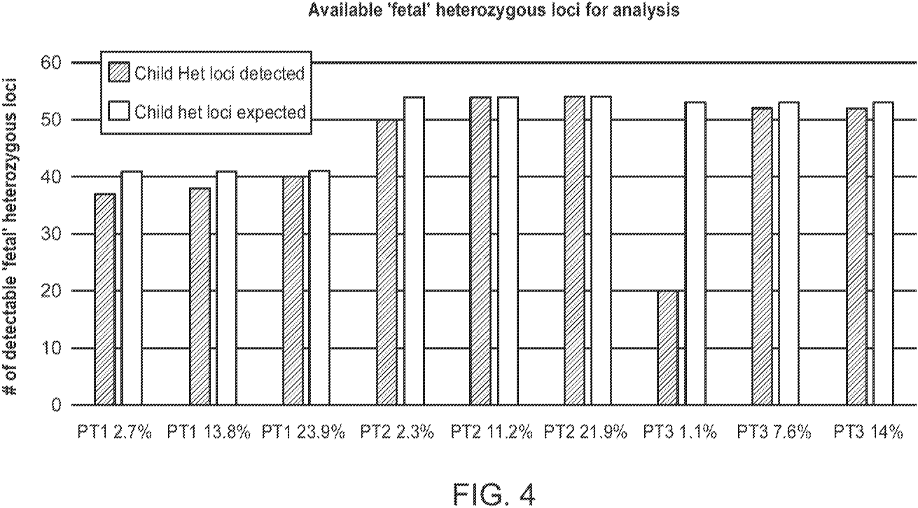

The methods were then performed on three proficiency test mixtures, each proficiency mixture was produced by mixing the genomic DNA from a mother and her child at low concentrations to simulated cfDNA in samples obtained from pregnant mothers. PT1, PT2, and PT3 are from three different mothers. For example, PT3 14% refers to a mixture containing mother#3 and her child’s genomic DNA are mixed in a manner such that the child’s genomic DNA accounts for 14% of total genomic DNA in the mixture. Proficiency test 3 (PT3) was lower than expected for all three mixtures, while proficiency test 2 (PT2) and proficiency test 1 (PT1) were slightly elevated. The detected fetal fraction in further analysis will be indicated by and is based on the SNV measured mixture percent (e.g., PT3 14% = PT3 mixture at 14% fetal fraction). See FIG. 4.

Even one or two base miscalls during fetal fraction genotyping can lead to false paternal exclusions during paternity index (aka “likelihood ratio” or “LR”) calculations. Therefore, further analyses on k-mer inferred fetal genotypes were conducted to ensure no false genotypes were called. Specifically, after defining the maternal genotype from maternal genomic DNA only genotyping, only loci where the mother was homozygous at a location were taken into consideration. For these loci the following steps were taken. All cfDNA reads not above the mother’s genotyping frequency by 0.005 were removed. All loci below 400 total reads were removed. The remaining pool of loci indicated where the mother was homozygous and child was heterozygous at a given SNV. Each proficiency test mixture was assayed for the total number of child heterozygous/maternal homozygous loci, which is compared to the potential number of child heterozygous genotypes that were determined by child genomic DNA genotyping (FIG. 4). The results showed that all mixtures but PT3 1.1% returned over 90% of potential loci, and ranged from 37 to 52 fetal genotypes for paternity calculations. PT3 1.1% only returned 37% of loci, most likely due to low fetal fraction input. Most importantly, no false fetal genotype calls were made.

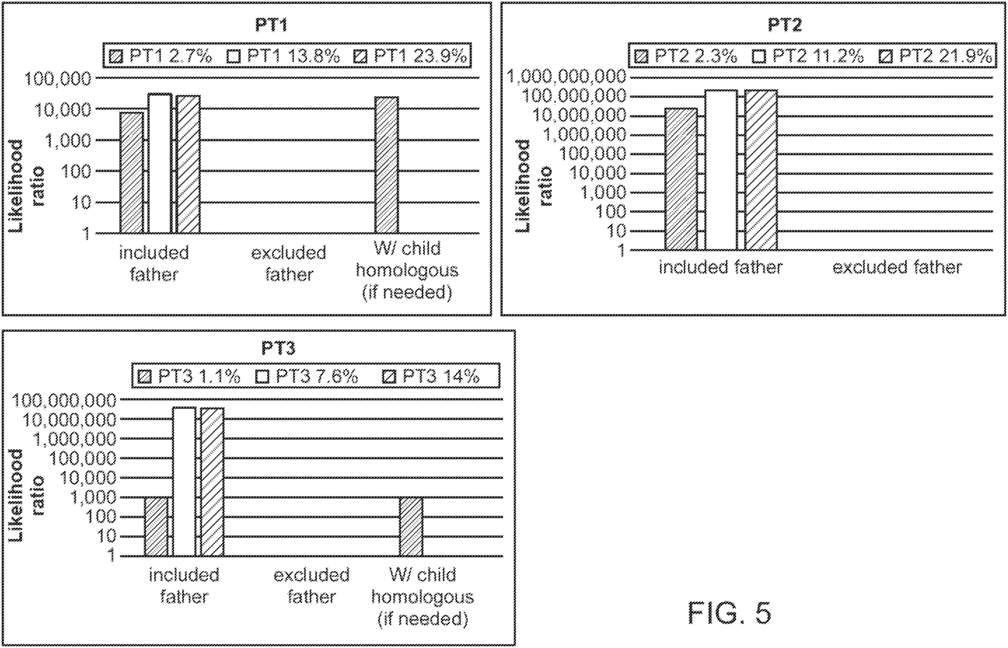

Extracted fetal heterozygous, maternal, included paternal, and excluded paternal genotypes were input into Familias3 for LR calculations. For all nine mixtures, the LR of the excluded father was 0. Seven mixtures were able to reach internal LR thresholds (>10,000) using fetal heterozygous loci alone (FIG. 5). Two mixtures (each about 2%) did not reach statistical significance but did not exclude the biological father. In instances where 1) the mother was homozygous and the child was heterozygous, 2) the LR was inconclusive, and 3) the alleged father was not excluded, further analyses were performed. Specifically, loci where the mother was heterozygous and child homozygous were analyzed. To ensure no false fetal homozygous genotypes were analyzed, a minimum and maximum heterozygous range were set at each locus based on all genomic genotypes of the sequencing run. Any potential fetal fractions in this range were removed. The percent fetal fraction was then added to or subtracted from the maternal heterozygous allele frequency and all potential loci below or above this range were removed. Remaining loci were considered to be child homozygous and used in LR calculations. For PT1 2.7%, multiple fetal genotypes were able to be extracted raising the LR to above 10,000 (FIG. 5). However, no further fetal genotypes could be determined for PT3 1.1%. Therefore, the limit of detection for this assay is estimated to be 2-4%.

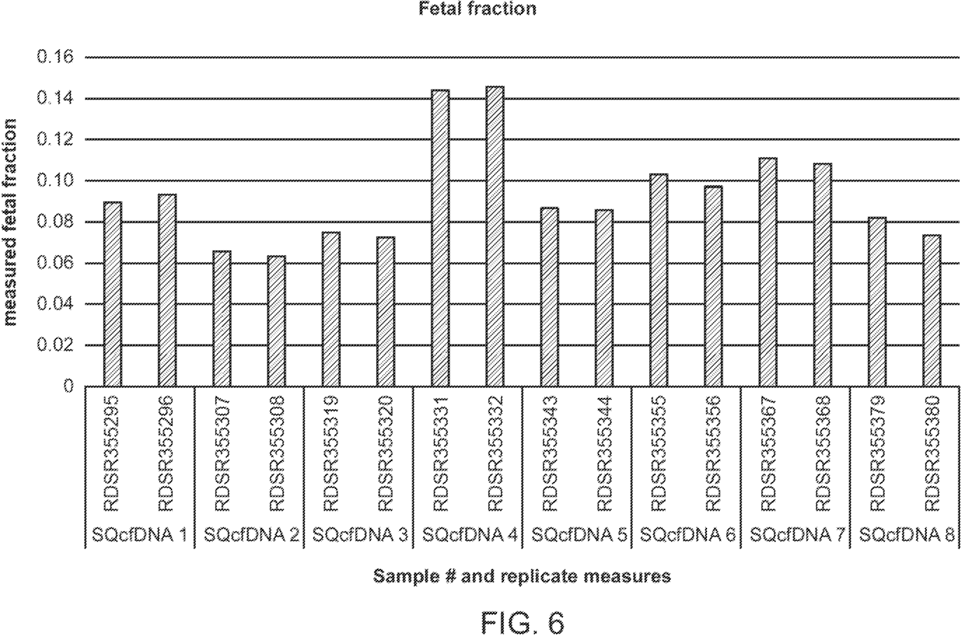

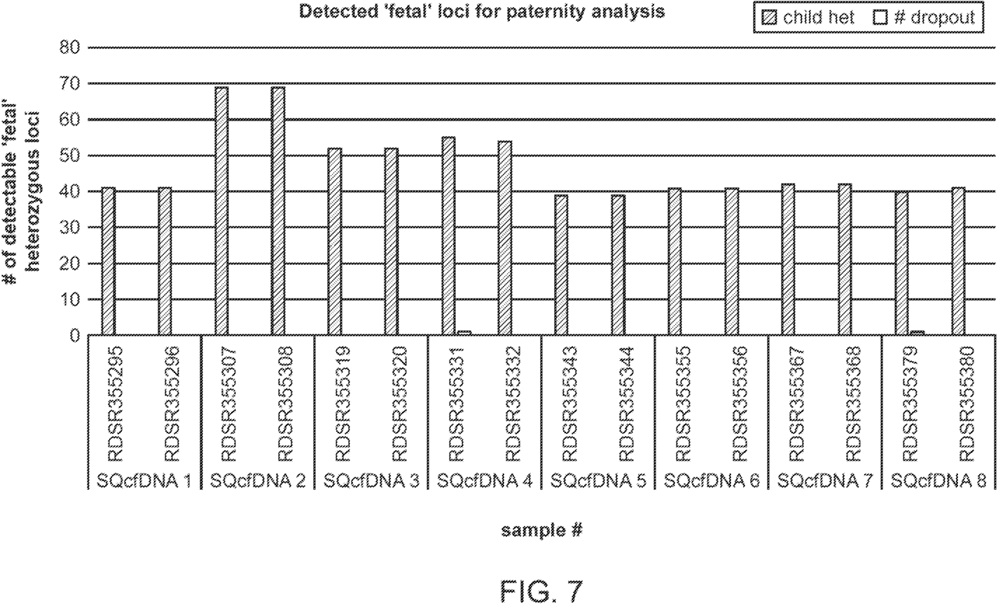

The bioinformatics analysis that was used to analyze proficiency testing samples was also used to analyze the percent fetal fraction for the de-identified clinical maternal cfDNA sample duplicates. The fetal fraction for samples ranged from 6.3% to 15.5%, well above the projected limit of detection of 2-4% (FIG. 6). Although the maternal genomic DNA was not available for genotyping, fetal specific heterozygous genotypes were extracted for comparison between sample duplicates to determine if loci number would be able to establish statistical significance if further paternity testing was performed (FIG. 7). The number of fetal genotypes extracted, 39-69, is projected to return a conclusive paternity testing result. When comparing the duplicate samples, only two displayed any discrepancies. Further investigation revealed this was most likely due to low read counts with the missing loci just below the threshold, and not a false inclusion of a fetal allele.

### INCORPORATION BY REFERENCE

Each and every publication and patent document referred to in this disclosure is incorporated herein by reference in its entirety for all purposes to the same extent as if each such publication or document was specifically and individually indicated to be incorporated herein by reference.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed and their equivalents.

1. A method of determining paternity of a fetus in a pregnant mother comprising:
(a) obtaining genotypes for one or more polymorphic nucleic acid targets in a genomic DNA sample obtained from an alleged father,
(b) isolating cell-free nucleic acids from a biological sample obtained from the pregnant mother comprising fetal nucleic acids;
(c) measuring the frequency of each allele of one or more polymorphic nucleic acid targets in cell-free nucleic acids;
(d) select informative polymorphic nucleic acid targets from the one or more polymorphic nucleic acid targets,
(e) determining the measured allele frequency of each allele of the selected informative polymorphic nucleic acid targets and thereby determining fetal genotypes based on the measured allele frequency for each selected informative polymorphic nucleic acid targets, and
(f) determining paternity status of the fetus based on the genotypes of the mother, alleged father and the fetus for the informative nucleic acid targets.

2. The method of claim 1, wherein step (a) further comprises obtaining genotypes for the one or more polymorphic nucleic acid targets in a genomic DNA sample obtained from the pregnant mother.

3. The method of claim 1, wherein step (e) further comprises by comparing the measured allele frequency to a threshold of respective polymorphic nucleic acid targets.

4. The method of claim 1, wherein step (f) comprises determining paternity index for each informative polymorphic nucleic acid targets, determining a combined paternity index for all informative polymorphic nucleic acid targets, which is the product of the paternity indexes for each informative polymorphic nucleic acid targets.

5. The method of claim 4, wherein the paternity index is determined by inputting the genotypes of the mother and alleged father and fetal genotypes for each of the informative polymorphic nucleic acid targets into a paternity determination software.

6. The method of claim 4, wherein the alleged father is determined to be a biological father if the combined paternity index is greater than a predetermined threshold.

7. The method of claim 1, wherein step (c) comprises determining measured allele frequency based on the amount of each allele of one or more polymorphic nucleic acid targets in cell-free nucleic acids.

8. The method of claim 1, wherein the informative polymorphic nucleic acid targets are selected by performing a computer algorithm on a data set consisting of measurements of the one or more polymorphic nucleic acid targets to form a first cluster and a second cluster,
wherein the first cluster comprises polymorphic nucleic acid targets that are present in the mother and the fetus in a genotype combination of AAₘₒₜₕₑᵣ/AB_{fetus}, or BBₘₒₜₕₑᵣ/AB_{fetus}, and/or
wherein the second cluster comprises SNPs that are present in the mother and the fetus in a genotype combination of ABₘₒₜₕₑᵣ/BB_{fetus} or ABₘₒₜₕₑᵣ/AA_{fetus}.

9. The method of claim 1, wherein said polymorphic nucleic acid targets comprises (i) one or more SNVs, (ii) one or more restriction fragment length polymorphisms (RFLPs), (iii) one or more short tandem repeats (STRs), (iv) one or more variable number of tandem repeats (VNTRs), (v) one or more copy number variants, (vi) insertion/deletion variants, or (vii) a combination of any of (i)-(vi).

10. The method of claim 1, wherein said polymorphic nucleic acid targets comprise one or more SNVs.

11. The method of claim 10, wherein the one or more SNVs exclude any SNV, the reference allele and alternate allele combination of which is selected from the group consisting of A_G, G_A, C_T, and T_C.

12. The method of claim 1, wherein each polymorphic nucleic acid target has a minor population allele frequency of 15%-49%.

13. The method of claim 1,wherein the SNVs comprise at least two, three, or four or more SNVs of SEQ ID NOs: in Table 1 or Table 5.

14. The method of claim 1,wherein the biological sample in step (b) for is one or more of blood, serum, and plasma.

15. The method of claim 1, wherein identifying one or more cell-free nucleic acids as fetus-specific nucleic acids comprising applying a dynamic clustering algorithm to
(i) stratify the one or more polymorphic nucleic acid targets in the cell-free nucleic acids into mother homozygous group and fetus heterozygous group based on the measured allele frequency for a reference allele or an alternate allele of each of the polymorphic nucleic acid targets;
(ii) further stratify recipient homozygous groups into non-informative and informative groups; and
(iii) measure the amounts of one or more polymorphic nucleic acid targets in the informative groups.

16. The method of claim 1, wherein fetal-specific nucleic acids are detected if the deviation between the measured frequency of a reference allele of the one or more polymorphic nucleic acid targets and the expected frequency of the reference allele in a reference population is greater than a fixed cutoff,
wherein the expected frequency for the reference allele is in the range of 0.00-0.03 if the mother is homozygous for the alternate allele, 0.40-0.60 if the mother is heterozygous for the alternate allele, or 0.97-1.00 if the mother is homozygous for the reference allele.

17. The method of claim 16, wherein the mother is homozygous for the reference allele, and the fixed cutoff algorithm detects fetus-specific nucleic acids if the measured allele frequency of the reference allele of the one or more polymorphic nucleic acid targets is less than the fixed cutoff.

18. The method of claim 16, wherein the mother is homozygous for the alternate allele, and the fixed cutoff algorithm detects fetus-specific nucleic acids if the measured allele frequency of the reference allele of the one or more polymorphic nucleic acid targets is greater than the fixed cutoff.

19. The method of claim 16, wherein the fixed cutoff is based on the measured homozygous allele frequency of the reference or alternate allele of the one or more polymorphic nucleic acid targets in a reference population.

20. The method of claim 16, wherein the fixed cutoff is based on a percentile value of the measured distribution of the measured homozygous allele frequency of the reference or alternate allele of the one or more polymorphic nucleic acid targets in a reference sample set.

21. The method of claim 14, wherein the individual polymorphic nucleic acid target threshold algorithm identifies the one or more nucleic acids as fetus-specific nucleic acids if the measured allele frequency of each of the one or more of the polymorphic nucleic acid targets is greater than a threshold.

22. The method of claim 21, wherein the threshold is based on the measured homozygous allele frequency of each of the one or more polymorphic nucleic acid targets in a reference sample set.

23. The method of claim 21, wherein the threshold is a percentile value of a distribution of the measured homozygous allele frequency of each of the one or more polymorphic nucleic acid targets in the reference sample set.

24. The method of claim 1, wherein the amount of one or more polymorphic nucleic acid targets is determined in at least one assay selected from high-throughput sequencing, capillary electrophoresis, or digital polymerase chain reaction (dPCR).

25. The method of claim 24, wherein detecting the frequency of each allele of the one or more polymorphic nucleic acid targets comprises targeted amplification using a forward and a reverse primer designed specifically for the allele or targeted hybridization using a probe sequence that comprises the sequence of the allele and high throughput sequencing.

26. The method of claim 24, wherein the one or more polymorphic nucleic acid targets comprise an SNV, and wherein detecting the amount of an allele of the SNV comprises hybridizing at least two probes to the polymorphic nucleic acid target comprising the SNV, wherein the two probes are ligated to form a linked probe when one of which comprise a nucleotide that is complementary to the allele of the SNV.

27. The method of claim 26, wherein the detecting the amount of the allele further comprises hybridizing primers annealed to the linked probe to produce amplified linked probe and sequencing the amplified linke probe.

28. A system for determining paternity comprising one or more processors; and memory coupled to one or more processors, the memory encoded with a set of instructions configured to perform a process comprising:
obtaining genotypes for one or more polymorphic nucleic acid targets in a genomic DNA sample obtained from an alleged father,
determining the amount of each allele of one or more polymorphic nucleic acid targets in cell-free nucleic acids from a sample obtained from a pregnant mother,
select informative polymorphic nucleic acid targets from the one or more polymorphic nucleic acid targets,
determining the measured allele frequency of each allele of the selected informative polymorphic nucleic acid targets and thereby determining fetal genotypes based on the allele frequency for each selected informative polymorphic nucleic acid targets, and
determining the paternity status of the fetus based on the genotypes of the mother, alleged father and the fetus for the informative nucleic acid targets.

29. A non-transitory machine readable storage medium comprising program instructions that when executed by one or more processors cause the one or more processors to perform a method of determining paternity status of claim 1.
